(19) United States
(12) Patent Application Publication
(10) Pub. No.: US 2023/0304010 A1
(43) Pub. Date: Sep. 28, 2023

(54) COMPOSITIONS AND METHODS FOR TREATING VIRAL INFECTIONS

(71) Applicant: AADIGEN, LLC, Pacific Palisades, CA (US)

(72) Inventors: Neil P. DESAI, Pacific Palisades, CA (US); Gilles DIVITA, St. André De Sangonis (FR)

(21) Appl. No.: 18/017,644

(22) PCT Filed: Jul. 23, 2021
(86) PCT No.: PCT/US2021/043077

§ 371 Date: Jan. 23, 2023

(30) Foreign Application Priority Data
Jul. 24, 2020 (FR) FR2007849

(51) Int. Cl. C12N 15/113; A61P 31/14

(52) U.S. Cl. CPC C12N 15/1131; A61P 31/14; C12N 2310/14; C12N 2320/32

(57) ABSTRACT

The present application provides peptides and nucleic acids that are useful for treating virus (e.g., SARS-CoV-2) infection. Exemplary peptides comprise chimeric peptides and blocking peptides that block the interaction between SPIKE and ACE2. Exemplary nucleic acids include siRNAs that specifically target SARS-CoV-2. The present applications also provide complexes and nanoparticles further comprising a second peptide (e.g., a cell-penetrating peptide) that promotes intracellular delivery of the peptides and nucleic acids.

### CROSS-REFERENCE TO RELATED APPLICATION

This application claims priority to French application No. FR2007849, filed on Jul. 24, 2020, the contents of which are incorporated by reference in their entirety for all purposes.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 737372001341SEQLIST.TXT, date recorded: Jul. 23, 2021, size: 59 KB).

### FIELD OF THE APPLICATION

The present application relates to inhibitory peptides, nucleic acids (e.g., siRNAs), complexes, nanoparticles, and compositions for treating virus (e.g., SARS-CoV-2) infection.

### BACKGROUND OF THE APPLICATION

The emergence of the novel human coronavirus SARS-CoV-2 in Wuhan, China has caused a worldwide epidemic of respiratory disease (COVID-19). Vaccines and targeted therapeutics for treatment of this disease are currently lacking.

The disclosures of all publications, patents, patent applications and published patent applications referred to herein are hereby incorporated herein by reference in their entirety.

### BRIEF SUMMARY OF THE APPLICATION

The present application in one aspect provides a chimeric peptide comprising a blocking peptide connected to a stabilizing peptide, wherein the blocking peptide specifically blocks interaction between spike glycoprotein (“SPIKE”) and Angiotensin-converting enzyme 2 (“ACE2”), and wherein the stabilizing peptide stabilizes secondary or tertiary structure of the blocking peptide. In some embodiments, the blocking peptide comprises a loop sequence within the receptor-binding domain (RBD) of SPIKE. In some embodiments, the loop sequence has a length of no more than about 20 amino acids. In some embodiments, the loop sequence has a length of about 7 amino acids to about 18 amino acids.

In some embodiments according to any of the chimeric peptides described above, the blocking peptide comprises a lysine (K) at the C-terminus.

In some embodiments according to any of the chimeric peptides described above, the loop sequence is selected from the group consisting of SEQ ID NOs: 1-11 and 42-46. In some embodiments, the loop sequence is selected from the group consisting of SEQ ID NOs: 1, 6, 8-11, 42 and 45.

In some embodiments according to any of the chimeric peptides described above, the blocking peptide comprises a sequence derived from a sequence within the extracellular domain of ACE2. In some embodiments, the blocking peptide comprises a sequence selected from the group consisting of SEQ ID NOs: 23-31 and 47-52. In some embodiments, the blocking peptide comprises a sequence selected from the group consisting of SEQ ID NOs: 23, 24, 26-28, 31 and 47-52.

In some embodiments according to any of the chimeric peptides described above, the loop sequence is cyclic.

In some embodiments according to any of the chimeric peptides described above, the stabilizing peptide is connected to the C-terminus of the blocking peptide.

In some embodiments according to any of the chimeric peptides described above, the stabilizing peptide is connected to the N-terminus of the blocking peptide.

In some embodiments according to any of the chimeric peptides described above, the stabilizing peptide has a length of about 12 amino acids to about 30 amino acids.

In some embodiments according to any of the chimeric peptides described above, the blocking peptide and the stabilizing peptide each comprises a sequence derived from ACE2. In some embodiments, the stabilizing peptide comprises a sequence set forth in SEQ ID NO: 49 or 50.

In some embodiments according to any of the chimeric peptides described above, the stabilizing peptide comprises an amphipathic helix structure. In some embodiments, the stabilizing peptide comprises an ADGN-100 peptide or a VEPEP-6 peptide. In some embodiments, the stabilizing peptide comprises a sequence set forth in any one of SEQ ID NOs: 53-107. In some embodiments, the stabilizing peptide comprises a sequence set forth in SEQ ID NO: 55 or 97.

In some embodiments according to any of the chimeric peptides described above, the blocking peptide and the stabilizing peptide are connected via a linker. In some embodiments, linker is selected from the group consisting of a proline, a polyglycine linker moiety, a PEG moiety, Aun, Ava, and Ahx. In some embodiments, the PEG moiety consists of about two to about seven ethylene glycol units.

In some embodiments according to any of the chimeric peptides described above, the chimeric peptide comprises the amino acid sequence of any one of SEQ ID NOs: 12-22, 27, 28, and 31-41. In some embodiments, the chimeric peptide comprises the amino acid sequence selected from the group consisting of SEQ ID NOs: 12, 17, 19-22, 27, 28, 31-33, 35, and 38-40.

The present application in another aspect provides a non-naturally occurring peptide comprising the amino acid sequence of any of SEQ ID NOs: 12-22, 24-41, and 151-160. In some embodiments, the peptide comprises the amino acid sequence of any of SEQ ID NOs: 12, 17, 19-22, 24, 26-28, 31-33, 35, 38-40, 151, 156, and 158-160.

The present application in another aspect provides a siRNA comprising a nucleic acid sequence selected from the group consisting of SEQ ID NOs: 161-180. In some embodiments, the siRNA comprises a nucleic acid sequence set forth in SEQ ID NO: 163, 166 or 168, or 170.

The present application in another aspect provides a complex comprising a) a cargo comprising any of the peptides or siRNAs described above, and b) a second peptide, wherein the peptide or siRNA is complexed with the second peptide. In some embodiments, the second peptide is a cell penetrating peptide selected from the group consisting of CADY, PEP-1 peptides, PEP-2 peptides, PEP-3 peptides, LNCOV peptides, VEPEP-3 peptides, VEPEP-6 peptides, VEPEP-9 peptides, and ADGN-100 peptides. In some embodiments, the molar ratio of the second peptide to the peptide or siRNA is between about 1:1 and about 80:1. In some embodiments, the molar ratio of the second peptide to the peptide is between about 2:1 to about 10:1. In some embodiments, the molar ratio of the second peptide to the siRNA is between about 5:1 to about 50:1. In some embodiments, the complex comprises a) the chimeric peptide of any of the claims 1-25, the peptide of claim 26 or 27, b) A siRNA of claim 28 or claim 29. In some embodiments, the siRNA comprises a nucleic acid sequence set forth in SEQ ID NO: 166. In some embodiments, the complex comprises a chimeric peptide comprising the amino acid sequence set forth in SEQ ID NO: 17 or 33.

The present application in another aspect provides a nanoparticle comprising any of the complexes described above. In some embodiments, the nanoparticle has a diameter of no more than about 100 nm. In some embodiments, the nanoparticle has a diameter of about 40 to about 60 nm.

The present application in another aspect provides a pharmaceutical composition comprising a) any of the peptides, siRNAs, complexes, or nanoparticles described above, and b) a pharmaceutically acceptable carrier. In some embodiments, the composition comprises two or more complexes or nanoparticles, wherein the two or more complexes or nanoparticles comprise different cargos.

The present application in another aspect provides a method of preparing any of the complexes or nanoparticles described above, comprising combining the cargo with the second peptide.

The present application in another aspect provides a method of treating a SARS-COV-2 infection in an individual, comprising administering an effective amount of any of the pharmaceutical compositions described above. In some embodiments, the pharmaceutical composition is administered via nebulization or local lung or nasal delivery. In some embodiments, the individual is a human.

### BRIEF DESCRIPTION OF THE DRAWINGS

FIGS. 1A-1D show the overall structure of the SARS-CoV-2 receptor-binding domain (RBD) bound to ACE2 The structure is based on the crystal structure of the RBD of the spike protein of SARS-CoV-2 bound to the cell receptor ACE2 reported by Lan et al. (Nature volume 581, pages 215-220 (2020)). In FIG. 1A and FIG. 1B, the part of the SARS-CoV-2 RBM domain interacting with N-terminal helix of ACE2 protein is in light grey. In FIGS. 1C and 1D, the ACE2 domain interacting with the SARS-CoV-2 RBD domain is highlighted. The α1 and α2 helices contacting the RBM domain of the SARS-CoV-2 RBD are in light grey.

FIGS. 2A-2E show the structural organization of selected peptide inhibitors. Structural dynamic and stability analysis was determined for each helical peptides using Peplook-Zultim program. The ADGN peptide is in black and the inhibitory peptide is in light grey. The residues forming the major interaction are reported in sticks.

FIGS. 3A-3B show peptide inhibitor screening on the ACE2:SARS-CoV-2 Spike Inhibitor Assay. The different peptides were evaluated in a range of concentrations (0.1 nM-10 μM) as a free peptide (FIG. 3A) or within nanoparticle complex formed with ADGN-106 at 5/1 molar ratio (FIG. 3B). Results correspond to an average of 3 separate experiments.

FIGS. 4A-4B show peptide inhibitor screening on the SARS-CoV-2 Spike:ACE2 Inhibitor Assay. The different peptides were evaluated in a range of concentrations (0.1 nM-10 μM) as a free peptide (FIG. 4A) or within nanoparticle complex formed with ADGN-106 at 5/1 molar ratio (FIG. 4B). Results correspond to an average of 3 separate experiments.

FIGS. 5A-5D show the evaluation of peptides inhibitor on SARS-CoV-2 virus infection. Cells were plated in 96 wells one day prior to infection. Peptide solutions were made in DMSO/water (2%) and diluted from stock of 5 mM. Peptides with different dilution concentrations were either directly added to monolayer Vero-E6 cells (FIG. 5C) immediately prior to virus infection or mixed with SARS-CoV-2 for 30 min and then added to monolayer Vero-E6 cells (FIG. 5A). ADGN-106, hydroxy-chloroquine and buffer were used as control and all treatments were performed in triplicate. Percentage of inhibition and cytopathic effect were measured after 72 hr. The cellular toxicity of the different peptides was analyzed on Vero-6, cells using CellTiter-Glo assays (FIGS. 5B & 5D).

FIGS. 6A-6B show the evaluation of siRNAs targeting SARS-CoV-2 Nucleocapsid gene. H1299 lung epithelia cells were transfected with pcDNA3.1(+)-N-eGFP-NP plasmid encoding for SARS-COV-2 nucleocapside-tagged with eGFP. Cells were then treated with siRNAs (1 nM to 200 nM) complexed with ADGN-100 at molar ratio 1/20. siRNA targeting eGFP and scr-siRNA were used as positive and negative controls, respectively. Level of Nucleocapside-eGFP protein was evaluated 48 hr post transfection (FIG. 6A) and toxicity was determined using CellTiter Glow kits on GlowMax (FIG. 6B).

FIGS. 7A-7B show the evaluation of siRNAs targeting SARS-CoV-2 ORF3a gene. H1299 lung epithelia cells were transfected with pcDNA3.1(+)-N-eGFP-ORF3a plasmid encoding for SARS-COV-2 ORF3a-tagged with eGFP. Cells were then treated with siRNAs (1 nM to 200 nM) complexed with ADGN-100 at molar ratio 1/20. siRNA targeting eGFP and scr-siRNA were used as positive and negative controls, respectively Level of ORF3A-eGFP protein was evaluated 48 hr post transfection (FIG. 7A) and toxicity was determined using CellTiter Glow kits on GlowMax (FIG. 7B).

FIGS. 8A-8B show the evaluation of siRNAs targeting SARS-CoV-2 ORF8 gene. H1299 lung epithelia cells were transfected with pcDNA3.1(+)-N-eGFP-ORF8 plasmid encoding for SARS-COV-2 ORF8-tagged with eGFP. Cells were then treated with siRNAs (1 nM to 200 nM) complexed with ADGN-100 at molar ratio 1/20. siRNA targeting eGFP and scr-siRNA were used as positive and negative controls, respectively Level of ORF8-eGFP protein was evaluated 48 hr post transfection (FIG. 8A) and toxicity was determined using CellTiter Glow kits on GlowMax (FIG. 8B).

FIGS. 9A-9B show the evaluation of siRNA on SARS-CoV 2 virus infection. Cells were plated in 96 wells one day prior to infection. siRNAs were complexed with ADGN-100 at molar ratio 1/20. siRNA/ADGN-100 with different dilution concentrations were either directly added to monolayer Vero-E6 cells (FIG. 9A) immediately prior to virus infection. ADGN-100, and buffer were used as control and all treatments were performed in triplicate. Percentage of inhibition and cytopathic effect were measured after 72 hr. The cellular toxicity of the different siRNA/ADGN complexes was analyzed on Vero-6, cells using CellTiter-Glo assays (FIG. 9B).

FIG. 10 shows the evaluation of siRNA/inhibitor peptides on SARS-CoV 2 virus infection. Cells were plated in 96 wells one day prior to infection. siRNA solutions were made in DMSO/water (2%) and diluted from stock of 5 mM. siRNA/ADGN-100 complexes and LNCOV-15 or LNCOV-18 peptides with different dilution concentrations were directly added to monolayer Vero-E6 cells prior to virus infection. ADGN-100, and buffer were used as control and all treatments were performed in triplicate. Percentage of inhibition and cytopathic effect were measured after 72 hr.

FIG. 11A shows the evaluation of DIVC-6 siRNA targeting SARS-CoV-2 Nucleocapsid gene in complex with LNCOV peptides. H1299 lung epithelia cells were transfected with pcDNA3.1(+)-N-eGFP-NP plasmid encoding for SARS-COV-2 nucleocapside-tagged with eGFP. Cells were then treated with siRNAs (1 nM to 200 nM) complexed with ADGN-100; LNCOV-15 and LNCOV-18 at molar ratio 1/20. scr-siRNA were used was positive and negative controls, respectively. Level of Nucleocapside-eGFP protein was evaluated 48 hr post transfection.

FIG. 11B shows VEPEP-9 peptides with secondary structure indicated. “H” stands for “helix” and “t” stands for turn. FIGS. 12A-D show the impact of peptide inhibitor on the ACE2:SARS-CoV-2 Spike Inhibitor Assay. The different peptides were evaluated in a range of concentrations (0.1 nM-10 μM). Peptide inhibition was evaluated on the four different SARS-COV-2 Spike protein variants harboring the major mutations including Alpha (FIG.-12A), Beta (FIG.-12B), Gamma (FIG.-12C) and Delta (FIG.-12D) variants molar ratio (FIG. 3B). Results correspond to an average of 3 separate experiments.

FIGS. 12A-12E show the impact of peptide inhibitor on the ACE2:SARS-CoV-2 Spike Inhibitor Assay. The different peptides were evaluated in a range of concentrations (0.1 nM-10 μM). Peptide inhibition was evaluated on the four different SARS-COV-2 Spike protein variants harboring the major mutations including Alpha (FIG. 12A), Beta (FIG. 12B), Gamma (FIG. 12C), Delta (FIG. 12D) and Epsilon (FIG. 12E) variants. Results correspond to an average of 3 separate experiments.

FIGS. 13A-13C show the evaluation of peptides inhibitor on SARS-CoV-2 variant virus infection. Cells were plated in 96 wells one day prior to infection. The antiviral assay was performed on Vero E6 cells. Cells are infected in triplicate with SARS-CoV-2 Alpha (FIG. 13A) or Beta (FIG. 13B) or Delta (FIG. 13c) variant at MOI 0.001 by incubation for 1 hour in mediums containing either Seq17, Seq28 or Seq33 peptides (concentration ranging from 10 nM to 1 μM) or 6 μM of remdesivir (positive control RMD), or no antiviral molecule (negative control; “T−”). Peptide solutions were made in DMSO/water (2%) and diluted from stock of 5 mM. Supernatants were harvested 24 hours post-infection and viral titers were determined by the TCID50 method on Vero E6 cells and calculated by the Spearman & Kärber algorithm.

FIGS. 14A-14C show the evaluation of siRNA/inhibitor peptides on SARS-CoV2 variant virus infection. Cells were plated in 96 wells one day prior to infection. siRNA solutions were made in DMSO/water (2%) and diluted from stock of 5 mM. siRNA/ADGN-100 complexes and LNCOV-15, LNCOV-20 or LNCOV-18 peptides with different dilution concentrations were directly added to monolayer Vero-E6 cells prior to virus infection. 6 μM of remdesivir (positive control RMD), or no antiviral molecule (negative control; “T-”). Peptide solutions were made in DMSO/water (2%) and diluted from stock of 5 mM. Supernatants were harvested 24 hours post-infection and viral titers were determined by the TCID50 method on Vero E6 cells and calculated by the Spearman & Kärber algorithm.

FIGS. 15A-15E show in vivo lung biodistribution of peptides and peptide/siRNA inhibitor of SARS-COV-2. The study was performed in healthy 4 weeks old male C57BL/6J mice. Single dose of peptides or peptide/siRNA complexes were administrated via intratracheal instillation (200 μg). The fluorescence was evaluated by non-invasive in vivo whole-body fluorescence imaging at T0, 1 h, 2 h, 6 h, 24 h, 48 h and 72 hr post instillation (FIG. 15A). Ex vivo fluorescence signals were observed on organs at 6 hr, 12 hr, 48 hr and 72 hr post instillation. Semi-quantitative data were obtained from the fluorescence images by using the Living image software.

FIGS. 16A-16B show confocal microscopy analysis of lung treated with of peptides and peptide/siRNA inhibitor of SARS-COV-2. The study was performed in healthy 4 weeks old male C57BL/6J mice. Single dose of peptides or peptide/siRNA complexes were administrated via intratracheal instillation (200 μg). Lungs were collected at 4 hr, 24 hr and 72 hr post administration, fixed in formaldehyde 4% in Glucose 5%. Tissues were stained with Mito tracker red and nucleus with Hoesch.

FIG. 17 show quantitative confocal microscopy analysis of lung treated with of peptides and peptide/siRNA inhibitor of SARS-COV-2. The study was performed in healthy 4 weeks old male C57BL/6J mice. Single dose of peptides or peptide/siRNA complexes were administrated via intratracheal instillation (200 μg). Lungs were collected at 4 hr, 24 hr and 72 hr post administration, fixed in formaldehyde 4% in Glucose 5%. Tissues were stained with Mito tracker red and nucleus with Hoesch. One-way ANOVA with multiple comparisons on log-transformed data, where ***P=0.0005, **P=0.001 and *P=0.0011; NS, not significant.

FIGS. 18A-18G show characterization of SARS-COV2 inhibitor in vivo, Body and organ weight analysis. The body weight of the mice were recorded before intratracheal instillation (day 0), at day1, day 2 and before being sacrificed (day 3). The lung, liver, heart, kidney and brain were collected at day 3 and organ index were determined.

FIGS. 19A-19C show bronchoalveolar lavage analysis after peptides and peptide/siRNA complex treatments. BAL were performed 2 days after instillation. The percentage of the cells, the total protein and level of LDH in the BAL were analyzed. Results were compared to saline buffer used as negative control.

FIGS. 20A-20B show AST and ALT blood analysis after peptides and peptide/siRNA complex treatments. Single dose of peptides or peptide/siRNA complexes were administrated via intratracheal instillation (200 μg). Saline solution was used as negative control. AST and ALT levels in the plasma were assayed 2 days after instillation.

### DETAILED DESCRIPTION OF THE APPLICATION

The present application in one aspect provides novel inhibitory peptides (i.e. blocking peptides) that block the interaction between spike glycoprotein (SPIKE) of coronavirus (such as SARS-CoV-2) and ACE2 expressed on various cell types (such as lung cells) of an individual (such as human). As shown in the Example section, exemplary inhibitory peptides effectively block the interaction between SPIKE and ACE2 both in vitro and on infected cells.

In some embodiments, the inhibitory peptide is a chimeric peptide that comprises a blocking peptide connected with a stabilizing peptide, wherein the stabilizing peptide stabilizes secondary or tertiary structure of the blocking peptide. Without being bound to the theory, it was observed that peptides adopting an amphipathic helix structure stabilizes helical blocking peptides, which involve mainly aromatic residues (e.g., tryptophan group residues) and electrostatic interactions located on the same side of the helix. It was also observed that cyclization of blocking peptides that have loop sequences stabilizes the blocking peptides, and fusing the cyclized blocking peptide with a stabilizing peptide further stabilizes the blocking peptide. As demonstrated in the examples, exemplary peptides (such as peptides comprising a sequence set forth in SEQ ID NO: 17, 28, or 33) exhibit potent anti-viral effects and capability to penetrate lung.

The present application in another aspect provides novel nucleic acids (siRNAs) that targets SARS-CoV-2. In some embodiments, the siRNA comprises a nucleic acid sequence set forth in SEQ ID NO: 163, 166 or 170.

The present application in another aspect provides complexes and nanoparticles comprising any of the inhibitory peptides or nucleic acids described herein. In some embodiments, the complex or nanoparticle comprises a second peptide, wherein the second peptide is complexed with any one or more of the inhibitory peptides and/or nucleic acids (such as siRNAs). It was observed that that inhibitory peptide and/or silencing RNAs based complexes and nanoparticles neutralize SARS-CoV-2 cellular entry and prevent virus production by the delivery of silencing RNA. In summary, the inhibitory peptides, nucleic acids, complexes, nanoparticles described herein provide simple and efficient therapeutics against the COVID-19 disease.

The present application also provides methods of treating COVID-19 by administering the chimeric peptides, peptides, siRNAs, complexes, and/or nanoparticles described herein.

### I. Definitions

The terms “non-naturally occurring,” “synthetic,” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

“Polynucleotide,” or “nucleic acid,” as used interchangeably herein, refers to polymers of nucleotides of any length, and includes DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. The term “nucleic acid” as used herein refers to a polymer containing at least two deoxyribonucleotides or ribonucleotides in either single- or double-stranded form and includes DNA and RNA. DNA may be in the form of, e.g., antisense molecules, plasmid DNA, pre-condensed DNA, a PCR product, vectors (PAC, BAC, YAC, artificial chromosomes), expression cassettes, chimeric sequences, chromosomal DNA, or derivatives and combinations of these groups. RNA may be in the form of siRNA, asymmetrical interfering RNA (aiRNA), microRNA (miRNA), mRNA, tRNA, rRNA, RNA, viral RNA (vRNA), and combinations thereof. Nucleic acids include nucleic acids containing known nucleotide analogs or modified backbone residues or linkages, including for example locked nucleic acid (LNA), unlocked nucleic acid (UNA), and zip nucleic acid (ZNA), which can be synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer e al., Nucleic Acid Res., 19:5081 (1991); Ohtsuka et a., j. Biol. Chern., 260:2605-2608 (1985); Rossolini et al., Mol. Cell. Probes, 8:91-98 (1994)). “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylases, and alkylhalides. “Oligonucleotide,” as used herein, generally refers to short, generally synthetic polynucleotides that are generally, but not necessarily, less than about 200 nucleotides in length. The terms “oligonucleotide” and “polynucleotide” are not mutually exclusive. The description above for polynucleotides is equally and fully applicable to oligonucleotides.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating” is an approach for obtaining beneficial or desired results, including clinical results. For purposes of this invention, beneficial or desired clinical results include, but are not limited to, one or more of the following: alleviating one or more symptoms resulting from the disease (e.g., decreasing viral load, or slow the increase of viral load as compared to a non-treated individual also infected with the virus), diminishing the extent of the disease (e.g., decreasing the course of the infection, such as decreasing the onset of the infection, decreasing the days that the individual is in intensive care unit (ICU) or ventilator), stabilizing the disease (e.g., preventing or delaying the worsening of the disease), preventing or delaying the spread of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of the disease (such as, for example, respiratory symptoms in a coronavirus infection). The methods of the invention contemplate any one or more of these aspects of treatment.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

It is understood that embodiments of the invention described herein include “consisting” and/or “consisting essentially of” embodiments.

As used herein, the singular form “a”, “an”, and “the” includes plural references unless indicated otherwise.

Reference to “about” a value or parameter herein includes (and describes) embodiments that are directed to that value or parameter per se. For example, description referring to “about X” includes description of “X.”

The compositions and methods of the present application may comprise, consist of, or consist essentially of the essential elements and limitations of the application described herein, as well as any additional or optional ingredients, components, or limitations described herein or otherwise useful.

Unless otherwise noted, technical terms are used according to conventional usage.

### Chimeric Peptides

The present application provides chimeric peptides comprising a blocking peptide connected to a stabilizing peptide, wherein the blocking peptide specifically blocks interaction between SPIKE and ACE2, and wherein the stabilizing peptide stabilizes secondary or tertiary structure of the blocking peptide.

In some embodiments, the chimeric peptide has a length of about 5 to about 100 amino acids (such as about 10 to about 80 amino acids, about 10 to about 70 amino acids, about 10 to about 60 amino acids, about 10 to about 50 amino acids).

In some embodiments, there is provided a chimeric peptide comprising a blocking peptide connected to a stabilizing peptide, wherein the blocking peptide specifically blocks interaction between SPIKE and ACE2 and comprises a loop sequence within the receptor-binding domain (RBD) of SPIKE, and wherein the stabilizing peptide comprises an amphipathic helix. In some embodiments, the stabilizing peptide is selected from the group consisting of CADY, VEPEP-106 peptide, ADGN-100 peptide, and VEPEP-109 peptide. In some embodiments, the stabilizing peptide is connected to the C-terminus of the blocking peptide. In some embodiments, the loop sequence has a length of no more than about 20 amino acids (such as about 7-18 amino acids). In some embodiments, the blocking peptide comprises a lysine (K) at the C-terminus. In some embodiments, the loop sequence is cyclic. In some embodiments, the loop sequence is selected from the group consisting of SEQ ID NOs: 1-11 and 42-46. In some embodiments, the loop sequence is selected from the group consisting of SEQ ID NOs: 1, 6, 8-11, 42 and 45. In some embodiments, the blocking peptide and the stabilizing peptide are connected via a linker. In some embodiments, the linker is selected from the group consisting of a proline, a polyglycine linker moiety, a PEG moiety, Aun, Ava, and Ahx.

In some embodiments, there is provided a chimeric peptide comprising a blocking peptide connected to a stabilizing peptide, wherein the blocking peptide specifically blocks interaction between SPIKE and ACE2 and comprises a sequence derived from a sequence within the extracellular domain of ACE2, and wherein the stabilizing peptide comprises an amphipathic helix. In some embodiments, the stabilizing peptide is selected from the group consisting of CADY, VEPEP-106 peptide, ADGN-100 peptide, and VEPEP-109 peptide. In some embodiments, the stabilizing peptide is connected to the C-terminus of the blocking peptide. In some embodiments, the blocking peptide comprises a sequence selected from the group consisting of SEQ ID NOs: 23-31 and 47-52. In some embodiments, the blocking peptide comprises a sequence selected from the group consisting of SEQ ID NOs: 23, 24, 26-28, 31 and 47-52. In some embodiments, the blocking peptide and the stabilizing peptide are connected via a linker. In some embodiments, the linker is selected from the group consisting of a proline, a polyglycine linker moiety, a PEG moiety, Aun, Ava, and Ahx.

In some embodiments, there is provided a chimeric peptide comprising a blocking peptide connected to a stabilizing peptide, wherein the blocking peptide comprises a sequence selected from the group consisting of SEQ ID NOs: 1, 6, 8-11, 23, 24, 26-28, 31 42, 45 and 47-52, wherein the stabilizing peptide comprises an ADGN-100 peptide or a VEPEP-6 peptide. In some embodiments, the blocking peptide and the stabilizing peptide are connected via a linker. In some embodiments, the linker is selected from the group consisting of a proline, a polyglycine linker moiety, a PEG moiety, Aun, Ava, and Ahx.

In some embodiments, there is provided a chimeric peptide comprising a blocking peptide connected to a stabilizing peptide, wherein the blocking peptide comprises a sequence selected from the group consisting of SEQ ID NOs: 1, 6, and 8-11, wherein the stabilizing peptide comprises the amino acid sequence of SEQ ID NO: 55 or 97. In some embodiments, the blocking peptide is cyclic. In some embodiments, the blocking peptide and the stabilizing peptide are connected via a linker. In some embodiments, the linker is selected from the group consisting of a proline, a polyglycine linker moiety, a PEG moiety, Aun, Ava, and Ahx.

In some embodiments, there is provided a chimeric peptide comprising a blocking peptide connected to a stabilizing peptide, wherein the blocking peptide comprises a cyclic peptide selected from the group consisting of SEQ ID NOs: 151, 156, and 158-160, wherein the stabilizing peptide comprises the amino acid sequence of SEQ ID NO: 55 or 97. In some embodiments, the blocking peptide and the stabilizing peptide are connected via a linker. In some embodiments, the linker is selected from the group consisting of a proline, a polyglycine linker moiety, a PEG moiety, Aun, Ava, and Ahx.

In some embodiments, there is provided a chimeric peptide comprising a blocking peptide connected to a stabilizing peptide, wherein the blocking peptide comprises a sequence selected from the group consisting of SEQ ID NOs: 23, 24, 26-28, 31 and 47-52, wherein the stabilizing peptide comprises the amino acid sequence of SEQ ID NO: 55 or 97. In some embodiments, the blocking peptide comprises a sequence selected from the group consisting of SEQ ID NOs: 23, 24, 26-28 and 31. In some embodiments, the blocking peptide and the stabilizing peptide are connected via a linker. In some embodiments, the linker is selected from the group consisting of a proline, a polyglycine linker moiety, a PEG moiety, Aun, Ava, and Ahx.

In some embodiments, there is provided a chimeric peptide comprising an amino acid sequence set forth in SEQ ID NOs: 17, 20, 21, 27, 28, 33, 39, and 40.

### Blocking Peptide

The present application provides blocking peptides and chimeric peptides comprising a blocking peptide and a stabilizing peptide as described herein.

In some embodiments, the blocking peptide comprises a loop sequence within the receptor-binding domain (RBD) of SPIKE or a sequence derived from a sequence (such as a loop sequence, an alpha helix, a beta strand, or a combination thereof) within the extracellular domain of ACE2 (such as human ACE2). Loop sequence described herein is selected from the structure of the SPIKE or ACE protein and located between two secondary structure motifs that can be beta strand or alpha helix. The loop sequence can be cyclized.

In some embodiments, the loop sequence has a length of no more than about 50 amino acids (such as no more than about 40, 35, 30, 25, or 22 amino acids). In some embodiments, the loop sequence has a length of no more than about 20 amino acids (such as no more than about 18, 15, 12, or 10 amino acids).

In some embodiments, the loop sequence has a length of about 5-40 amino acids (such as about 5-30 amino acids, 5-25 amino acids, 5-20 amino acids). In some embodiments, the loop sequence has a length of about 7 amino acids to about 18 amino acids.

In some embodiments, the loop sequence is cyclic.

In some embodiments, the blocking peptide comprises a lysine (K) at the C-terminus. The lysine can be used to promote cyclization of the loop via lysine side-chain anchoring.

In some embodiments, the blocking peptide comprises a loop sequence within the receptor-binding domain (RBD) of SPIKE. In some embodiments, the loop sequence is within the receptor-binding motif (RBM) of SPIKE. In some embodiments, the loop sequence is within any of the RBM structure motif selected from the groups consisting of loop α4-β5, loop β5-β6, and loop β6-α5.

In some embodiments, the loop sequence is selected from the group consisting of SEQ ID NOs: 1-11 and 42-46. In some embodiments, the loop sequence is selected from the group consisting of SEQ ID NOs: 1, 6, 8-11, 42 and 45. In some embodiments, the blocking peptide comprises a sequence selected from the group consisting of SEQ ID NOs: 1, 6, and 8-11.

In some embodiments, the blocking peptide comprises a sequence derived from a sequence (such as a loop sequence, or an alpha helix motif) within the extracellular domain of ACE2 (such as human ACE2). In some embodiments, the blocking peptide comprises a sequence derived from a sequence within al helix of human ACE2. In some embodiments, the blocking peptide comprises a sequence derived from a sequence within α1 and α2 helices of human ACE2. In some embodiments, the blocking peptide comprises a sequence derived from a loop sequence between 33 and 34 strands of human ACE2.

In some embodiments, the sequence derived from a sequence within the extracellular domain of ACE2 is different from the sequence within the extracellular domain of ACE2 in one, two or three amino acids at the C-terminus of the sequence.

In some embodiments, the blocking peptide comprises a sequence within the extracellular domain of ACE2.

In some embodiments, the blocking peptide comprises a sequence selected from the group consisting of SEQ ID NOs: 23-31 and 47-52. In some embodiments, the blocking peptide comprises a sequence selected from the group consisting of SEQ ID NOs: 23, 24, 26-28, 31 and 47-52. In some embodiments, the blocking peptide comprises a sequence selected from the group consisting of SEQ ID NOs: 23, 24, 26-28 and 31.

In some embodiments, the blocking peptide comprises a cyclic peptide selected from any one of SEQ ID NOs: 151-160. In some embodiments, the blocking peptide comprises a cyclic peptide selected from the group consisting of SEQ ID NOs: 151, 156, and 158-160.

In some embodiments, the blocking peptide comprises a non-naturally occurring peptide selected from any one of SEQ ID NOs: 12-22 and 24-41.

In some embodiments, the blocking peptide comprises a sequence derived from α1 and α2 helices of ACE2 (e.g. human ACE2). In some embodiments, the blocking peptide comprises a first sequence derived from α1 helix of ACE2 (e.g., human ACE2) and a second sequence derived from α2 helix of ACE2 (e.g., human ACE2). In some embodiments, the first sequence comprises an amino acid sequence set forth in SEQ ID NO: 47 or 48, and the second sequence comprises an amino acid sequence set forth in SEQ ID NO: 49 or 50. In some embodiments, the first sequence comprises an amino acid sequence set forth in SEQ ID NO: 51, and the second sequence comprises an amino acid sequence set forth in SEQ ID NO: 52. In some embodiments, the second sequence is connected to the C-terminus of the first sequence. In some embodiments, the second sequence is connected to the N-terminus of the first sequence. In some embodiments, the first sequence and the second sequence are connected via a linker (such as any of the linkers described herein). In some embodiments, the linker is selected from the group consisting of a proline, a polyglycine linker moiety, a PEG moiety, Aun, Ava, and Ahx. In some embodiments, the blocking peptide comprises an amino acid sequence of any one of SEQ ID NOs: 27, 38, 39, 40.

In some embodiments, the blocking peptide described herein is stapled. “Stapled” as used herein refers to a chemical linkage between two residues in a peptide. In some embodiments, the blocking peptide is stapled, comprising a chemical linkage between two amino acids of the peptide. In some embodiments, the two amino acids linked by the chemical linkage are separated by 3 or 6 amino acids. In some embodiments, two amino acids linked by the chemical linkage are separated by 3 amino acids. In some embodiments, the two amino acids linked by the chemical linkage are separated by 6 amino acids. In some embodiments, each of the two amino acids linked by the chemical linkage is R or S. In some embodiments, each of the two amino acids linked by the chemical linkage is R. In some embodiments, each of the two amino acids linked by the chemical linkage is S. In some embodiments, one of the two amino acids linked by the chemical linkage is R and the other is S. In some embodiments, the chemical linkage is a hydrocarbon linkage.

In some embodiments, there is provided a blocking peptide comprising the amino acid sequence of any of SEQ ID NOs: 1-52 and 151-160, wherein at least one or more amino acid is a D-amino acid. In some embodiments, at least about 5%, 10%, 15%, 20%, 25%, 30%, 35%, 40%, 45%, 50%, 55%, 60%, 65%, 70%, 75%, 80%, 85%, 90%, or 95% of the amino acids in the blocking peptide is or are in the form of D-amino acid. In some embodiments, the blocking peptide is comprised of 100% D-amino acids. In some embodiments, at least a portion of the blocking peptide is in a cyclic form. In some embodiments, at least a portion of the blocking peptide has an α-helical structure. In some embodiments, the blocking peptide comprises an amino acid sequence of any one of SEQ ID NOs: 12-22, 24-41, and 151-160. In some embodiments, the blocking peptide comprises an amino acid sequence of any one of SEQ ID NOs: 151-160. In some embodiments, the blocking peptide comprises the amino acid sequence of any of SEQ ID NOs: 12, 17, 19-22, 24, 26-28, 31-33, 35, 38-40, 151, 156, and 158-160.

In some embodiments, there is provided a blocking peptide comprising the amino acid sequence of any of SEQ ID NOs: 1-22, 42-46 and 151-160. In some embodiments, at least a portion of the blocking peptide is in a cyclic form. In some embodiments, at least about 5%, 10%, 15%, 20%, 25%, 30%, 35%, 40%, 45%, 50%, 55%, 60%, 65%, 70%, 75%, 80%, 85%, 90%, or 95% of the amino acids in the blocking peptide is or are in the form of D-amino acid. In some embodiments, the blocking peptide is comprised of 100% D-amino acids. In some embodiments, the blocking peptide comprises a cyclic peptide selected from the group consisting of SEQ ID NOs: 151, 156, and 158-160.

In some embodiments, there is provided a blocking peptide comprising the amino acid sequence of any of SEQ ID NOs: 23-41 and 47-52. In some embodiments, at least a portion (e.g., about 20%, 30%, 40%, 50%, 60%, 70%, 80%, or 90%) of the blocking peptide has an α-helical structure. In some embodiments, at least about 5%, 10%, 15%, 20%, 25%, 30%, 35%, 40%, 45%, 50%, 55%, 60%, 65%, 70%, 75%, 80%, 85%, 90%, or 95%, or about 100% of the amino acids on the non-helical structure is or are in the form of D-amino acid. In some embodiments, at least about 5%, 10%, 15%, 20%, 25%, 30%, 35%, 40%, 45%, 50%, 55%, 60%, 65%, 70%, 75%, 80%, 85%, 90%, or 95%, or about 100% of the amino acids (e.g., the amino acids on the non-helical structure) comprise a retro-inverso peptide. In some embodiments, at least a portion of the blocking peptide (e.g., a portion of the non-helical structure) is stapled. In some embodiments, the blocking peptide is stapled, comprising a chemical linkage between two amino acids of the peptide. In some embodiments, the two amino acids linked by the chemical linkage are separated by 3 or 6 amino acids. In some embodiments, two amino acids linked by the chemical linkage are separated by 3 amino acids. In some embodiments, the two amino acids linked by the chemical linkage are separated by 6 amino acids. In some embodiments, each of the two amino acids linked by the chemical linkage is R or S. In some embodiments, each of the two amino acids linked by the chemical linkage is R. In some embodiments, each of the two amino acids linked by the chemical linkage is S. In some embodiments, one of the two amino acids linked by the chemical linkage is R and the other is S. In some embodiments, the chemical linkage is a hydrocarbon linkage.

In some embodiments, there is provided a blocking peptide (or a non-naturally occurring peptide) comprising the amino acid sequence of any of SEQ ID NOs: 12-22, 24-41, and 151-160. In some embodiments, the blocking peptide comprises the amino acid sequence of any of SEQ ID NOs: 12, 17, 19-22, 24, 26-28, 31-33, 35, 38-40, 151, 156, and 158-160. In some embodiments, the blocking peptide (or the non-occurring peptide) has a length of about 5 to about 100 amino acids (such as about 10 to about 80 amino acids, about 10 to about 70 amino acids, about 10 to about 60 amino acids, about 10 to about 50 amino acids).

The present application also provides any of the blocking peptides described herein connected to a second moiety. The second moiety stabilizes the structure (such as the secondary structure, e.g., a loop or a helical structure) of the blocking peptide. In some embodiments, the second moiety is a PEG moiety. In some embodiments, the second moiety is a lipid. In some embodiments, the second moiety is a nanoparticle. In some embodiments, the second moiety is an antibody. In some embodiments, at least a portion of the blocking peptide is in a cyclic form. In some embodiments, the blocking peptide comprises a cyclic peptide selected from the group consisting of SEQ ID NOs: 151-160 (e.g., SEQ ID NOs: 151, 156, and 158-160).

In some embodiments, the second moiety is a PEG moiety (such as any of the PEG moieties described herein). PEGylation of peptide provide various advantages, such as promoting secondary structure of alpha-helices, promoting drug delivery and in vivo circulation time of the conjugate. See for example, Hamed et al. (Biomacromolecules 2013, 14, 4053-4060), Hamley (Biomacromolecules 2014, 15, 1543-1559), and Lawrence et al., (Curr Opin Chem Biol. 2016 October; 34: 88-94). Methods of linking a PEG moiety to a peptide is known in the field, for example, such as those described in Hamed and Hamley. In some embodiments, the PEG moiety consisting of no more than about 7, 6, 5, 4, 3, or 2 units. In some embodiments, the PEG moiety consists of two or four units.

In some embodiments, the second moiety is an antibody that targets or neutralize a virus (e.g., a coronavirus, e.g., SARS-CoV-2). In some embodiments, the antibody is a nanobody. Exemplary antibodies can be found in, for example, Wang et al. (Nature Communications, volume 11, Article number: 2251 (2020)), and Pinto et al., (Nature volume 583, pages 290-295(2020)).

In some embodiments, the second moiety is a nanoparticle. In some embodiments, the nanoparticle is a gold nanoparticle (e.g., Au-nanoparticle). General principles and methods of connecting a peptide to a nanoparticle is known and appreciated in the field, for example, as described in Brancolini et al. (Current Opinion in Colloid & Interface Science 2019, 41:86-94).

In some embodiments, the second moiety is a lipid. Peptide lipidation promotes pharmacokinetic and pharmacodynamics profiles of peptide-based drugs. See for example, Kowalczyk et al. (Adv Exp Med Biol. 2017; 1030:185-227) and Ward et al. (Molecular Metabolism 2(2013) 468-479). Methods of connecting the lipid to a peptide is known in the field, for example, such as those described in Kowalcyzk and Ward. In some embodiments, the lipid is an acylating agent. In some embodiments, the blocking peptide is acylated. In some embodiments, the blocking peptide is acylated with a C8 or C16 fatty acid. In some embodiments, the C8 or C16 fatty acid is in a linear hydrocarbon chain.

In some embodiments, the second moiety is connected to the N-terminus of the peptide. In some embodiments, the second moiety is connected to the C-terminus of the peptide. In some embodiments, the second moiety is added to one or more amino acids (such as a lipid added to cysteine, serine, threonine, or lysine) within the peptide.

In some embodiments, the blocking peptide and the second moiety is connected via a linker (such as any of the linkers described herein).

### Stabilizing Peptide

Stabilizing peptides in some embodiments comprises an amphipathic alpha helix (or amphipathic helix) structure. Amphipathic helices are peptide sequences that fold into alpha helical structure upon contact with a polar/non-polar interface. Amphipathic helices, a secondary feature found in many proteins, are defined by their structure and by the segregation of hydrophobic and polar residues between two faces of the helix. This segregation allows AHs to adsorb at polar-apolar interfaces.

In some embodiments, the stabilizing peptide is connected to the C-terminus of the blocking peptide. In some embodiments, the stabilizing peptide is connected to the N-terminus of the blocking peptide. In some embodiments, the stabilizing peptide is connected to the blocking peptide via a linker (such as any of the linkers described herein).

In some embodiments, the stabilizing peptide has a length of about 8 amino acids to about 50 amino acids (such as about 10 to about 40 amino acids, such as about 12 to about 30 amino acids).

In some embodiments, the stabilizing peptide comprises a single core helical motif. In some embodiments, the stabilizing peptide comprises two or more helical motifs. Exemplary stabilizing peptides comprise CADY (e.g., SEQ ID NO: 150), VEPEP-106 peptide, ADGN-100 peptide, and VEPEP-109 peptide. All four peptides adopt a secondary amphipathic helical conformation within membrane-mimicking environments, exposing Trp-groups on one side, charged residues on the other and hydrophobic residues on yet another. CADY and VEPEP-6 adopt the same secondary structure, with helical motif in the core, C- and N-terminus of the peptide (Table 1). In contrast ADGN-100 and VEPEP-9 peptides contains a single core helical motif, which is longer in VEPEP-9 and in agreement with Konate et al 2010 (Biochemistry), Crowlet et al 2014 (BBA).

TABLE 1

| Peptide | Secondary structure prediction |
|---|---|
| ADGN-100 | KWRSAGWRWRLWRVRSWSR (SEQ ID NO: 61) |
| | hhhhhhhhhhhhhh |
| CADY | GLWRALWRLLRSLWRLLWKV (SEQ ID NO: 150) |
| | hhh hhhhh hhhh |
| VEPEP-6 | LWRALWRLWRSLWRLLWKA (SEQ ID NO: 92) |
| | hhh hhhhh hhhh |
| VEPEP-9 | LRWWLRWASRWFSRWAWWR (SEQ ID NO: 120) |
| | hhhhhhhhhhhhhhhh |

h: helix motif

In some embodiments, the stabilizing peptide comprises an ADGN-100 peptide or a VEPEP-6 peptide.

In some embodiments, the stabilizing peptide is a retro-inverso peptide (e.g., a peptide made up of D-amino acids in a reversed sequence and, when extended, assumes a side chain topology similar to that of its parent molecule but with inverted amide peptide bonds) of any of the stabilizing peptides described herein.

In some embodiments, the stabilizing peptide described herein (e.g., CADY, VEPEP-3 peptide, VEPEP-4 peptide, VEPEP-5 peptide, VEPEP-6 peptide, VEPEP-9 peptide, or ADGN-100 peptide) is stapled. In some embodiments, the stabilizing peptide is stapled, comprising a chemical linkage between two amino acids of the peptide. In some embodiments, the two amino acids linked by the chemical linkage are separated by 3 or 6 amino acids. In some embodiments, two amino acids linked by the chemical linkage are separated by 3 amino acids. In some embodiments, the two amino acids linked by the chemical linkage are separated by 6 amino acids. In some embodiments, each of the two amino acids linked by the chemical linkage is R or S. In some embodiments, each of the two amino acids linked by the chemical linkage is R. In some embodiments, each of the two amino acids linked by the chemical linkage is S. In some embodiments, one of the two amino acids linked by the chemical linkage is R and the other is S. In some embodiments, the chemical linkage is a hydrocarbon linkage.

In some embodiments, the stabilizing peptide comprises a sequence derived from ACE2 (e.g., human ACE2). In some embodiments, the blocking peptide and the stabilizing peptide each comprises a sequence derived from ACE2 (e.g., human ACE2). In some embodiments, the stabilizing peptide comprises a sequence set forth in SEQ ID NO: 49 or 50. In some embodiments, the stabilizing peptide is connected to N-terminus of the blocking peptide. In some embodiments, the stabilizing peptide is connected to C-terminus of the blocking peptide. In some embodiments, the stabilizing peptide is connected to the blocking peptide via a linker (such as any of the linkers described herein).

In some embodiments, the stabilizing peptide comprises an amino acid sequence of any one of SEQ ID NOs: 49, 50, and 53-150.

### ADGN-100 Peptides

In some embodiments, the ADGN-100 peptides described herein comprises a core motif, wherein the core motif is the amino acid sequence RWRLWRX₁X₂X₃X₄SR (SEQ ID NO: 53), and wherein X₁ is V or S, X₂ is R, V, or A, X₃ is S or L, and X₄ is W or Y. In some embodiments, the peptide adopts an α-helical structure throughout more than about 50% (such as more than about any of 55%, 60%, 70%, 75%, 80%, 85%, 90%, and 95%) of its length. Exemplary ADGN-100 peptides are described in, for example, US20190002499A1, EP3237436, and WO2016/102687, which are incorporated herein by reference in their entirety.

In some embodiments, the helical structure is configured such that at least 2 (such as at least any of 3, 4, 5, 6, 7, or 8) of the S and R residues are on one side of the helical structure and at least 1 (such as at least any of 2, 3, 4, or 5) of the W residues are on the opposite side of the helical structure. In some embodiments, a majority of the S and R residues are on one side of the helical structure and a majority of the W residues are on the opposite side of the helical structure. In some embodiments, all of the S and R residues are on one side of the helical structure and all of the W residues are on the opposite side of the helical structure. In some embodiments, the helical structure is configured such that a patch of electrostatic contacts is formed on one side of the helical structure and a patch of hydrophobic contacts is formed on the other side of the helical structure. In some embodiments, the peptide adopts a single helix in the presence of a cargo molecule.

In some embodiments, the ADGN-100 peptide comprises a sequence set forth in any one of SEQ ID NOs: 53-79.

In some embodiments, the ADGN-100 cell-penetrating peptide comprises the amino acid sequence RSX₁X₂X₃RWRLWRX₄X₅X₆X₇SR (SEQ ID NO: 54), wherein X₁ is A or V, X₂ is G or L, X₃ is W or Y, X₄ is V or S, X₅ is R, V, or A, X₆ is S or L, and X₇ is W or Y. In some embodiments, the ADGN-100 peptide comprises the amino acid sequence RSAGWRWRLWRVRSWSR (SEQ ID NO: 55), RSALYRWRLWRVRSWSR (SEQ ID NO: 56), RSALYRWRLWRSRSWSR (SEQ ID NO: 57), or RSALYRWRLWRSALYSR (SEQ ID NO: 58). In some embodiments, the ADGN-100 peptide comprises the amino acid sequence RSAGWRWRLWRVRSWSR (SEQ ID NO: 55).

In some embodiments, the ADGN-100 cell-penetrating peptide comprises X₁KWRSX₂X₃X₄RWRLWRX₅X₆X₇X₈SR (SEQ ID NO: 59), wherein X₁ is any amino acid or none, and X₂-X₈ are any amino acid. In some embodiments, the ADGN-100 cell-penetrating peptide comprises X₁KWRSX₂X₃X₄RWRLWRX₅X₆X₇X₈SR, wherein X₁ is RA, S, or none, X₂ is A or V, X₃ is G or L, X₄ is W or Y, X₅ is V or S, X₆ is R, V, or A, X₇ is S or L, and X₈ is W or Y. In some embodiments, the ADGN-100 cell-penetrating peptide comprises the amino acid sequence of any one of SEQ ID NOs: 61-64.

In some embodiments, the ADGN-100 peptides described herein comprises a core motif, wherein the core motif is the amino acid sequence RWRLWRWSR (SEQ ID NO: 65).

In some embodiments, the ADGN-100 peptide is a retro-inverso peptide (e.g., a peptide made up of D-amino acids in a reversed sequence and, when extended, assumes a side chain topology similar to that of its parent molecule but with inverted amide peptide bonds). In some embodiments, the retro-inverso peptide comprises a sequence of SEQ ID NO: 66 or 67.

In some embodiments, the ADGN-100 peptide comprises two residues separated by three or six residues that are linked by a hydrocarbon linkage. In some embodiments, this linkage increases the stability of the chimeric peptide. In some embodiments, the ADGN-100 peptide comprises the amino acid sequence RSsAGWRsWRLWRVRSWSR (SEQ ID NO: 68), RsSAGWRWRsLWRVRSWSR (SEQ ID NO: 69), RSAGWRsWRLWRVRsSWSR (SEQ ID NO: 70), RSsALYRsWRLWRSRSWSR (SEQ ID NO: 71), RsSALYRWRsLWRSRSWSR (SEQ ID NO: 72), RSALYRsWRLWRSRsSWSR (SEQ ID NO: 73), RSALYRWRsLWRSsRSWSR (SEQ ID NO: 74), RSALYRWRLWRSsRSWSsR (SEQ ID NO: 75), RsSALYRWRsLWRSALYSR (SEQ ID NO: 76), RSsALYRsWRLWRSALYSR (SEQ ID NO: 77), RSALYRWRsLWRSsALYSR (SEQ ID NO: 78), or RSALYRWRLWRSsALYSsR (SEQ ID NO: 79), wherein the residues marked with a subscript “S” are linked by a hydrocarbon linkage.

### VEPEP-6 Peptides

In some embodiments, the stabilizing peptide is a VEPEP-6 peptide (also referred to as an ADGN-106 peptide or a VEPEP-106 peptide). VEPEP-6 peptides described herein are secondary amphipathic peptides. They are highly versatile and show a strong structural polymorphism. VEPEP-6 are unfolded in solution as a free form and adopted an alpha helical conformation in the presence of lipid or artificial cellular membranes as well as in the presence of cargos (such as siRNA/single stranded and double stranded oligonucleotides). See US20140227344 and EP2694529, which are incorporated herein by reference in their entirety, for exemplary VEPEP-6 peptides.

In some embodiments, the VEPEP-6 peptide comprises an amino acid sequence selected from the group consisting of LX₁RALWX₈LX₂X₈X₃LWX₈LX₄X₅X₆X₇(SEQ ID NO: 80), LX₁LARWX₈LX₂X₈X₃LWX₈LX₄X₅X₆X₇(SEQ ID NO: 81), LX₁ARLWX₈LX₂X₈X₃LWX₈LX₄X₅X₆X₇(SEQ ID NO: 82), LX₁RALWRLX₂RX₃LWRLX₄X₅X₆X₇(SEQ ID NO: 83), wherein X₁ is F or W, X₂ is L, W, C or I, X₃ is S, A, N or T, X₄ is L or W, X₅ is W or R, X₆ is K or R, X₇ is A or none, and X₈ is R or S. In some embodiments, the VEPEP-6 peptide comprises an amino acid of LX₁RALWRLX₂RX₃LWRLX₄X₅KX₆ (SEQ ID NO: 84), wherein X₁ is F or W, X₂ is L or W, X₃ is S, A or N, X₄ is L or W, X₅ is W or R, X₆ is A or none. In some embodiments, the VEPEP-6 peptide comprises an amino acid set forth in any one of SEQ ID NOs: 85-90. In some embodiments, the VEPEP-6 peptide comprises an amino acid set forth in any one of SEQ ID NOs: 91-97. In some embodiments, the VEPEP-6 peptide comprises an amino acid of LWRALWRLWRSLWRLLWK (SEQ ID NO: 97).

In some embodiments, the VEPEP-6 peptide is a retro-inverso peptide. In some embodiments, the retro-inverso peptide comprises a sequence of SEQ ID NO: 98.

In some embodiments, the VEPEP-6 peptide comprises two residues separated by three or six residues that are linked by a hydrocarbon linkage. In some embodiments, the VEPEP-6 peptide comprises a hydrocarbon linkage between two residues at positions 7 and 11. In some embodiments, the VEPEP-6 peptide comprises a hydrocarbon linkage between two residues at positions 10 and 14. In some embodiments, the VEPEP-6 peptide comprises a hydrocarbon linkage between two residues at positions 4 and 11. In some embodiments, the VEPEP-6 peptide comprises the amino acid sequence set forth in any one of SEQ ID NOs: 99-107.

### VEPEP-9 Peptides

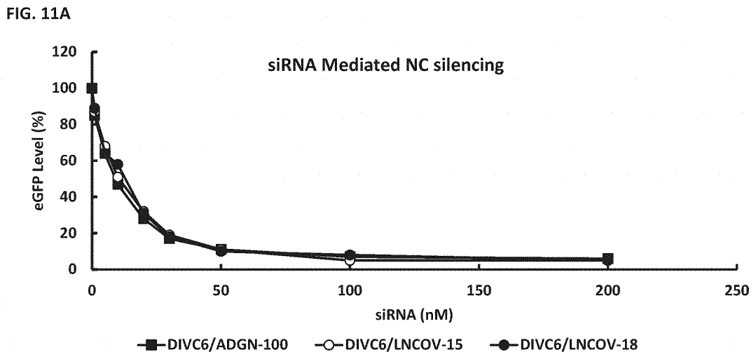

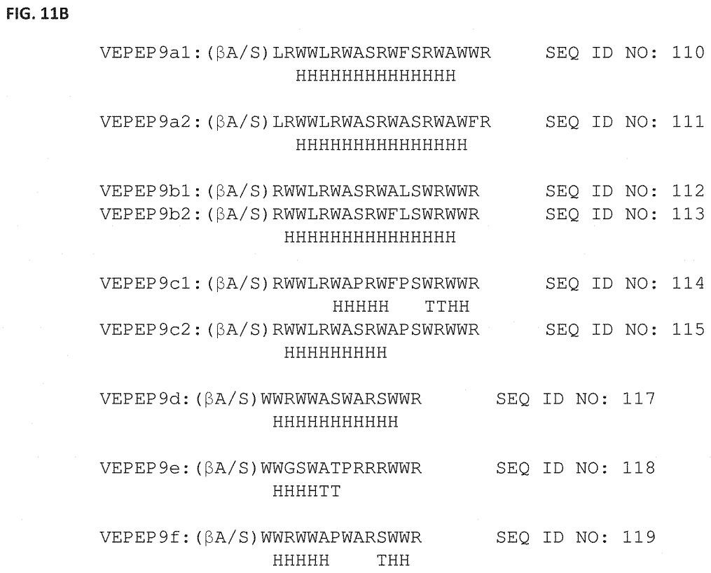

In some embodiments, the stabilizing peptide comprises a VEPEP-9 peptide (also referred to as a VEPEP-109 peptide). VEPEP-9 peptides, except VEPEP-9c and VEPEP-9e, are secondary amphipathic peptides; they are highly versatile and show a strong structural polymorphism. See FIG. 11B. VEPEP-9 are unfolded in solution as a free form and adopt an alpha helical conformation in the presence of lipid or artificial cellular membranes as well as in the presence of cargos such as peptide, SMH and small oligonucleotide. In contrast VEPEP-9c and VEPEP-9e adopt a coil/turn organization due to the presence of the proline residue in the sequence. The N-terminus domain of VEPEP-9c adopt an alpha helical conformation in the presence of lipid or artificial cellular membranes as well as in the presence of cargos. It is expected that VEPEP-9c and VEPEP-9e can also function as stabilizing peptides. See exemplary VEPEP-9 peptides described in WO2014/053624, US20160060296A1 and EP2928908, which are incorporated herein by reference in their entirety.

In some embodiments, the complex or nanoparticle described herein comprises a VEPEP-9 cell-penetrating peptide comprising the amino acid sequence X₁X₂X₃WWX₄X₅WAX₆X₃X₇X₈X₉X₁₀X₁₁X₁₂WX₁₃R (SEQ ID NO: 108), wherein X₁ is beta-A, S or none, X₂ is L or none, X₃ is R or none, X₄ is L, R or G, X₅ is R, W or S, X₆ is S, P or T, X₇ is W or P, X₈ is F, A or R, X₉ is S, L, P or R, X₁₀ is R or S, X₁₁ is W or none, X₁₂ is A, R or none and X₁₃ is W or F, and wherein if X₃ is none, then X₂, X₁₁ and X₁₂ are none as well. In some embodiments, the VEPEP-9 peptide comprises the amino acid sequence X₁X₂RWWLRWAX₆RWX₈X₉X₁₀WX₁₂WX₁₃R (SEQ ID NO: 109), wherein X₁ is beta-A, S or none, X₂ is L or none, X₆ is S or P, X₈ is F or A, X₉ is S, L or P, X₁₀ is R or S, X₁₂ is A or R, and X₁₃ is W or F. In some embodiments, the VEPEP-9 peptide comprises an amino acid sequence selected from the group consisting of X₁LRWWLRWASRWFSRWAWWR (SEQ ID NO: 110), X₁LRWWLRWASRWASRWAWFR (SEQ ID NO: 111), X₁RWWLRWASRWALSWRWWR (SEQ ID NO: 112), X₁RWWLRWASRWFLSWRWWR (SEQ ID NO: 113), X₁RWWLRWAPRWFPSWRWWR (SEQ ID NO: 114), and X₁RWWLRWASRWAPSWRWWR (SEQ ID NO: 115), wherein X₁ is beta-A, S or none. In some embodiments, the VEPEP-9 peptide comprises the amino acid sequence of X₁WWX₄X₅WAX₆X₇X₈RX₁₀WWR (SEQ ID NO: 116), wherein X₁ is beta-A, S or none, X₄ is R or G, X₅ is W or S, X₆ is S, T or P, X₇ is W or P, X₈ is A or R, and X₁₀ is S or R. In some embodiments, the VEPEP-9 peptide comprises an amino acid sequence selected from the group consisting of X₁WWRWWASWARSWWR (SEQ ID NO: 117), X₁WWGSWATPRRRWWR (SEQ ID NO: 118), and X₁WWRWWAPWARSWWR (SEQ ID NO: 119), wherein X₁ is beta-A, S, or none. In some embodiments, the VEPEP-9 peptide comprises the amino acid sequence set forth in SEQ ID NO: 120 or 121.

### VEPEP-3 Peptides

In some embodiments, the stabilizing peptide comprises a VEPEP-3 peptide. VEPEP-3 peptides are primary amphipathic peptides; they are highly versatile and show a strong structural polymorphism. VEPEP-3 peptides are unfolded in solution as a free form and adopt an alpha helical conformation in the N-terminal part in the presence of lipid or artificial cellular membranes as well as in the presence of cargos such as peptide or protein. Exemplary VEPEP-3 peptide are described, for example, in WO 2014/053622, US20160089447, and EP2951196, which are incorporated herein by reference in their entirety.

In some embodiments, the VEPEP-3 peptide comprises the amino acid sequence X₁X₂X₃X₄X₅X₂X₃X₄X₆X₇X₃X₈X₉X₁₀X₁X₁₂X₁₃ (SEQ ID NO: 122), wherein X₁ is beta-A, S or none, X₂ is K, R or L (independently from each other), X₃ is F or W (independently from each other), X₄ is F, W or Y (independently from each other), X₅ is E, R or S, X₆ is R, T or S, X₇ is E, R, or S, X₈ is none, F or W, X₉ is P or R, X₁₀ is R or L, X₁₁ is K, W or R, X₁₂ is R or F, and X₁₃ is R or K. In some embodiments, the VEPEP-3 peptide comprises the amino acid sequence X₁X₂WX₄EX₂WX₄X₆X₇X₃PRX₁₁RX₁₃ (SEQ ID NO: 123), wherein X₁ is beta-A, S or none, X₂ is K, R or L, X₃ is F or W, X₄ is F, W or Y, X₅ is E, R or S, X₆ is R, T or S, X₇ is E, R, or S, X₈ is none, F or W, X₉ is P or R, X₁₀ is R or L, X₁₁ is K, W or R, X₁₂ is R or F, and X₁₃ is R or K. In some embodiments, the VEPEP-3 peptide comprises the amino acid sequence X₁KWFERWFREWPRKRR (SEQ ID NO: 124), X₁KWWERWWREWPRKRR (SEQ ID NO: 125), X₁KWWERWWREWPRKRK (SEQ ID NO: 126), X₁RWWEKWWTRWPRKRK (SEQ ID NO: 127), or X₁RWYEKWYTEFPRRRR (SEQ ID NO: 128), wherein X₁ is beta-A, S or none. In some embodiments, the VEPEP-3 peptide comprises the amino acid sequence X₁KX₁₄WWERWWRX₁₄WPRKRK (SEQ ID NO: 129), wherein X₁ is beta-A, S or none, and X₁₄ is a non-natural amino acid, and wherein there is a hydrocarbon linkage between the two non-natural amino acids. In some embodiments, the VEPEP-3 peptide comprises the amino acid sequence X₁X₂X₃WX₅X₁₀X₃WX₆X₇WX₈X₉X₁₀WX₁₂R (SEQ ID NO: 130), wherein X₁ is beta-A, S or none, X₂ is K, R or L, X₃ is F or W, X₅ is R or S, X₆ is R or S, X₇ is R or S, X₈ is F or W, X₉ is R or P, X₁₀ is L or R, and X₁₂ is R or F. In some embodiments, the VEPEP-3 peptide comprises the amino acid sequence X₁RWWRLWWRSWFRLWRR (SEQ ID NO: 131), X₁LWWRRWWSRWWPRWRR (SEQ ID NO: 132), X₁LWWSRWWRSWFRLWFR (SEQ ID NO: 133), or X₁KFWSRFWRSWFRLWRR (SEQ ID NO: 134), wherein X₁ is beta-A, S or none. In some embodiments, the VEPEP-3 peptide comprises the amino acid sequence of any one of SEQ ID NOs: 122-134, wherein the cell-penetrating peptide is modified by replacement of the amino acids in position 5 and 12 by non-natural amino acids, and addition of a hydrocarbon linkage between the two non-natural amino acids. In some embodiments, the VEPEP-3 peptide comprises the amino acid sequence X₁RWWX₁₄LWWRSWX₁₄RLWRR (SEQ ID NO: 135), wherein X₁ is a beta-alanine, a serine, or none, and X₁₄ is a non-natural amino acid, and wherein there is a hydrocarbon linkage between the two non-natural amino acids. In some embodiments, the VEPEP-3 peptide comprises the amino acid sequence set forth in SEQ ID NO: 136 or 137.

### VEPEP-4 Peptides

In some embodiments, the stabilizing peptide comprises a VEPEP-4 peptide. VEPEP-4 peptides are amphipatic peptides; they are highly versatile and show a strong structural polymorphism. VEPEP-4 peptides are unfolded in solution in free form as well as in the presence of lipid or artificial cellular membranes or of cargos such as peptide or small molecules. Exemplary VEPEP-4 peptide are described, for example, in US20160115199A1, WO 2014/053628, and EP2928906, which are incorporated herein by reference in their entirety.

In some embodiments, the VEPEP-4 peptide comprises the amino acid sequence XWXRLXXXXXX (SEQ ID NO: 138), wherein X in position 1 is beta-A, S, or none; X in positions 3, 9 and 10 are, independently from each other, W or F; X in position 6 is R if X in position 8 is S, and X in position 6 is S if X in position 8 is R; X in position 7 is L or none; X in position 11 is R or none, and X in position 7 is L if X in position 11 is none. In some embodiments, the VEPEP-4 peptide comprises an amino acid sequence of any one of SEQ ID NOs: 138-142.

### VEPEP-5 Peptides

In some embodiments, the stabilizing peptide comprises a VEPEP-5 peptide. VEPEP-5 are short primary and, in certain cases, secondary amphipathic peptides forming stable nanoparticles with molecules such as peptides, peptide-analogues, PNAs and small hydrophobic molecules, hereafter designated as “SHM”. Exemplary VEPEP-5 peptides are described, for example, in US20160145299A1, WO 2014/053629, and EP2928907, which are incorporated herein by reference in their entirety.

In some embodiments, the VEPEP-5 peptide comprises the amino acid sequence RXWXRLWXRLR (SEQ ID NO: 143), wherein X in position 2 is R or S; and X in positions 4 and 8 are, independently from each other, W or F. In some embodiments, the VEPEP-5 peptide comprises an amino acid sequence of any one of SEQ ID NOs: 144-149.

### Linker

In some embodiments, the stabilizing peptide is connected to the blocking peptide via a linker.

In some embodiments, the linker comprises a polyglycine linker. In some embodiments, the linker comprises a β-Alanine. In some embodiments, the linker comprises at least about two, three, or four glycines, optionally continuous glycines. In some embodiments, the linker further comprises a serine. In some embodiments, the linker comprises a GGGGS (SEQ ID NO: 186) or SGGGG (SEQ ID NO: 187) sequence. In some embodiments, the linker comprises a Glycine-β-Alanine motif.

In some embodiments, the one or more moieties comprise a polymer (e.g., PEG, polylysine, PET). In some embodiments, the polymer is conjugated to the N-terminus of the CPP. In some embodiments, the polymer is conjugated to the C-terminus of the CPP. In some embodiments, a first polymer is conjugated to the N-terminus of the CPP and a second polymer is conjugated to the C-terminus of the CPP. In some embodiments, the polymer is a PEG. In some embodiments, the PEG is a linear PEG. In some embodiments, the PEG is a branched PEG. In some embodiments, the molecular weight of the PEG is no more than about 5 kDa, 10 kDa, 15 kDa, 20 kDa, 30 kDa, or 40 kDa. In some embodiments, the molecular weight of the PEG is at least about 5 kDa, 10 kDa, 15 kDa, 20 kDa, 30 kDa, or 40 kDa. In some embodiments, the molecular weight of the PEG is about 5 kDa to about 10 kDa, about 10 kDa to about 15 kDa, about 15 kDa to about 20 kDa, about 20 kDa to about 30 kDa, or about 30 kDa to about 40 kDa. In some embodiments, the molecular weight of the PEG is about 5 kDa, 10 kDa, 20 kDa, or 40 kDa. In some embodiments, the molecular weight of the PEG is selected from the group consisting of 5 kDa, 10 kDa, 20 kDa or 40 kDa. In some embodiments, the molecular weight of the PEG is about 5 kDa. In some embodiments, the molecular weight of the PEG is about 10 kDa. In some embodiments, the PEG comprises at least about 1, 2, or 3 ethylene glycol units. In some embodiments, the PEG consists of no more than about 10, 9, 8 or 7 ethylene glycol units. In some embodiments, the PEG consists of about 1, 2, or 3 ethylene glycol units. In some embodiments, the PEG moiety consists of about one to eight, or about two to about seven ethylene glycol units.

In some embodiments, the linker is selected from the group consisting of beta alanine, cysteine, cysteamide bridge, poly glycine (such as G2 or G4), Aun (11-amino-undecanoic acid), Ava (5-amino pentanoic acid), and Ahx (aminocaproic acid). In some embodiments, the linker moiety comprises Aun (11-amino-undecanoic acid). In some embodiments, the linker moiety comprises Ava (5-amino pentanoic acid). In some embodiments, the linker moiety comprises Ahx (aminocaproic acid).

In some embodiments, the linker comprises a proline. The proline creates a kick in the secondary structure between the blocking and stabilizing domain.

In some embodiments, the linker is selected from the group consisting of a proline, a polyglycine linker moiety, a PEG moiety (e.g., consisting of about 2-7 ethylene glycol units), Aun, Ava, and Ahx.

Nucleic acids encoding any of the chimeric peptides, blocking peptides or stabilizing peptides described herein are also contemplated.

siRNAs

The present application provides novel siRNAs that target SARS-CoV-2 virus.

In some embodiments, the siRNA targets SARS-CoV-2 nucleocapsid. In some embodiments, the siRNA comprises the nucleic acid sequence of any one of SEQ ID NOs: 161-170. In some embodiments, the siRNA comprises the nucleic acid sequence selected from the group consisting of SEQ ID NOs 163, 166, 168, and 170. In some embodiments, the siRNA comprises the nucleic acid sequence of SEQ ID NO: 166.

In some embodiments, the siRNA targets SARS-CoV-2 ORF 3A gene. In some embodiments, the siRNA comprises the nucleic acid sequence of any one of SEQ ID NOs: 171-175. In some embodiments, the siRNA comprises the nucleic acid sequence selected from the group consisting of SEQ ID NOs 171, 173, and 174.

In some embodiments, the siRNA targets SARS-CoV-2 ORF 8 gene. In some embodiments, the siRNA comprises the nucleic acid sequence of any one of SEQ ID NOs: 176-179. In some embodiments, the siRNA comprises the nucleic acid sequence selected from the group consisting of SEQ ID NOs 177, 178, and 180.

In some embodiments, the siRNA can be modified (e.g., chemically modified). Exemplary modifications include, but are not limited to 3′ terminal deoxy thymine, 2′ O methyl, a 2′ deoxy modification, a 2′ amino modification, a 2′ alkyl modification, a morpholino modification, a phosphoramidate modification, 5′-phosphorothioate group modification, a 5′ phosphate or 5′ phosphate mimic modification, a cholesteryl derivative or a dodecanoic acid bisdecylamide group modification. The modified nucleotide may be any one of a locked nucleotide, an abasic nucleotide or a non natural base comprising nucleotide.

Also provided herein are complexes, nanoparticles, compositions that comprise any of the chimeric peptides, blocking peptides, or siRNAs described above including, but not limited to the complexes, nanoparticles and compositions described as following.

Many delivery systems can be employed to deliver any of the chimeric peptides, blocking peptides, or siRNAs, complexes, nanoparticles, and compositions described in this application, including but not limited to, viral, liposome, electroporation, microinjection and conjugation, to achieve the introduction of the chimeric peptide(s), the blocking peptide(s) and/or siRNAs into a host cell. Conventional viral and non-viral based gene transfer methods can be used to introduce nucleic acids into mammalian cells or target tissues. Such methods can be used to administer nucleic acids encoding the chimeric or blocking peptides of the present invention to cells in culture, or in a host organism. Non-viral vector delivery systems include DNA plasmids, RNA (e.g. a transcript of a construct described herein), naked nucleic acid, and nucleic acid complexed with a delivery vehicle, such as a liposome.

Methods of non-viral delivery of nucleic acids include lipofection, nucleofection, microinjection, biolistics, virosomes, liposomes, immunoliposomes, polycation or lipid: nucleic acid conjugates, electroporation, nanoparticles, exosomes, microvesicles, or gene-gun, naked DNA and artificial virions.

The use of RNA or DNA viral based systems for the delivery of nucleic acids has high efficiency in targeting a virus to specific cells and/or trafficking the viral payload to the cellular nuclei.

### Complexes and Nanoparticles

The present application provides complexes comprising a) a cargo comprising a chimeric peptide, a blocking peptide, or a siRNA such as any of those described herein, and b) a second peptide, wherein the chimeric peptide, the blocking peptide, or siRNA is complexed with the second peptide. Also provided are nanoparticles comprising any of the complexes described herein. In some embodiments, there are provided nanoparticles comprising a) a cargo comprising a chimeric peptide, a blocking peptide, or a siRNA such as any of those described herein, and b) a second peptide.

In some embodiments, there is provided a complex or nanoparticle comprising a) a cargo comprising a chimeric peptide comprising a blocking peptide and a stabilizing peptide, wherein the blocking peptide specifically blocks interaction between SPIKE and ACE2 and comprises a loop sequence within the receptor-binding domain (RBD) of SPIKE, wherein the stabilizing peptide comprises an amphipathic helix, and b) a second peptide, wherein the second peptide is selected from the group consisting of CADY, MPG, PEP-1 peptides, PEP-2 peptides, PEP-3 peptides (e.g., SEQ ID NO: 183), LNCOV peptides, VEPEP-3 peptides, VEPEP-4 peptides, VEPEP-5 peptides, VEPEP-6 peptides, VEPEP-9 peptides, and ADGN-100 peptides. In some embodiments, the stabilizing peptide is selected from the group consisting of CADY, VEPEP-6 peptide, ADGN-100 peptide, and VEPEP-9 peptide. In some embodiments, the stabilizing peptide is connected to the C-terminus of the blocking peptide. In some embodiments, the loop sequence has a length of no more than about 20 amino acids (such as about 7-18 amino acids). In some embodiments, the blocking peptide comprises a lysine (K) at the C-terminus. In some embodiments, the loop sequence is cyclic. In some embodiments, the loop sequence is selected from the group consisting of SEQ ID NOs: 1-11 and 42-46. In some embodiments, the loop sequence is selected from the group consisting of SEQ ID NOs: 1, 6, 8-11, 42 and 45. In some embodiments, the blocking peptide and the stabilizing peptide are connected via a linker. In some embodiments, the linker is selected from the group consisting of a proline, a polyglycine linker moiety, a PEG moiety, Aun, Ava, and Ahx. In some embodiments, the second peptide is selected from the group consisting of an ADGN-100 peptide (e.g., SEQ ID NO: 55), VEPEP-6 peptide (e.g., SEQ ID NO: 97) or VEPEP-9 peptide (e.g., SEQ ID NO: 120 or 121). In some embodiments, the second peptide comprises an amino acid sequence selected from the group consisting of SEQ ID NOs: 53-79. In some embodiments, the second peptide comprises an amino acid sequence selected from the group consisting of SEQ ID NOs: 80-107. In some embodiments, the second peptide comprises an amino acid sequence selected from the group consisting of SEQ ID NOs: 108-121. In some embodiments, the molar ratio of the second peptide to the chimeric peptide is between about 1:1 to about 80:1 (e.g., about 2:1 to about 10:1, about 5:1). In some embodiments, the second peptide is complexed with the chimeric peptide.

In some embodiments, there is provided a complex or nanoparticle comprising a) a cargo comprising a chimeric peptide comprising a blocking peptide and a stabilizing peptide, wherein the blocking peptide specifically blocks interaction between SPIKE and ACE2 and comprises a sequence derived from a sequence within the extracellular domain of ACE2, wherein the stabilizing peptide comprises an amphipathic helix, and b) a second peptide, wherein the second peptide is selected from the group consisting of CADY, MPG, PEP-1 peptides, PEP-2 peptides, PEP-3 peptides (e.g., SEQ ID NO: 183), LNCOV peptides, VEPEP-3 peptides, VEPEP-4 peptides, VEPEP-5 peptides, VEPEP-6 peptides, VEPEP-9 peptides, and ADGN-100 peptides. In some embodiments, the stabilizing peptide is selected from the group consisting of CADY, VEPEP-106 peptide, ADGN-100 peptide, and VEPEP-109 peptide. In some embodiments, the stabilizing peptide is connected to the C-terminus of the blocking peptide. In some embodiments, the blocking peptide comprises a sequence selected from the group consisting of SEQ ID NOs: 23-31 and 47-52. In some embodiments, the blocking peptide comprises a sequence selected from the group consisting of SEQ ID NOs: 23, 24, 26-28, 31 and 47-52. In some embodiments, the blocking peptide and the stabilizing peptide are connected via a linker. In some embodiments, the linker is selected from the group consisting of a proline, a polyglycine linker moiety, a PEG moiety, Aun, Ava, and Ahx. In some embodiments, the second peptide is selected from the group consisting of an ADGN-100 peptide (e.g., SEQ ID NO: 55), VEPEP-6 peptide (e.g., SEQ ID NO: 97) or VEPEP-9 peptide (e.g., SEQ ID NO: 120 or 121). In some embodiments, the second peptide comprises an amino acid sequence selected from the group consisting of SEQ ID NOs: 53-79. In some embodiments, the second peptide comprises an amino acid sequence selected from the group consisting of SEQ ID NOs: 80-107. In some embodiments, the second peptide comprises an amino acid sequence selected from the group consisting of SEQ ID NOs: 108-121. In some embodiments, the molar ratio of the second peptide to the chimeric peptide is between about 1:1 to about 80:1 (e.g., about 2:1 to about 10:1, about 5:1). In some embodiments, the second peptide is complexed with the chimeric peptide.

In some embodiments, there is provided a complex or nanoparticle comprising a) a cargo comprising a chimeric peptide comprising a blocking peptide connected to a stabilizing peptide, wherein the blocking peptide comprises a sequence selected from the group consisting of SEQ ID NOs: 1, 6, 8-11, 23, 24, 26-28, 3142, 45 and 47-52, wherein the stabilizing peptide comprises an ADGN-100 peptide or a VEPEP-6 peptide connected to the C-terminus of the blocking peptide via a linker, and b) a second peptide, wherein the second peptide is selected from the group consisting of CADY, MPG, PEP-1 peptides, PEP-2 peptides, PEP-3 peptides (e.g., SEQ ID NO: 183), LNCOV peptides, VEPEP-3 peptides, VEPEP-4 peptides, VEPEP-5 peptides, VEPEP-6 peptides, VEPEP-9 peptides, and ADGN-100 peptides. In some embodiments, the stabilizing peptide is selected from the group consisting of CADY, VEPEP-106 peptide, ADGN-100 peptide, and VEPEP-109 peptide. In some embodiments, the linker is selected from the group consisting of a proline, a polyglycine linker moiety, a PEG moiety, Aun, Ava, and Ahx. In some embodiments, the second peptide is selected from the group consisting of an ADGN-100 peptide (e.g., SEQ ID NO: 55), VEPEP-6 peptide (e.g., SEQ ID NO: 97) or VEPEP-9 peptide (e.g., SEQ ID NO: 120 or 121). In some embodiments, the second peptide comprises an amino acid sequence selected from the group consisting of SEQ ID NOs: 53-79. In some embodiments, the second peptide comprises an amino acid sequence selected from the group consisting of SEQ ID NOs: 80-107. In some embodiments, the second peptide comprises an amino acid sequence selected from the group consisting of SEQ ID NOs: 108-121. In some embodiments, the molar ratio of the second peptide to the chimeric peptide is between about 1:1 to about 80:1 (e.g., about 2:1 to about 10:1, about 5:1). In some embodiments, the second peptide is complexed with the chimeric peptide.

In some embodiments, there is provided a complex or nanoparticle comprising a) a cargo comprising a chimeric peptide comprising a blocking peptide connected to a stabilizing peptide (e.g., connected to C-terminus of the blocking peptide) via a linker, wherein the blocking peptide comprises a sequence selected from the group consisting of SEQ ID NOs: 1, 6, and 8-11, wherein the stabilizing peptide comprises the amino acid sequence of SEQ ID NO: 55 or 97, and b) a second peptide, wherein the second peptide is selected from the group consisting of CADY, MPG, PEP-1 peptides, PEP-2 peptides, PEP-3 peptides (e.g., SEQ ID NO: 183), LNCOV peptides, VEPEP-3 peptides, VEPEP-4 peptides, VEPEP-5 peptides, VEPEP-6 peptides, VEPEP-9 peptides, and ADGN-100 peptides. In some embodiments, the stabilizing peptide is selected from the group consisting of CADY, VEPEP-106 peptide, ADGN-100 peptide, and VEPEP-109 peptide. In some embodiments, the linker is selected from the group consisting of a proline, a polyglycine linker moiety, a PEG moiety, Aun, Ava, and Ahx. In some embodiments, the second peptide is selected from the group consisting of an ADGN-100 peptide (e.g., SEQ ID NO: 55), VEPEP-6 peptide (e.g., SEQ ID NO: 97) or VEPEP-9 peptide (e.g., SEQ ID NO: 120 or 121). In some embodiments, the second peptide comprises an amino acid sequence selected from the group consisting of SEQ ID NOs: 53-79. In some embodiments, the second peptide comprises an amino acid sequence selected from the group consisting of SEQ ID NOs: 80-107. In some embodiments, the second peptide comprises an amino acid sequence selected from the group consisting of SEQ ID NOs: 108-121. In some embodiments, the molar ratio of the second peptide to the chimeric peptide is between about 1:1 to about 80:1 (e.g., about 2:1 to about 10:1, about 5:1). In some embodiments, the second peptide is complexed with the chimeric peptide.

In some embodiments, there is provided a complex comprising a) a cargo comprising a chimeric peptide comprising a blocking peptide connected to a stabilizing peptide (e.g., connected to C-terminus of the blocking peptide) via a linker, wherein the blocking peptide comprises a cyclic peptide selected from the group consisting of SEQ ID NOs: 151, 156, and 158-160, wherein the stabilizing peptide comprises the amino acid sequence of SEQ ID NO: 55 or 97, and b) a second peptide, wherein the second peptide is selected from the group consisting of CADY, MPG, PEP-1 peptides, PEP-2 peptides, PEP-3 peptides (e.g., SEQ ID NO: 183), LNCOV peptides, VEPEP-3 peptides, VEPEP-4 peptides, VEPEP-5 peptides, VEPEP-6 peptides, VEPEP-9 peptides, and ADGN-100 peptides. In some embodiments, the stabilizing peptide is selected from the group consisting of CADY, VEPEP-106 peptide, ADGN-100 peptide, and VEPEP-109 peptide. In some embodiments, the linker is selected from the group consisting of a proline, a polyglycine linker moiety, a PEG moiety, Aun, Ava, and Ahx. In some embodiments, the second peptide is selected from the group consisting of an ADGN-100 peptide (e.g., SEQ ID NO: 55), VEPEP-6 peptide (e.g., SEQ ID NO: 97) or VEPEP-9 peptide (e.g., SEQ ID NO: 120 or 121). In some embodiments, the second peptide comprises an amino acid sequence selected from the group consisting of SEQ ID NOs: 53-79. In some embodiments, the second peptide comprises an amino acid sequence selected from the group consisting of SEQ ID NOs: 80-107. In some embodiments, the second peptide comprises an amino acid sequence selected from the group consisting of SEQ ID NOs: 108-121. In some embodiments, the molar ratio of the second peptide to the chimeric peptide is between about 1:1 to about 80:1 (e.g., about 2:1 to about 10:1, about 5:1). In some embodiments, the second peptide is complexed with the chimeric peptide.

In some embodiments, there is provided a complex or nanoparticle comprising a) a cargo comprising a chimeric peptide comprising a blocking peptide connected to a stabilizing peptide (e.g., connected to C-terminus of the blocking peptide) via a linker, wherein the blocking peptide comprises a sequence selected from the group consisting of SEQ ID NOs: 23, 24, 26-28, 31 and 47-52, wherein the stabilizing peptide comprises the amino acid sequence of SEQ ID NO: 55 or 97, and b) a second peptide, wherein the second peptide is selected from the group consisting of CADY, MPG, PEP-1 peptides, PEP-2 peptides, PEP-3 peptides (e.g., SEQ ID NO: 183), LNCOV peptides, VEPEP-3 peptides, VEPEP-4 peptides, VEPEP-5 peptides, VEPEP-6 peptides, VEPEP-9 peptides, and ADGN-100 peptides. In some embodiments, the stabilizing peptide is selected from the group consisting of CADY, VEPEP-106 peptide, ADGN-100 peptide, and VEPEP-109 peptide. In some embodiments, the linker is selected from the group consisting of a proline, a polyglycine linker moiety, a PEG moiety, Aun, Ava, and Ahx. In some embodiments, the second peptide is selected from the group consisting of an ADGN-100 peptide (e.g., SEQ ID NO: 55), VEPEP-6 peptide (e.g., SEQ ID NO: 97) or VEPEP-9 peptide (e.g., SEQ ID NO: 120 or 121). In some embodiments, the second peptide comprises an amino acid sequence selected from the group consisting of SEQ ID NOs: 53-79. In some embodiments, the second peptide comprises an amino acid sequence selected from the group consisting of SEQ ID NOs: 80-107. In some embodiments, the second peptide comprises an amino acid sequence selected from the group consisting of SEQ ID NOs: 108-121. In some embodiments, the molar ratio of the second peptide to the chimeric peptide is between about 1:1 to about 80:1 (e.g., about 2:1 to about 10:1, about 5:1). In some embodiments, the second peptide is complexed with the chimeric peptide.

In some embodiments, there is provided a complex or nanoparticle comprising a) a cargo comprising a chimeric peptide comprising the amino acid sequence set forth in SEQ ID NOs: 17, 20, 21, 27, 28, 33, 39, and 40, and b) a second peptide, wherein the second peptide is selected from the group consisting of CADY, MPG, PEP-1 peptides, PEP-2 peptides, PEP-3 peptides (e.g., SEQ ID NO: 183), LNCOV peptides, VEPEP-3 peptides, VEPEP-4 peptides, VEPEP-5 peptides, VEPEP-6 peptides, VEPEP-9 peptides, and ADGN-100 peptides. In some embodiments, the stabilizing peptide is selected from the group consisting of CADY, VEPEP-106 peptide, ADGN-100 peptide, and VEPEP-109 peptide. In some embodiments, the linker is selected from the group consisting of a proline, a polyglycine linker moiety, a PEG moiety, Aun, Ava, and Ahx. In some embodiments, the second peptide is selected from the group consisting of an ADGN-100 peptide (e.g., SEQ ID NO: 55), VEPEP-6 peptide (e.g., SEQ ID NO: 97) or VEPEP-9 peptide (e.g., SEQ ID NO: 120 or 121). In some embodiments, the second peptide comprises an amino acid sequence selected from the group consisting of SEQ ID NOs: 53-79. In some embodiments, the second peptide comprises an amino acid sequence selected from the group consisting of SEQ ID NOs: 80-107. In some embodiments, the second peptide comprises an amino acid sequence selected from the group consisting of SEQ ID NOs: 108-121. In some embodiments, the molar ratio of the second peptide to the chimeric peptide is between about 1:1 to about 80:1 (e.g., about 2:1 to about 10:1, about 5:1). In some embodiments, the second peptide is complexed with the chimeric peptide.

In some embodiments, there is provided a complex comprising a) a cargo comprising a blocking peptide comprising the amino acid sequence of any one of SEQ ID NOs: 27, 38, 39, 40, and b) a second peptide, wherein the second peptide is selected from the group consisting of CADY, MPG, PEP-1 peptides, PEP-2 peptides, PEP-3 peptides (e.g., SEQ ID NO: 183), LNCOV peptides, VEPEP-3 peptides, VEPEP-4 peptides, VEPEP-5 peptides, VEPEP-6 peptides, VEPEP-9 peptides, and ADGN-100 peptides. In some embodiments, the second peptide is selected from the group consisting of an ADGN-100 peptide (e.g., SEQ ID NO: 55), VEPEP-6 peptide (e.g., SEQ ID NO: 97) or VEPEP-9 peptide (e.g., SEQ ID NO: 120 or 121). In some embodiments, the second peptide comprises an amino acid sequence selected from the group consisting of SEQ ID NOs: 53-79. In some embodiments, the second peptide comprises an amino acid sequence selected from the group consisting of SEQ ID NOs: 80-107. In some embodiments, the second peptide comprises an amino acid sequence selected from the group consisting of SEQ ID NOs: 108-121. In some embodiments, the molar ratio of the second peptide to the blocking peptide is between about 1:1 to about 80:1 (e.g., about 2:1 to about 10:1, about 5:1). In some embodiments, the second peptide is complexed with the blocking peptide.

In some embodiments, there is provided a complex or nanoparticle comprising a) a cargo comprising a blocking peptide comprising the amino acid sequence of any of SEQ ID NOs: 12-22, 24-41, and 151-160, and b) a second peptide, wherein the second peptide is selected from the group consisting of CADY, MPG, PEP-1 peptides, PEP-2 peptides, PEP-3 peptides (e.g., SEQ ID NO: 183), LNCOV peptides, VEPEP-3 peptides, VEPEP-4 peptides, VEPEP-5 peptides, VEPEP-6 peptides, VEPEP-9 peptides, and ADGN-100 peptides. In some embodiments, the second peptide is selected from the group consisting of an ADGN-100 peptide (e.g., SEQ ID NO: 55), VEPEP-6 peptide (e.g., SEQ ID NO: 97) or VEPEP-9 peptide (e.g., SEQ ID NO: 120 or 121). In some embodiments, the second peptide comprises an amino acid sequence selected from the group consisting of SEQ ID NOs: 53-79. In some embodiments, the second peptide comprises an amino acid sequence selected from the group consisting of SEQ ID NOs: 80-107. In some embodiments, the second peptide comprises an amino acid sequence selected from the group consisting of SEQ ID NOs: 108-121. In some embodiments, the molar ratio of the second peptide to the blocking peptide is between about 1:1 to about 80:1 (e.g., about 2:1 to about 10:1, about 5:1). In some embodiments, the second peptide is complexed with the blocking peptide.

In some embodiments, there is provided a complex or nanoparticle comprising a) a cargo comprising a blocking peptide comprising the amino acid sequence of any of SEQ ID NOs: 12, 17, 19-22, 24, 26-28, 31-33, 35, 38-40, 151, 156, and 158-160, and b) a second peptide, wherein the second peptide is selected from the group consisting of CADY, MPG, PEP-1 peptides, PEP-2 peptides, PEP-3 peptides (e.g., SEQ ID NO: 183), LNCOV peptides, VEPEP-3 peptides, VEPEP-4 peptides, VEPEP-5 peptides, VEPEP-6 peptides, VEPEP-9 peptides, and ADGN-100 peptides. In some embodiments, the second peptide is selected from the group consisting of an ADGN-100 peptide (e.g., SEQ ID NO: 55), VEPEP-6 peptide (e.g., SEQ ID NO: 97) or VEPEP-9 peptide (e.g., SEQ ID NO: 120 or 121). In some embodiments, the second peptide comprises an amino acid sequence selected from the group consisting of SEQ ID NOs: 53-79. In some embodiments, the second peptide comprises an amino acid sequence selected from the group consisting of SEQ ID NOs: 80-107. In some embodiments, the second peptide comprises an amino acid sequence selected from the group consisting of SEQ ID NOs: 108-121. In some embodiments, the molar ratio of the second peptide to the blocking peptide is between about 1:1 to about 80:1 (e.g., about 2:1 to about 10:1, about 5:1). In some embodiments, the second peptide is complexed with the blocking peptide.

In some embodiments, there is provided a complex or nanoparticle comprising a) a cargo comprising a siRNA targets SARS-CoV-2 nucleocapsid, and b) a second peptide, wherein the second peptide is selected from the group consisting of CADY, MPG, PEP-1 peptides, PEP-2 peptides, PEP-3 peptides (e.g., SEQ ID NO: 183), LNCOV peptides, VEPEP-3 peptides, VEPEP-4 peptides, VEPEP-5 peptides, VEPEP-6 peptides, VEPEP-9 peptides, and ADGN-100 peptides. In some embodiments, the second peptide is selected from the group consisting of an ADGN-100 peptide (e.g., SEQ ID NO: 55), VEPEP-6 peptide (e.g., SEQ ID NO: 97) or VEPEP-9 peptide (e.g., SEQ ID NO: 120 or 121). In some embodiments, the second peptide comprises an amino acid sequence selected from the group consisting of SEQ ID NOs: 53-79. In some embodiments, the second peptide comprises an amino acid sequence selected from the group consisting of SEQ ID NOs: 80-107. In some embodiments, the second peptide comprises an amino acid sequence selected from the group consisting of SEQ ID NOs: 108-121. In some embodiments, the molar ratio of the second peptide to the siRNA is between about 1:1 to about 80:1 (e.g., about 5:1 to about 50:1, about 20:1). In some embodiments, the second peptide is complexed with the blocking peptide. In some embodiments, the siRNA comprises the nucleic acid sequence of any one of SEQ ID NOs: 161-170. In some embodiments, the siRNA comprises the nucleic acid sequence selected from the group consisting of SEQ ID NOs 163, 166, 168, and 170. In some embodiments, the siRNA comprises the nucleic acid sequence of SEQ ID NO: 166.

In some embodiments, there is provided a complex or nanoparticle comprising a) a cargo comprising a siRNA comprising the nucleic acid sequence selected from the group consisting of SEQ ID NOs 163, 166, 168, and 170 (e.g., SEQ ID NO: 66), and b) a second peptide selected from the group consisting of an ADGN-100 peptide (e.g., SEQ ID NO: 55), VEPEP-6 peptide (e.g., SEQ ID NO: 97) or VEPEP-9 peptide (e.g., SEQ ID NO: 120 or 121), wherein the siRNA is complexed with the second peptide. In some embodiments, the molar ratio of the second peptide to the siRNA is between about 1:1 to about 80:1 (e.g., about 5:1 to about 50:1, about 20:1).

In some embodiments, there is provided a complex or nanoparticle comprising a) a cargo comprising i) a chimeric peptide comprising the amino acid sequence set forth in SEQ ID NOs: 17, 20, 21, 27, 28, 33, 39, and 40, ii) a siRNA comprising the nucleic acid sequence selected from the group consisting of SEQ ID NOs 163, 166, 168, and 170 (e.g., SEQ ID NO: 166), and b) a second peptide, wherein the second peptide is selected from the group consisting of CADY, MPG, PEP-1 peptides, PEP-2 peptides, PEP-3 peptides (e.g., SEQ ID NO: 183), LNCOV peptides, VEPEP-3 peptides, VEPEP-4 peptides, VEPEP-5 peptides, VEPEP-6 peptides, VEPEP-9 peptides, and ADGN-100 peptides. In some embodiments, the second peptide is selected from the group consisting of an ADGN-100 peptide (e.g., SEQ ID NO: 55), VEPEP-6 peptide (e.g., SEQ ID NO: 97) or VEPEP-9 peptide (e.g., SEQ ID NO: 120 or 121). In some embodiments, the second peptide comprises an amino acid sequence selected from the group consisting of SEQ ID NOs: 53-79. In some embodiments, the second peptide comprises an amino acid sequence selected from the group consisting of SEQ ID NOs: 80-107. In some embodiments, the second peptide comprises an amino acid sequence selected from the group consisting of SEQ ID NOs: 108-121. In some embodiments, the molar ratio of the second peptide to the siRNA is between about 1:1 to about 80:1 (e.g., about 5:1 to about 50:1, about 20:1). In some embodiments, the molar ratio of the second peptide to the chimeric peptide is between about 1:1 to about 80:1 (e.g., about 2:1 to about 10:1, about 5:1).

In some embodiments, there is provided a complex or nanoparticle comprising a) a cargo comprising i) a blocking peptide comprising the amino acid sequence of any of SEQ ID NOs: 12, 17, 19-22, 24, 26-28, 31-33, 35, 38-40, 151, 156, and 158-160, ii) a siRNA comprising the nucleic acid sequence selected from the group consisting of SEQ ID NOs 163, 166, 168, and 170 (e.g., SEQ ID NO: 166), and b) a second peptide, wherein the second peptide is selected from the group consisting of CADY, MPG, PEP-1 peptides, PEP-2 peptides, PEP-3 peptides (e.g., SEQ ID NO: 183), LNCOV peptides, VEPEP-3 peptides, VEPEP-4 peptides, VEPEP-5 peptides, VEPEP-6 peptides, VEPEP-9 peptides, and ADGN-100 peptides. In some embodiments, the second peptide is selected from the group consisting of an ADGN-100 peptide (e.g., SEQ ID NO: 55), VEPEP-6 peptide (e.g., SEQ ID NO: 97) or VEPEP-9 peptide (e.g., SEQ ID NO: 120 or 121). In some embodiments, the second peptide comprises an amino acid sequence selected from the group consisting of SEQ ID NOs: 53-79. In some embodiments, the second peptide comprises an amino acid sequence selected from the group consisting of SEQ ID NOs: 80-107. In some embodiments, the second peptide comprises an amino acid sequence selected from the group consisting of SEQ ID NOs: 108-121. In some embodiments, the molar ratio of the second peptide to the siRNA is between about 1:1 to about 80:1 (e.g., about 5:1 to about 50:1, about 20:1). In some embodiments, the molar ratio of the second peptide to the blocking peptide is between about 1:1 to about 80:1 (e.g., about 2:1 to about 10:1, about 5:1).

### Cargo Molecule

In some embodiments, the cargo molecule of the complex or nanoparticle as described above is selected from the group consisting of a nucleic acid (such as any of the siRNAs described herein), a peptide (such as any of the chimeric peptides or blocking peptide described herein), and a peptide/nucleic complex.

### Nucleic Acid

In some embodiments, the cargo molecule of the complex or nanoparticle as described above comprises a nucleic acid. In some embodiments, the nucleic acid is selected from the group consisting of oligonucleotides, polynucleotides, single- or double-stranded oligo and polynucleotides, antisense oligonucleotides, various forms of RNAi, including for example siRNA, shRNA, etc., microRNA (miRNA), antagomirs, ribozymes, aptamers, plasmid DNA, etc. and suitable combinations of one or more thereof. In some embodiments, the nucleic acid is selected from the group consisting of a siRNA, an miRNA, a shRNA, a gRNA, an mRNA, a DNA, a DNA plasmid, an oligonucleotide and an analogue thereof. In some embodiments, the nucleic acid comprises an mRNA. In some embodiments, the nucleic acid comprises an RNAi. In some embodiments, the nucleic acid comprises an mRNA and an RNAi, and wherein the mRNA encodes a therapeutic protein for treating a disease or condition, and wherein the RNAi targets an RNA, wherein expression of the RNA is associated with SARS-CoV-2 or a disease or condition associated with SARS-CoV-2. In some embodiments, the molar ratio of the cell-penetrating peptide to the nucleic acid is between about 1:1 and about 100:1.

In some embodiments, the nucleic acids are single stranded oligonucleotides. In some embodiments, the nucleic acids are double stranded oligonucleotides. The nucleic acids described herein may be any of a range of length of up to, but not necessarily 200 nucleotides in the case of antisense oligonucleotides, RNAi, siRNA, shRNA, iRNA, antagomirs or up to 1000 kilo bases in the case of plasmid DNA.

In some embodiments, the nucleic acids are interference RNA, such as siRNA (such as any of the siRNAs described herein) or shRNA. The term “interfering RNA” or “RNAi” or “interfering RNA sequence” refers to single-stranded RNA (e.g., mature miRNA) or double-stranded RNA (i.e., duplex RNA such as siRNA, aiRNA, or pre-miRNA) that is capable of reducing or inhibiting the expression of a target gene or sequence (e.g., by mediating the degradation or inhibiting the translation of mRNAs which are complementary to the interfering RNA sequence) when the interfering RNA is in the same cell as the target gene or sequence, interfering RNA thus refers to the single-stranded RNA that is complementary to a target mRNA sequence or to the double-stranded RNA formed by two complementary strands or by a single, self-complementary strand. Interfering RNA may have substantial or complete identity to the target gene or sequence, or may comprise a region of mismatch (i.e., a mismatch motif). The sequence of the interfering RNA can correspond to the full-length target gene, or a subsequence thereof. Interfering RNA includes “small-interfering RNA” or “siRNA,” e.g., interfering RNA of about 15-60, 15-50, or 5-40 (duplex) nucleotides in length, more typically about 15-30, 15-25, or 19-25 (duplex) nucleotides in length, and is preferably about 20-24, 21-22, or 21-23 (duplex) nucleotides in length (e.g., each complementary sequence of the double-stranded siRNA is 15-60, 15-50, 15-40, 15-30, 15-25, or 19-25 nucleotides in length, preferably about 20-24, 21-22, or 21-23 nucleotides in length, and the double-stranded siRNA is about 15-60, 15-50, 15-40, 5-30, 5-25, or 19-25 base pairs in length, preferably about 8-22, 9-20, or 19-21 base pairs in length).

In some embodiments, the nucleic acids are double-stranded antisense RNA. Suitable length of the interference RNA are about 5 to about 200 nucleotides, or 10-50 nucleotides or base pairs or 15-30 nucleotides or base pairs n some embodiments, the interference RNA is substantially complementary (such as at least about 60%, 70%, 80%, 90%, 95%, 98%, 99%, or more identical) to the corresponding target gene. In some embodiments, the antisense RNA is modified, for example by incorporating non-naturally occurring nucleotides.

In some embodiments, the nucleic acid is an interfering RNA, such as a siRNA, that specifically targets an RNA molecule, such as an mRNA, encoding a protein involved in SARS-COV-2 or a disease associated with SARS-COV-2.

In some embodiments, the siRNA targets SARS-CoV-2 nucleocapsid. In some embodiments, the siRNA comprises the nucleic acid sequence of any one of SEQ ID NOs: 161-170. In some embodiments, the siRNA comprises the nucleic acid sequence selected from the group consisting of SEQ ID NOs 163, 166, 168, and 170. In some embodiments, the siRNA comprises the nucleic acid sequence of SEQ ID NO: 166.

In some embodiments, the siRNA targets SARS-CoV-2 ORF 3A gene. In some embodiments, the siRNA comprises the nucleic acid sequence of any one of SEQ ID NOs: 171-175. In some embodiments, the siRNA comprises the nucleic acid sequence selected from the group consisting of SEQ ID NOs 171, 173, and 174.

In some embodiments, the siRNA targets SARS-CoV-2 ORF 8 gene. In some embodiments, the siRNA comprises the nucleic acid sequence of any one of SEQ ID NOs: 176-179. In some embodiments, the siRNA comprises the nucleic acid sequence selected from the group consisting of SEQ ID NOs 177, 178, and 180.

In some embodiments, the nucleic acids are miRNA. A microRNA (abbreviated miRNA) is a short ribonucleic acid (RNA) molecule found in eukaryotic cells. A microRNA molecule has very few nucleotides (an average of 22) compared with other R As. miRNAs are post-transcriptional regulators that bind to complementary sequences on target messenger RNA transcripts (mRNAs), usually resulting in translational repression or target degradation and gene silencing. The human genome may encode over 1000 miRNAs, which may target about 60% of mammalian genes and are abundant in many human cell types. Suitable length of the miRNAs are about 5 to about 200 nucleotides, or 0-50 nucleotides or base pairs or 15-30 nucleotides or base pairs. In some embodiments, the miRNA s substantially complementary (such as at least about 60%, 70%, 80%, 90%, 95%, 98%, 99%, or more identical to) the corresponding target gene. In some embodiments, the antisense RNA is modified, for example by incorporating non-naturally occurring nucleotides.

In some embodiments, the nucleic acids are plasmid DNA or DNA fragments (for example DNA fragments of lengths of up to about 1000 bp). In addition, the plasmid DNA or DNA fragments may be hypermethylated or hypomethylated. In some embodiments, the plasmid DNA or DNA fragments encode one or more genes, and may contain regulatory elements necessary for the expression of said one or more genes. In some embodiments, the plasmid DNA or DNA fragments may comprise one or more genes that encode a selectable marker, allowing for maintenance of the plasmid DNA or DNA fragment in an appropriate host cell.

### Peptide

In some embodiments, the cargo molecule comprises a blocking peptide that inhibits the interaction between ACE2 and SPIKE (such as any of the blocking peptide described herein). In some embodiments, the cargo molecule comprises a chimeric peptide such as any of the chimeric peptides described herein.

In some embodiments, the cargo molecule comprises a blocking peptide that specifically targets ACE2 (e.g., human ACE2). In some embodiments, the blocking peptide comprises an amino acid sequence derived from spike protein (e.g., human ACE2). In some embodiments, the amino acid sequence comprises a loop sequence within the receptor-binding domain (RBD) of SPIKE. In some embodiments, the loop sequence is within the receptor-binding motif (RBM) of SPIKE. In some embodiments, the loop sequence is within any of the RBM structure motif selected from the groups consisting of loop α4-β5, loop β5-β6, and loop β6-α5.

In some embodiments, the loop sequence is selected from the group consisting of SEQ ID NOs: 1-11 and 42-46. In some embodiments, the loop sequence is selected from the group consisting of SEQ ID NOs: 1, 6, 8-11, 42 and 45. In some embodiments, the blocking peptide comprises a sequence selected from the group consisting of SEQ ID NOs: 1, 6, and 8-11.

In some embodiments, the cargo molecule comprises an inhibitory peptide that specifically targets spike protein. In some embodiments, the inhibitory peptide comprises an amino acid sequence derived from ACE2 (e.g., human ACE2).

In some embodiments, the blocking peptide comprises a sequence derived from a sequence (such as a loop sequence, or an alpha helix motif) within the extracellular domain of ACE2 (such as human ACE2). In some embodiments, the blocking peptide comprises a sequence derived from a sequence within al helix of human ACE2. In some embodiments, the blocking peptide comprises a sequence derived from a sequence within α1 and α2 helices of human ACE2. In some embodiments, the blocking peptide comprises a sequence derived from a loop sequence between 33 and 34 strands of human ACE2.

In some embodiments, the sequence derived from a sequence within the extracellular domain of ACE2 is different from the sequence within the extracellular domain of ACE2 in one, two or three amino acids at the C-terminus of the sequence.

In some embodiments, the blocking peptide comprises a sequence within the extracellular domain of ACE2.

In some embodiments, the blocking peptide comprises a sequence selected from the group consisting of SEQ ID NOs: 23-31 and 47-52. In some embodiments, the blocking peptide comprises a sequence selected from the group consisting of SEQ ID NOs: 23, 24, 26-28, 31 and 47-52. In some embodiments, the blocking peptide comprises a sequence selected from the group consisting of SEQ ID NOs: 23, 24, 26-28 and 31.

In some embodiments, the blocking peptide comprises a cyclic peptide selected from any one of SEQ ID NOs: 151-160. In some embodiments, the blocking peptide comprises a cyclic peptide selected from the group consisting of SEQ ID NOs: 151, 156, and 158-160.

In some embodiments, the blocking peptide comprises a non-naturally occurring peptide selected from any one of SEQ ID NOs: 12-22 and 24-41.

### Peptide/Nucleic Complex

In some embodiments, the cargo comprises two or more cargo molecules comprising a) a nucleic acid (such as a siRNA that specifically targets an RNA molecule, such as an mRNA, encoding a protein involved in SARS-COV-2 or a disease associated with SARS-COV-2) and b) a blocking peptide that inhibits the interaction between ACE2 and SPIKE. In some embodiments, the nucleic acid is a siRNA that targets SARS-CoV-2 nucleocapsid.

In some embodiments, the molar ratio of the blocking peptide to the siRNA is between about 1:5 to about 50:1 (e.g., about 1:1 to about 20:1, about 2:1 to about 10:1, about 4:1).

Exemplary nucleic acids include any of the siRNAs described herein. In some embodiments, the nucleic acid comprises a siRNA comprising the nucleic acid sequence selected from the group consisting of SEQ ID NOs 163, 166, 168, and 170. In some embodiments, the siRNA comprises the nucleic acid sequence of SEQ ID NO: 166.

Exemplary blocking peptide include any of the chimeric peptides or blocking peptides described herein. In some embodiments, the blocking peptide comprises a) a cyclic peptide selected from the group consisting of SEQ ID NOs: 151, 156, and 158-160 or b) a peptide comprising a sequence selected from the group consisting of SEQ ID NOs: 1, 6, 8-11, 12, 17, 19-24, 26-28, 31-33, 35, 38-40, 45, 47, 48, 50, and 52.

### Second Peptide

In some embodiments, the second peptides described herein are able to self-assemble in nanostructure (e.g, has a diameter no more than 200 nm) after complexed with the cargo.

The second peptide can be any of the stabilizing peptides described above.

### Cell-Penetrating Peptides

In some embodiments, the second peptide is a cell penetrating peptide. Cell Penetrating Peptides (CPP) are one of the promising non-viral strategies. Although definition of CPPs is constantly evolving, they are generally described as short peptides of less than 30 amino acids either derived from proteins or from chimeric sequences. They are usually amphipathic and possess a net positive charge (Langel U (2007) Handbook of Cell-Penetrating Peptides (CRC Taylor & Francis, Boca Raton); Heitz et al. (2009) Br J Pharmacol 157, 195-206). CPPs are able to penetrate biological membranes, to trigger the movement of various biomolecules across cell membranes into the cytoplasm and to improve their intracellular routing, thereby facilitating interactions with the target. CPPs can be subdivided into two main classes, the first requiring chemical linkage with the cargo and the second involving the formation of stable, non-covalent complexes. CPPs from both strategies have been reported to favour the delivery of a large panel of cargos (plasmid DNA, oligonucleotide, siRNA, PNA, protein, peptide, liposome, nanoparticle . . . ) into a wide variety of cell types and in vivo models (Langel U (2007) Handbook of Cell-Penetrating Peptides (CRC Taylor & Francis, Boca Raton); Heitz et al. (2009) Br J Pharmacol 157, 195-206; Mickan et al. (2014) Curr Pharm Biotechnol 15, 200-209; Shukla et al. (2014) Mol Pharm 11, 3395-3408).

The concept of protein transduction domain (PTD) was initially proposed based on the observation that some proteins, mainly transcription factors, could shuttle within cells and from one cell to another (for review see Langel U (2007) Handbook of Cell-Penetrating Peptides (CRC Taylor & Francis, Boca Raton); Heitz et al. (2009) Br J Pharmacol 157, 195-206). The first observation was made in 1988, by Frankel and Pabo. They showed that the transcription-transactivating (Tat) protein of HIV-1 could enter cells and translocate into the nucleus. In 1991, the group of Prochiantz reached the same conclusions with the Drosophila Antennapedia homeodomain and demonstrated that this domain was internalized by neuronal cells. These works were at the origin of the discovery in 1994 of the first Protein Transduction Domain: a 16 mer-peptide derived from the third helix of the homeodomain of Antennapedia named Penetratin. In 1997, the group of Lebleu identified the minimal sequence of Tat required for cellular uptake, and the first proofs-of-concept of the application of PTD in vivo were reported by the group of Dowdy for the delivery of small peptides and large proteins (Gump J M, and Dowdy S F (2007) Trends Mol Med 13, 443-448). Historically, the notion of Cell Penetrating Peptide (CPP) was introduced by the group of Langel, in 1998, with the design of the first chimeric peptide carrier, the Transportan, which derived from the N-terminal fragment of the neuropeptide galanin, linked to mastoparan, a wasp venom peptide. Transportan has been originally reported to improve the delivery of PNAs (peptide nucleic acids) both in cultured cells and in vivo (Langel U (2007) Handbook of Cell-Penetrating Peptides (CRC Taylor & Francis, Boca Raton)). In 1997, the group of Heitz and Divita proposed a new strategy involving CPP in the formation of stable but non-covalent complexes with their cargo (Morris et al. (1997) Nucleic Acids Res 25, 2730-2736). The strategy was first based on the short peptide carrier (MPG) consisting of two domains: a hydrophilic (polar) domain and a hydrophobic (apolar) domain. MPG was designed for the delivery of nucleic acids. The primary amphipathic peptide Pep-1 was then proposed for non-covalent delivery of proteins and peptides (Morris et al. (2001) Nat Biotechnol 19, 1173-1176). Then the groups of Wender and of Futaki demonstrated that polyarginine sequences (Arg8) are sufficient to drive small and large molecules into cells and in vivo (Nakase et al. (2004) Mol Ther 10, 1011-1022; Rothbard et al. (2004) J Am Chem Soc 126, 9506-9507). Ever since, many CPPs derived from natural or unnatural sequences have been identified and the list is constantly increasing. Peptides have been derived from VP22 protein of Herpes Simplex Virus, from calcitonin, from antimicrobial or toxin peptides, from proteins involved in cell cycle regulation, as well as from polyproline-rich peptides (Heitz et al. (2009) Br J Pharmacol 157, 195-206). More recently, a new non-covalent strategy based on secondary amphipathic CPPs has been described. These peptides such as CADY and VEPEP-families described above are able to self-assemble in a helical shape with hydrophilic and hydrophobic residues on different side of the molecule (i.e., amphipathic helix). WO2014/053879 discloses VEPEP-3 peptides; WO2014/053881 discloses VEPEP-4 peptides; WO2014/053882 discloses VEPEP-5 peptides; WO2012/137150 discloses VEPEP-6 peptides; WO2014/053880 discloses VEPEP-9 peptides; WO 2016/102687 discloses ADGN-100 peptides; US2010/0099626 discloses CADY peptides; and. U.S. Pat. No. 7,514,530 discloses MPG peptides; the disclosures of which are hereby incorporated herein by reference in their entirety.

In some embodiments, the second peptide is selected from the group consisting of CADY, MPG, PEP-1 peptides (e.g., SEQ ID NO: 181), PEP-2 peptides (e.g., SEQ ID NO: 182), PEP-3 peptides (e.g., SEQ ID NO: 183), LNCOV peptides, VEPEP-3 peptides, VEPEP-6 peptides, VEPEP-9 peptides, and ADGN-100 peptides. In some embodiments, the second peptide comprises an ADGN-100 peptide. In some embodiments, the second peptide comprises a VEPEP-6 peptide. In some embodiments, the second peptide is a VEPEP-9 peptide.

### LNCOV Peptides

In some embodiments, the second peptide comprises a LNCOV peptide. As illustrated in Example 13, exemplary LNCOV peptide (LNCOV-15 and LNCOV-18) promotes cargo (such as siRNA) delivery in cells. In some embodiments, the LNCOV peptide comprises the amino acid sequence of any one of SEQ IDs NOs: 12-22, 27, 28, and 31-41. In some embodiments, the LNCOV peptide comprise the amino acid sequence of SEQ ID NO: 17 or 33.

### Second Peptide Modification

In some embodiments, the second peptide (such as CPP described herein, e.g., VEPEP-3 peptide, VEPEP-6 peptide, VEPEP-9 peptide, or ADGN-100 peptide) further comprises one or more moieties linked to (e.g., covalently linked to) the N-terminus of the second peptide. In some embodiments, the one or more moieties is covalently linked to the N-terminus of the second peptide. In some embodiments, the one or more moieties are selected from the group consisting of an acetyl group, a stearyl group, a fatty acid, a cholesterol, a poly-ethylene glycol, a nuclear localization signal, a nuclear export signal, an antibody or antibody fragment thereof, a peptide, a polysaccharide, a linker moiety, and a targeting moiety. In some embodiments, the one or more moieties comprise an acetyl group covalently linked to the N-terminus of the second peptide.

In some embodiments, the second peptide described herein (e.g., VEPEP-3 peptide, VEPEP-6 peptide, VEPEP-9 peptide, or ADGN-100 peptide) further comprises one or more moieties linked to (e.g., covalently linked to) the C-terminus of the second peptide. In some embodiments, the one or more moieties are selected from the group consisting of a cysteamide group, a cysteine, a thiol, an amide, a nitrilotriacetic acid, a carboxyl group, a linear or ramified C1-C6 alkyl group, a primary or secondary amine, an osidic derivative, a lipid, a phospholipid, a fatty acid, a cholesterol, a poly-ethylene glycol, a nuclear localization signal, a nuclear export signal, an antibody or antibody fragment thereof, a peptide, a polysaccharide, a linker moiety, and a targeting moiety. In some embodiments, the one or more moieties comprises a cysteamide group.

In some embodiments, the second peptide described herein (e.g., PEP-1, PEP-2, VEPEP-3 peptide, VEPEP-4 peptide, VEPEP-5 peptide, VEPEP-6 peptide, VEPEP-9 peptide, or ADGN-100 peptide) is stapled. “Stapled” as used herein refers to a chemical linkage between two residues in a peptide. In some embodiments, the second peptide is stapled, comprising a chemical linkage between two amino acids of the peptide. In some embodiments, the two amino acids linked by the chemical linkage are separated by 3 or 6 amino acids. In some embodiments, two amino acids linked by the chemical linkage are separated by 3 amino acids. In some embodiments, the two amino acids linked by the chemical linkage are separated by 6 amino acids. In some embodiments, each of the two amino acids linked by the chemical linkage is R or S. In some embodiments, each of the two amino acids linked by the chemical linkage is R. In some embodiments, each of the two amino acids linked by the chemical linkage is S. In some embodiments, one of the two amino acids linked by the chemical linkage is R and the other is S. In some embodiments, the chemical linkage is a hydrocarbon linkage.

In some embodiments, the second peptide is an L-peptide comprising L-amino acids. In some embodiments, the second peptide is a retro-inverso peptide (e.g., a peptide made up of D-amino acids in a reversed sequence and, when extended, assumes a side chain topology similar to that of its parent molecule but with inverted amide peptide bonds). In some embodiments, the retro-inverso peptide comprises a sequence of SEQ ID NO: 66, 67 or 98.

In some embodiments, the CPP comprises, from N-terminus, an acetyl group, a targeting moiety and a linker moiety covalently linked to the N-terminus of the cell-penetrating peptide.

### Targeting Moiety

In some embodiments, the second peptide comprise a targeting moiety. In some embodiments, the targeting moiety promotes the delivery of the complex or nanoparticles into a particular organ or tissue. In some embodiments, the targeting moiety is conjugated to the N-terminus the second peptide. In some embodiments, the targeting moiety is conjugated to the C-terminus the second peptide. In some embodiments, a first targeting moiety is conjugated to the N-terminus of the second peptide and a second targeting moiety is conjugated to the C-terminus of the second peptide.

In some embodiments, the targeting moiety comprises a targeting peptide that targets one or more organs. In some embodiments, the one or more organs are selected from the group consisting of muscle, heart, brain, spleen, lymph node, liver, lung, and kidney. In some embodiments, the targeting peptide targets lung. In some embodiments, the targeting peptide comprises an amino acid sequence of YIGSR (SEQ ID NO: 185).

In some embodiments, the targeting moiety is connected to the second peptide via a linker (such as any of the linkers described herein).

### Nanoparticles

The present application in one aspect provides a nanoparticle comprising a core comprising any one or more of complexes described above.

In some embodiments, there is provided a nanoparticle comprising a core comprising a complex described herein, wherein the second peptide (such as a cell-penetrating peptide) in the complex is associated with the cargo. In some embodiments, the association is non-covalent. In some embodiments, the association is covalent.

In some embodiments, the nanoparticle further comprises a surface layer (e.g., a shell) comprising a peripheral cell-penetrating peptide (i.e., CPP), wherein the core is coated by the shell. In some embodiments, the peripheral CPP is the same as the second peptide in the core. In some embodiments, the peripheral CPP is different than the second peptide in the core. In some embodiments, the peripheral CPP includes, but is not limited to, a PTD-based peptide, an amphipathic peptide, a poly-arginine-based peptide, an MPG peptide, a CADY peptide, a VEPEP peptide (such as a VEPEP-3, VEPEP-4, VEPEP-5, VEPEP-6, or VEPEP-9 peptide), an ADGN-100 peptide, a Pep-1 peptide, and a Pep-2 peptide. In some embodiments, the peripheral CPP is a VEPEP-3 peptide, a VEPEP-6 peptide, a VEPEP-9 peptide, or an ADGN-100 peptide. In some embodiments, the peripheral cell-penetrating peptide is selected from the group consisting of PEP-1 peptides, PEP-2 peptides, PEP-3 peptides, VEPEP-3 peptides, VEPEP-6 peptides, VEPEP-9 peptides, and ADGN-100 peptides. In some embodiments, at least some of the peripheral cell-penetrating peptides in the surface layer are linked to a targeting moiety. In some embodiments, the linkage is covalent. In some embodiments, the covalent linkage is by chemical coupling. In some embodiments, the covalent linkage is by genetic methods. In some embodiments, the nanoparticle further comprises an intermediate layer between the core of the nanoparticle and the surface layer. In some embodiments, the intermediate layer comprises an intermediate CPP. In some embodiments, the intermediate CPP is the same as the second peptide in the core. In some embodiments, the intermediate CPP is different than the second peptide in the core. In some embodiments, the intermediate CPP includes, but is not limited to, a PTD-based peptide, an amphipathic peptide, a poly-arginine-based peptide, an MPG peptide, a CADY peptide, a VEPEP peptide (such as a VEPEP-3, VEPEP-6, or VEPEP-9 peptide), an ADGN-100 peptide, a Pep-1 peptide, and a Pep-2 peptide. In some embodiments, the intermediate CPP is a VEPEP-3 peptide, a VEPEP-6 peptide, a VEPEP-9 peptide, or an ADGN-100 peptide.

In some embodiments, the nanoparticle core comprises a plurality of complexes. In some embodiments, the nanoparticle core comprises a plurality of complexes present in a predetermined ratio. In some embodiments, the predetermined ratio is selected to allow the most effective use of the nanoparticle in any of the methods described below in more detail. In some embodiments, the nanoparticle core further comprises one or more additional siRNA, one or more additional chimeric or blocking peptide described herein, and/or one or more second peptide (such as a cell-penetrating peptide).

In some embodiments, the nanoparticle comprises a) a first complex comprising a first chimeric peptide or blocking peptide (such as any of the chimeric peptide or blocking peptide described herein) complexed with a second peptide (such as any of the second peptide described herein), and b) a second complex comprising a second chimeric peptide or blocking peptide (such as any of the chimeric peptide or blocking peptide described herein) complexed with a third peptide (such as any of the peptides described in “the second peptide” section), wherein the first chimeric or blocking peptide targets ACE2, and wherein the second chimeric or blocking peptide targets SPIKE. In some embodiments, the first chimeric peptide or blocking peptide comprises a loop sequence selected from the group consisting of SEQ ID NOs: 1, 6, 8-11, 42 and 45. In some embodiments, the second chimeric peptide or blocking peptide comprises a sequence selected from the group consisting of SEQ ID NOs: 23, 24, 26-28, 31 and 47-52.

In some embodiments, the nanoparticle comprises a) a first complex comprising a chimeric peptide or blocking peptide (such as any of the chimeric peptide or blocking peptide described herein) complexed with a second peptide (such as any of the second peptide described herein), and b) a second complex comprising an siRNA (such as any of the siRNAs described herein) complexed with a third peptide (such as any of the peptides described in “the second peptide” section), wherein the chimeric or blocking peptide inhibits the interaction between ACE2 and SPIKE, and wherein the siRNA targets nucleocapsid of SPIKE. In some embodiments, the chimeric peptide or blocking peptide comprises the amino acid sequence set forth in SEQ ID NOs: 17, 20, 21, 27, 28, 33, 39, and 40. In some embodiments, the siRNA comprises the nucleic acid sequence selected from the group consisting of SEQ ID NOs 163, 166, 168, and 170 (e.g., SEQ ID NO: 166).

In some embodiments, the nanoparticle comprises a) a first complex comprising a first chimeric peptide or blocking peptide (such as any of the chimeric peptide or blocking peptide described herein) complexed with a second peptide (such as any of the second peptide described herein), b) a second complex comprising a second chimeric peptide or blocking peptide (such as any of the chimeric peptide or blocking peptide described herein) complexed with a third peptide (such as any of the peptides described in “the second peptide” section), and c) a third complex comprising an siRNA (such as any of the siRNAs described herein) complexed with a fourth peptide (such as any of the peptides described in “the second peptide” section), wherein the first chimeric or blocking peptide targets ACE2, wherein the second chimeric or blocking peptide targets SPIKE, and wherein the siRNA targets nucleocapsid of SPIKE. In some embodiments, the first chimeric peptide or blocking peptide comprises a loop sequence selected from the group consisting of SEQ ID NOs: 1, 6, 8-11, 42 and 45. In some embodiments, the second chimeric peptide or blocking peptide comprises a sequence selected from the group consisting of SEQ ID NOs: 23, 24, 26-28, 31 and 47-52. In some embodiments, the siRNA comprises the nucleic acid sequence selected from the group consisting of SEQ ID NOs 163, 166, 168, and 170 (e.g., SEQ ID NO: 166).

In some embodiments, the one or more of the second, third, and fourth peptides described above comprises a cell-penetrating peptide including, but not limited to, a PTD-based peptide, an amphipathic peptide, a poly-arginine-based peptide, an MPG peptide, a CADY peptide, a VEPEP peptide (such as a VEPEP-3, VEPEP-6, or VEPEP-9 peptide), an ADGN-100 peptide, a Pep-1 peptide, and a Pep-2 peptide. In some embodiments, at least some of the one or more of the second, third, and fourth peptides are linked to a targeting moiety. In some embodiments, the linkage is covalent.

In some embodiments, according to any of the nanoparticles described herein, the mean size (diameter) of the nanoparticle is from about 20 nm to about 1000 nm, including for example from about 20 nm to about 800 nm, from about 20 nm to about 600 nm, from about 20 nm to about 600 nm, and from about 20 nm to about 400 nm. In some embodiments, the mean size (diameter) of the nanoparticle is no greater than about 1000 nanometers (nm), such as no greater than about any of 900, 800, 700, 600, 500, 400, 300, 200,100, 90, 80, 70 or 60 nm. In some embodiments, the average or mean diameter of the nanoparticle is no greater than about 100 nm. In some embodiments, the average or mean diameters of the nanoparticles is no greater than about 60 nm. In some embodiments, the average or mean diameter of the nanoparticle is about 20 nm to about 100 nm. In some embodiments, the average or mean diameter of the nanoparticle is about 20 nm to about 80 nm. In some embodiments, the average or mean diameter of the nanoparticle is about 30 nm to about 60 nm.

In some embodiments, the average or mean diameters of the nanoparticles is no greater than about 150 nm. In some embodiments, the average or mean diameter of the nanoparticle is no greater than about 100 nm. In some embodiments, the average or mean diameter of the nanoparticle is about 20 nm to about 400 nm. In some embodiments, the average or mean diameter of the nanoparticle is about 30 nm to about 400 nm. In some embodiments, the average or mean diameter of the nanoparticle is about 40 nm to about 300 nm. In some embodiments, the average or mean diameter of the nanoparticle is about 50 nm to about 200 nm. In some embodiments, the average or mean diameter of the nanoparticle is about 60 nm to about 150 nm. In some embodiments, the average or mean diameter of the nanoparticle is about 70 nm to about 100 nm.

Methods of determining average particle sizes are known in the art, for example, dynamic light scattering (DLS) has been routinely used in determining the size of submicrometre-sized particles based. International Standard ISO22412 Particle Size Analysis—Dynamic Light Scattering, International Organisation for Standardisation (ISO) 2008 and Dynamic Light Scattering Common Terms Defined, Malvern Instruments Limited, 2011. In some embodiments, the particle size is measured as the volume-weighted mean particle size (Dv50) of the nanoparticles in the composition.

In some embodiments, the nanoparticles are sterile-filterable.

In some embodiments, the zeta potential of the nanoparticle is from about −30 mV to about 60 mV (such as about any of −30, −25, −20, −15, −10, −5, 0, 5, 10, 15, 20, 25, 30, 35, 40, 45, 50, 55, and 60 mV, including any ranges between these values). In some embodiments, the zeta potential of the nanoparticle is from about −30 mV to about 30 mV, including for example from about −25 mV to about 25 mV, from about −20 mV to about 20 mV, from about −15 mV to about 15 mV, from about −10 mV to about 10 mV, and from about −5 mV to about 10 mV. In some embodiments, the polydispersity index (PI) of the nanoparticle is from about 0.05 to about 0.6 (such as about any of 0.05, 0.1, 0.15, 0.2, 0.25, 0.3, 0.35, 0.4, 0.45, 0.5, 0.55, and 0.6, including any ranges between these values). In some embodiments, the polydispersity index (PI) of the nanoparticle is from about 0.1 to about 0.4, or about 0.15 to about 0.3. In some embodiments, the nanoparticle is substantially non-toxic. The zeta potential can be measured with Zetasizer 4 apparatus (Malvern Ltd).

### Compositions

In some embodiments, there is provided a composition (e.g., a pharmaceutical composition) comprising any of the chimeric peptides, blocking peptides, siRNAs, a complexes, or nanoparticles as described herein. In some embodiments, the composition is a pharmaceutical composition comprising any of the chimeric peptides, blocking peptides, siRNAs, a complexes, or nanoparticles as described herein and a pharmaceutically acceptable diluent, excipient, and/or carrier.

In some embodiments, the composition comprises a mixture of two or more nanoparticles, wherein the two or more nanoparticles comprise different cargos. For example, in some embodiments, the composition comprises a) a first nanoparticle as described above comprising a chimeric peptide or blocking peptide that specifically targets ACE2 or SPIKE, and b) a second nanoparticle comprising a siRNA that specifically targets nucleocapsid of SARS-CoV-2. In some embodiments, the chimeric peptide or blocking peptide comprises the amino acid sequence set forth in SEQ ID NOs: 17, 20, 21, 27, 28, 33, 39, and 40. In some embodiments, the siRNA comprises the nucleic acid sequence selected from the group consisting of SEQ ID NOs 163, 166, 168, and 170 (e.g., SEQ ID NO: 166).

In some embodiments, the concentration of the complex or nanoparticle in the composition is from about 1 nM to about 100 mM, including for example from about 10 nM to about 50 mM, from about 25 nM to about 25 mM, from about 50 nM to about 10 mM, from about 100 nM to about 1 mM, from about 500 nM to about 750 PM, from about 750 nM to about 500 μM, from about 1 μM to about 250 μM, from about 10 μM to about 200 PM, and from about 50 μM to about 150 μM. In some embodiments, the pharmaceutical composition is lyophilized.

The term “pharmaceutically acceptable diluent, excipient, and/or carrier” as used herein is intended to include any and all solvents, dispersion media, coatings, antibacterial and antifungal agents, isotonic and absorption delaying agents, and the like, compatible with administration to humans or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals. The term diluent, excipient, and/or “carrier” refers to a diluent, adjuvant, excipient, or vehicle with which the pharmaceutical composition is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like, including lyophilization aids. The composition, if desired, can also contain minor amounts of wetting, bulking, emulsifying agents, or pH buffering agents. These compositions can take the form of solutions, suspensions, emulsion, sustained release formulations and the like. Examples of suitable pharmaceutical diluent, excipient, and/or carriers are described in “Remington's Pharmaceutical Sciences” by E. W. Martin. The formulation should suit the mode of administration. The appropriate diluent, excipient, and/or carrier will be evident to those skilled in the art and will depend in large part upon the route of administration.

In some embodiments, a composition comprising a chimeric peptide, a blocking peptide, a siRNA, a complex or nanoparticle as described herein further comprises a pharmaceutically acceptable diluent, excipient, and/or carrier. In some embodiments, the pharmaceutically acceptable diluent, excipient, and/or carrier affects the level of aggregation of a complex or nanoparticle in the composition and/or the efficiency of intracellular delivery mediated by a complex or nanoparticle in the composition. In some embodiments, the extent and/or direction of the effect on aggregation and/or delivery efficiency mediated by the pharmaceutically acceptable diluent, excipient, and/or carrier is dependent on the relative amount of the pharmaceutically acceptable diluent, excipient, and/or carrier in the composition.

For example, in some embodiments, the presence of a pharmaceutically acceptable diluent, excipient, and/or carrier (such as a salt, sugar, chemical buffering agent, buffer solution, cell culture medium, or carrier protein) at one or more concentrations in the composition does not promote and/or contribute to aggregation of the complex or nanoparticle, or promotes and/or contributes to the formation of aggregates of the complex or nanoparticles having a size no more than about 200% (such as no more than about any of 190, 180, 170, 160, 150, 140, 130, 120, 110, 100, 90, 80, 70, 60, 50, 40, 30, 20, 10, 9, 8, 7, 6, 5, 4, 3, 2, or 1%, including any ranges between any of these values) larger than the size of the complex or nanoparticle. In some embodiments, the composition comprises the pharmaceutically acceptable diluent, excipient, and/or carrier at a concentration that does not promote and/or contribute to aggregation of the complex or nanoparticle, or promotes and/or contributes to the formation of aggregates of the complex or nanoparticles having a size no more than about 200% (such as no more than about any of 190, 180, 170, 160, 150, 140, 130, 120, 110, 100, 90, 80, 70, 60, 50, 40, 30, 20, 10, 9, 8, 7, 6, 5, 4, 3, 2, or 1%, including any ranges between any of these values) larger than the size of the complex or nanoparticle. In some embodiments, the composition comprises the pharmaceutically acceptable diluent, excipient, and/or carrier at a concentration that promotes and/or contributes to the formation of aggregates of the complex or nanoparticles having a size no more than about 150% larger than the size of the complex or nanoparticle. In some embodiments, the composition comprises the pharmaceutically acceptable diluent, excipient, and/or carrier at a concentration that promotes and/or contributes to the formation of aggregates of the complex or nanoparticles having a size no more than about 100% larger than the size of the complex or nanoparticle. In some embodiments, the composition comprises the pharmaceutically acceptable diluent, excipient, and/or carrier at a concentration that promotes and/or contributes to the formation of aggregates of the complex or nanoparticles having a size no more than about 50% larger than the size of the complex or nanoparticle. In some embodiments, the composition comprises the pharmaceutically acceptable diluent, excipient, and/or carrier at a concentration that promotes and/or contributes to the formation of aggregates of the complex or nanoparticles having a size no more than about 20% larger than the size of the complex or nanoparticle. In some embodiments, the composition comprises the pharmaceutically acceptable diluent, excipient, and/or carrier at a concentration that promotes and/or contributes to the formation of aggregates of the complex or nanoparticles having a size no more than about 15% larger than the size of the complex or nanoparticle. In some embodiments, the composition comprises the pharmaceutically acceptable diluent, excipient, and/or carrier at a concentration that promotes and/or contributes to the formation of aggregates of the complex or nanoparticles having a size no more than about 10% larger than the size of the complex or nanoparticle. In some embodiments, the pharmaceutically acceptable diluent, excipient, and/or carrier is a salt, including, without limitation, NaCl. In some embodiments, the pharmaceutically acceptable diluent, excipient, and/or carrier is a sugar, including, without limitation, sucrose, glucose, and mannitol. In some embodiments, the pharmaceutically acceptable diluent, excipient, and/or carrier is a chemical buffering agent, including, without limitation, HEPES. In some embodiments, the pharmaceutically acceptable diluent, excipient, and/or carrier is a buffer solution, including, without limitation, PBS. In some embodiments, the pharmaceutically acceptable diluent, excipient, and/or carrier is a cell culture medium, including, without limitation, DMEM. Particle size can be determined using any means known in the art for measuring particle size, such as by dynamic light scattering (DLS). For example, in some embodiments, an aggregate having a Z-average as measured by DLS that is 10% greater than the Z-average as measured by DLS of a complex or nanoparticle is 10% larger than the complex or nanoparticle.

In some embodiments, the composition comprises a salt (e.g., NaCl) at a concentration that does not promote and/or contribute to aggregation of the complex or nanoparticle, or promotes and/or contributes to the formation of aggregates of the complex or nanoparticles having a size no more than about 100% (such as no more than about any of 90, 80, 70, 60, 50, 40, 30, 20, 10, 9, 8, 7, 6, 5, 4, 3, 2, or 1%, including any ranges between any of these values) larger than the size of the complex or nanoparticle. In some embodiments, the composition comprises a salt (e.g., NaCl) at a concentration that promotes and/or contributes to the formation of aggregates of the complex or nanoparticles having a size no more than about 75% larger than the size of the complex or nanoparticle. In some embodiments, the composition comprises a salt (e.g., NaCl) at a concentration that promotes and/or contributes to the formation of aggregates of the complex or nanoparticles having a size no more than about 50% larger than the size of the complex or nanoparticle. In some embodiments, the composition comprises a salt (e.g., NaCl) at a concentration that promotes and/or contributes to the formation of aggregates of the complex or nanoparticles having a size no more than about 20% larger than the size of the complex or nanoparticle. In some embodiments, the composition comprises a salt (e.g., NaCl) at a concentration that promotes and/or contributes to the formation of aggregates of the complex or nanoparticles having a size no more than about 15% larger than the size of the complex or nanoparticle. In some embodiments, the composition comprises a salt (e.g., NaCl) at a concentration that promotes and/or contributes to the formation of aggregates of the complex or nanoparticles having a size no more than about 10% larger than the size of the complex or nanoparticle. In some embodiments, the concentration of the salt in the composition is no more than about 100 mM (such as no more than about any of 90, 80, 70, 60, 50, 40, 30, 20, 10, 9, 8, 7, 6, 5, 4, 3, 2, or 1 mM, including any ranges between any of these values). In some embodiments, the salt is NaCl.

In some embodiments, the composition comprises a sugar (e.g., sucrose, glucose, or mannitol) at a concentration that does not promote and/or contribute to aggregation of the complex or nanoparticle, or promotes and/or contributes to the formation of aggregates of the complex or nanoparticles having a size no more than about 25% (such as no more than about any of 24, 23, 22, 21, 20, 19, 18, 17, 16, 15, 14, 13, 12, 11, 10, 9, 8, 7, 6, 5, 4, 3, 2, or 1%, including any ranges between any of these values) larger than the size of the complex or nanoparticle. In some embodiments, the composition comprises a sugar (e.g., sucrose, glucose, or mannitol) at a concentration that promotes and/or contributes to the formation of aggregates of the complex or nanoparticles having a size no more than about 75% larger than the size of the complex or nanoparticle. In some embodiments, the composition comprises a sugar (e.g., sucrose, glucose, or mannitol) at a concentration that promotes and/or contributes to the formation of aggregates of the complex or nanoparticles having a size no more than about 50% larger than the size of the complex or nanoparticle. In some embodiments, the composition comprises a sugar (e.g., sucrose, glucose, or mannitol) at a concentration that promotes and/or contributes to the formation of aggregates of the complex or nanoparticles having a size no more than about 20% larger than the size of the complex or nanoparticle. In some embodiments, the composition comprises a sugar (e.g., sucrose, glucose, or mannitol) at a concentration that promotes and/or contributes to the formation of aggregates of the complex or nanoparticles having a size no more than about 15% larger than the size of the complex or nanoparticle. In some embodiments, the composition comprises a sugar (e.g., sucrose, glucose, or mannitol) at a concentration that promotes and/or contributes to the formation of aggregates of the complex or nanoparticles having a size no more than about 10% larger than the size of the complex or nanoparticle. In some embodiments, the concentration of the sugar in the composition is no more than about 20% (such as no more than about any of 18, 16, 14, 12, 10, 9, 8, 7, 6, 5, 4, 3, 2, or 1%, including any ranges between any of these values). In some embodiments, the sugar is sucrose. In some embodiments, the sugar is glucose. In some embodiments, the sugar is mannitol.

In some embodiments, the composition comprises a chemical buffering agent (e.g., HEPES or phosphate) at a concentration that does not promote and/or contribute to aggregation of the complex or nanoparticle, or promotes and/or contributes to the formation of aggregates of the complex or nanoparticles having a size no more than about 10% (such as no more than about any of 9, 8, 7, 6, 5, 4, 3, 2, or 1%, including any ranges between any of these values) larger than the size of the complex or nanoparticle. In some embodiments, the composition comprises a chemical buffering agent (e.g., HEPES or phosphate) at a concentration that promotes and/or contributes to the formation of aggregates of the complex or nanoparticles having a size no more than about 7.5% larger than the size of the complex or nanoparticle. In some embodiments, the composition comprises a chemical buffering agent (e.g., HEPES or phosphate) at a concentration that promotes and/or contributes to the formation of aggregates of the complex or nanoparticles having a size no more than about 5% larger than the size of the complex or nanoparticle. In some embodiments, the composition comprises a chemical buffering agent (e.g., HEPES or phosphate) at a concentration that promotes and/or contributes to the formation of aggregates of the complex or nanoparticles having a size no more than about 3% larger than the size of the complex or nanoparticle. In some embodiments, the composition comprises a chemical buffering agent (e.g., HEPES or phosphate) at a concentration that promotes and/or contributes to the formation of aggregates of the complex or nanoparticles having a size no more than about 1% larger than the size of the complex or nanoparticle. In some embodiments, the composition comprises a chemical buffering agent (e.g., HEPES or phosphate) at a concentration that does not promote and/or contribute to the formation of aggregates of the complex or nanoparticles. In some embodiments, the chemical buffering agent is HEPES. In some embodiments, the HEPES is added to the composition in the form of a buffer solution comprising HEPES. In some embodiments, the solution comprising HEPES has a pH between about 5 and about 9 (such as about any of 5, 5.5, 6, 6.5, 7, 7.5, 8, 8.5, and 9, including any ranges between these values). In some embodiments, the composition comprises HEPES at a concentration of no more than about 75 mM (such as no more than about any of 70, 65, 60, 55, 50, 45, 40, 35, 30, 25, 20, 15, 10 mM or less, including any ranges between any of these values). In some embodiments, the chemical buffering agent is phosphate. In some embodiments, the phosphate is added to the composition in the form of a buffer solution comprising phosphate. In some embodiments, the composition does not comprise PBS.

In some embodiments, the composition comprises a cell culture medium (e.g., DMEM or Opti-MEM) at a concentration that does not promote and/or contribute to aggregation of the complex or nanoparticle, or promotes and/or contributes to the formation of aggregates of the complex or nanoparticles having a size no more than about 200% (such as no more than about any of 190, 180, 170, 160, 150, 140, 130, 120, 110, 100, 90, 80, 70, 60, 50, 40, 30, 20, 10, 9, 8, 7, 6, 5, 4, 3, 2, or 1%, including any ranges between any of these values) larger than the size of the complex or nanoparticle. In some embodiments, the composition comprises a cell culture medium (e.g., DMEM or Opti-MEM) at a concentration that promotes and/or contributes to the formation of aggregates of the complex or nanoparticles having a size no more than about 150% larger than the size of the complex or nanoparticle. In some embodiments, the composition comprises a cell culture medium (e.g., DMEM or Opti-MEM) at a concentration that promotes and/or contributes to the formation of aggregates of the complex or nanoparticles having a size no more than about 100% larger than the size of the complex or nanoparticle. In some embodiments, the composition comprises a cell culture medium (e.g., DMEM or Opti-MEM) at a concentration that promotes and/or contributes to the formation of aggregates of the complex or nanoparticles having a size no more than about 50% larger than the size of the complex or nanoparticle. In some embodiments, the composition comprises a cell culture medium (e.g., DMEM or Opti-MEM) at a concentration that promotes and/or contributes to the formation of aggregates of the complex or nanoparticles having a size no more than about 25% larger than the size of the complex or nanoparticle. In some embodiments, the composition comprises a cell culture medium (e.g., DMEM or Opti-MEM) at a concentration that promotes and/or contributes to the formation of aggregates of the complex or nanoparticles having a size no more than about 10% larger than the size of the complex or nanoparticle. In some embodiments, the cell culture medium is DMEM. In some embodiments, the composition comprises DMEM at a concentration of no more than about 70% (such as no more than about any of 65, 60, 55, 50, 45, 40, 35, 30, 25, 20, 15, 10%, or less, including any ranges between any of these values).

In some embodiments, the composition comprises a carrier protein (e.g., albumin) at a concentration that does not promote and/or contribute to aggregation of the complex or nanoparticle, or promotes and/or contributes to the formation of aggregates of the complex or nanoparticles having a size no more than about 200% (such as no more than about any of 190, 180, 170, 160, 150, 140, 130, 120, 110, 100, 90, 80, 70, 60, 50, 40, 30, 20, 10, 9, 8, 7, 6, 5, 4, 3, 2, or 1%, including any ranges between any of these values) larger than the size of the complex or nanoparticle. In some embodiments, the composition comprises a carrier protein (e.g., albumin) at a concentration that promotes and/or contributes to the formation of aggregates of the complex or nanoparticles having a size no more than about 150% larger than the size of the complex or nanoparticle. In some embodiments, the composition comprises a carrier protein (e.g., albumin) at a concentration that promotes and/or contributes to the formation of aggregates of the complex or nanoparticles having a size no more than about 100% larger than the size of the complex or nanoparticle. In some embodiments, the composition comprises a carrier protein (e.g., albumin) at a concentration that promotes and/or contributes to the formation of aggregates of the complex or nanoparticles having a size no more than about 50% larger than the size of the complex or nanoparticle. In some embodiments, the composition comprises a carrier protein (e.g., albumin) at a concentration that promotes and/or contributes to the formation of aggregates of the complex or nanoparticles having a size no more than about 25% larger than the size of the complex or nanoparticle. In some embodiments, the composition comprises a carrier protein (e.g., albumin) at a concentration that promotes and/or contributes to the formation of aggregates of the complex or nanoparticles having a size no more than about 10% larger than the size of the complex or nanoparticle. In some embodiments, the carrier protein is albumin. In some embodiments, the albumin is human serum albumin.

In some embodiments, a pharmaceutical composition as described herein is formulated for intravenous, intratumoral, intraarterial, topical, intraocular, ophthalmic, intraportal, intracranial, intracerebral, intracerebroventricular, intrathecal, intravesicular, intradermal, subcutaneous, intramuscular, intranasal, intratracheal (such as intratracheal instillation), pulmonary, intracavity, nasal or oral administration, or nebulization (NB).

In some embodiments, the pharmaceutical composition is formulated for nebulization. In some embodiments, the pharmaceutical composition is formulated for nasal administration. In some embodiments, the pharmaceutical composition is formulated for intratracheal administration (such as intratracheal instillation). In some embodiments, the pharmaceutical composition is formulated for inhalation. In some embodiments, the pharmaceutical composition is formulated for intravenous administration.

### Methods of Preparation

Peptides described herein, such as any of the chimeric peptides, blocking peptides and non-naturally occurring peptides described herein can be synthesized by any methods known in the field. For example, peptides can be synthesized by solid-phase peptide synthesis using AEDI-expensin resin with (fluorenylmethoxy)-carbonyl (Fmoc) on a Pioneer Peptide Synthesizer (Pioneer™, Applied Biosystems, Foster City, CA). See, for example, WO2014/053628, US20160115199A1 or EP 2928906, which are incorporated herein by reference in their entirety.

Also see Examples (such as Example 1) for methods of preparing peptides described herein. Cyclic peptide preparation is described in, for example, Rashad et al. (Methods Mol Biol. 2019; 2001:133-145) and Alsina J. et al. (Tetrahedron Letters, Volume 35, Issue 51, 19 Dec. 1994, Pages 9633-9636.)

In some embodiments, there is provided a method of preparing a complex or nanoparticle as described herein.

In some embodiments, there is provided a method of preparing the complex comprising a second peptide and a cargo molecule as described above, comprising combining the second peptide with the cargo molecule, thereby forming the complex.

In some embodiments, the peptide and the cargo molecule are combined at a molar ratio from about 1:1 to about 100:1 (such as about between about 1:1 and about 50:1, or about 5:1 to about 20:1), respectively.

In some embodiments, the method comprises mixing a first solution comprising the cargo molecule with a second solution comprising the second peptide to form a third solution, wherein the third solution comprises or is adjusted to comprise i) about 0-5% sucrose, ii) about 0-5% glucose, iii) about 0-50% DMEM, iv) about 0-80 mM NaCl, or v) about 0-20% PBS, and wherein the third solution is incubated to allow formation of the complex. In some embodiments, the first solution comprises the cargo in sterile water and/or wherein the second solution comprises the peptide in sterile water. In some embodiments, the third solution is adjusted to comprise i) about 0-5% sucrose, ii) about 0-5% glucose, iii) about 0-50% DMEM, iv) about 0-80 mM NaCl, or v) about 0-20% PBS after incubating to form the complex.

In some embodiments, the method further comprises a filtration process, wherein the complex is filtered through a pore-sized membrane. In some embodiments, the pore has a diameter of at least about 0.1 μm (such as at least about 0.1 μm, 0.15 μm, 0.2 μm, 0.25 μm, 0.3 μm, 0.35 μm, 0.4 μm, 0.45 μm, 0.5 μm, 0.6 μm, 0.7 μm, 0.8 μm, 0.9 μm, 1.0 μm, 1.1 μm or 1.2 m). In some embodiments, the pore has a diameter of no more about 1.2 μm, 1.0 μm, 0.8 μm, 0.6 μm, 0.5 μm, 0.45 μm, 0.4 μm, 0.35 μm, 0.3 μm, or 0.25 μm. In some embodiments, the port has a diameter of about 0.1 μm to about 1.2 μm (such as about 0.1 to about 0.8 μm, about 0.2 to about 0.5 μm).

In some embodiments, for a stable composition comprising a cargo molecule delivery complex or nanoparticle of the application, the average diameter of the complex or nanoparticle does not change by more than about 10%, and the polydispersity index does not change by more than about 10%.

Also provided are methods of preparing any of the peptides described herein.

### Methods of Use

The present application in one aspect provides a method of treating a virus infection (such as SARS-CoV-2 infection) or a disease or condition associated with the virus infection in an individual, comprising administering to the individual a composition (e.g., a pharmaceutical composition) comprising a chimeric peptide, a blocking peptide, a siRNA, a complex, a nanoparticle, a composition such as those described above. The present application also provides a method of preventing a virus infection (such as SARS-CoV-2 infection) or a disease or condition associated with the virus infection in an individual, comprising administering to the individual a composition (e.g., a pharmaceutical composition) comprising a chimeric peptide, a blocking peptide, a siRNA, a complex, a nanoparticle, a composition such as those described above. In some embodiments, the virus is SARS-CoV-2.

In some embodiments, there is provided a method of treating a virus infection (such as SARS-CoV-2 infection) or a disease or condition associated with the virus infection in an individual, comprising administering to the individual a composition comprising a chimeric peptide comprising a blocking peptide connected to a stabilizing peptide via a linker, wherein the blocking peptide comprises a sequence selected from the group consisting of SEQ ID NOs: 1, 6, 8-11, 23, 24, 26-28, 31 42, 45 and 47-52, wherein the stabilizing peptide comprises an ADGN-100 peptide or a VEPEP-6 peptide. In some embodiments, the linker is selected from the group consisting of a proline, a polyglycine linker moiety, a PEG moiety, Aun, Ava, and Ahx. In some embodiments, the stabilizing peptide comprises the amino acid sequence of SEQ ID NO: 55 or 97. In some embodiments, the blocking peptide comprises a cyclic peptide selected from the group consisting of SEQ ID NOs: 151, 156, and 158-160. In some embodiments, the blocking peptide comprises a sequence selected from the group consisting of SEQ ID NOs: 23, 24, 26-28, 31 and 47-52. In some embodiments, the blocking peptide comprises a sequence selected from the group consisting of SEQ ID NOs: 23, 24, 26-28 and 31. In some embodiments, the chimeric peptide comprises the amino acid sequence set forth in SEQ ID NOs: 17, 20, 21, 27, 28, 33, 39, and 40. In some embodiments, the virus is SARS-CoV-2. In some embodiments, the individual is a human. In some embodiments, the composition is administered intravenously, nasally or intratracheally, or by inhalation or nebulization,

In some embodiments, there is provided a method of treating a virus infection (such as SARS-CoV-2 infection) or a disease or condition associated with the virus infection in an individual, comprising administering to the individual a composition comprising a blocking peptide comprising a non-naturally occurring peptide selected from any one of SEQ ID NOs: 12-22, 24-41, and 151-160. In some embodiments, the blocking peptide comprises the amino acid sequence of any of SEQ ID NOs: 12, 17, 19-22, 24, 26-28, 31-33, 35, 38-40, 151, 156, and 158-160. In some embodiments, the blocking peptide comprises an amino acid sequence of any one of SEQ ID NOs: 27, 38, 39, 40. In some embodiments, the virus is SARS-CoV-2. In some embodiments, the individual is a human. In some embodiments, the composition is administered intravenously, nasally or intratracheally, or by inhalation or nebulization,

In some embodiments, there is provided a method of treating or preventing a virus infection (such as SARS-CoV-2 infection) or a disease or condition associated with the virus infection in an individual, comprising administering to the individual a composition comprising a siRNA comprising the nucleic acid sequence of any one of SEQ ID NOs: 161-170. In some embodiments, the siRNA comprises the nucleic acid sequence selected from the group consisting of SEQ ID NOs 163, 166, 168, and 170. In some embodiments, the siRNA comprises the nucleic acid sequence of SEQ ID NO: 166. In some embodiments, the virus is SARS-CoV-2. In some embodiments, the individual is a human. In some embodiments, the composition is administered intravenously, nasally or intratracheally, or by inhalation or nebulization,

In some embodiments, there is provided a method of treating or preventing a virus infection (such as SARS-CoV-2 infection) or a disease or condition associated with the virus infection in an individual, comprising administering to the individual a composition comprising a complex comprising a) a cargo comprising a first peptide comprising the amino acid sequence set forth in SEQ ID NOs: 12, 17, 19-22, 24, 26-28, 31-33, 35, 38-40, 151, 156, and 158-160 (e.g., SEQ ID NOs: 17, 20, 21, 27, 28, 33, 39, and 40), and b) a second peptide, wherein the second peptide is selected from the group consisting of CADY, MPG, PEP-1 peptides, PEP-2 peptides, PEP-3 peptides (e.g., SEQ ID NO: 183), LNCOV peptides, VEPEP-3 peptides, VEPEP-4 peptides, VEPEP-5 peptides, VEPEP-6 peptides, VEPEP-9 peptides, and ADGN-100 peptides. In some embodiments, the molar ratio of the second peptide to the first peptide is between about 1:1 to about 80:1 (e.g., about 2:1 to about 10:1, about 5:1). In some embodiments, the second peptide is complexed with the first peptide. In some embodiments, the virus is SARS-CoV-2. In some embodiments, the individual is a human. In some embodiments, the composition is administered intravenously, nasally or intratracheally, or by inhalation or nebulization,

In some embodiments, there is provided a method of treating or preventing a virus infection (such as SARS-CoV-2 infection) or a disease or condition associated with the virus infection in an individual, comprising administering to the individual a composition comprising a complex comprising a) a cargo comprising a blocking peptide comprising the amino acid sequence of any one of SEQ ID NOs: 27, 38, 39, 40, and b) a second peptide, wherein the second peptide is selected from the group consisting of CADY, MPG, PEP-1 peptides, PEP-2 peptides, PEP-3 peptides (e.g., SEQ ID NO: 183), LNCOV peptides, VEPEP-3 peptides, VEPEP-4 peptides, VEPEP-5 peptides, VEPEP-6 peptides, VEPEP-9 peptides, and ADGN-100 peptides. In some embodiments, the molar ratio of the second peptide to the blocking peptide is between about 1:1 to about 80:1 (e.g., about 2:1 to about 10:1, about 5:1). In some embodiments, the second peptide is complexed with the blocking peptide. In some embodiments, the virus is SARS-CoV-2. In some embodiments, the individual is a human. In some embodiments, the composition is administered intravenously, nasally or intratracheally, or by inhalation or nebulization,

In some embodiments, there is provided a method of treating or preventing a virus infection (such as SARS-CoV-2 infection) or a disease or condition associated with the virus infection in an individual, comprising administering to the individual a composition comprising a complex comprising a) a cargo comprising a siRNA comprising the nucleic acid sequence selected from the group consisting of SEQ ID NOs 163, 166, 168, and 170 (e.g., SEQ ID NO: 166), and b) a second peptide selected from the group consisting of an ADGN-100 peptide (e.g., SEQ ID NO: 55), VEPEP-6 peptide (e.g., SEQ ID NO: 97) or VEPEP-9 peptide (e.g., SEQ ID NO: 120 or 121), wherein the siRNA is complexed with the second peptide. In some embodiments, the molar ratio of the second peptide to the siRNA is between about 1:1 to about 80:1 (e.g., about 5:1 to about 50:1, about 20:1). In some embodiments, the virus is SARS-CoV-2. In some embodiments, the individual is a human. In some embodiments, the composition is administered intravenously, nasally or intratracheally, or by inhalation or nebulization,

In some embodiments, there is provided a method of treating or preventing a virus infection (such as SARS-CoV-2 infection) or a disease or condition associated with the virus infection in an individual, comprising administering to the individual a composition comprising a complex comprising i) a chimeric peptide comprising the amino acid sequence set forth in SEQ ID NOs: 17, 20, 21, 27, 28, 33, 39, and 40, ii) a siRNA comprising the nucleic acid sequence selected from the group consisting of SEQ ID NOs 163, 166, 168, and 170 (e.g., SEQ ID NO: 166), and b) a second peptide, wherein the second peptide is selected from the group consisting of CADY, MPG, PEP-1 peptides, PEP-2 peptides, PEP-3 peptides (e.g., SEQ ID NO: 183), LNCOV peptides, VEPEP-3 peptides, VEPEP-4 peptides, VEPEP-5 peptides, VEPEP-6 peptides, VEPEP-9 peptides, and ADGN-100 peptides. In some embodiments, the virus is SARS-CoV-2. In some embodiments, the individual is a human. In some embodiments, the composition is administered intravenously, nasally or intratracheally, or by inhalation or nebulization,

In some embodiments, there is provided a method of treating or preventing a virus infection (such as SARS-CoV-2 infection) or a disease or condition associated with the virus infection in an individual, comprising administering to the individual a composition comprising a complex comprising i) a blocking peptide comprising the amino acid sequence of any of SEQ ID NOs: 12, 17, 19-22, 24, 26-28, 31-33, 35, 38-40, 151, 156, and 158-160, ii) a siRNA comprising the nucleic acid sequence selected from the group consisting of SEQ ID NOs 163, 166, 168, and 170 (e.g., SEQ ID NO: 166), and b) a second peptide, wherein the second peptide is selected from the group consisting of CADY, MPG, PEP-1 peptides, PEP-2 peptides, PEP-3 peptides (e.g., SEQ ID NO: 183), LNCOV peptides, VEPEP-3 peptides, VEPEP-4 peptides, VEPEP-5 peptides, VEPEP-6 peptides, VEPEP-9 peptides, and ADGN-100 peptides. In some embodiments, the virus is SARS-CoV-2. In some embodiments, the individual is a human. In some embodiments, the composition is administered intravenously, nasally or intratracheally, or by inhalation or nebulization,

In some embodiments, there is provided a method of treating or preventing a virus infection (such as SARS-CoV-2 infection) or a disease or condition associated with the virus infection in an individual, comprising administering to the individual a composition comprising a) a first complex comprising a chimeric peptide or blocking peptide (such as any of the chimeric peptide or blocking peptide described herein) complexed with a second peptide (such as any of the second peptide described herein), and b) a second complex comprising an siRNA (such as any of the siRNAs described herein) complexed with a third peptide (such as any of the peptides described in “the second peptide” section), wherein the chimeric or blocking peptide inhibits the interaction between ACE2 and SPIKE, and wherein the siRNA targets nucleocapsid of SPIKE. In some embodiments, the chimeric peptide or blocking peptide comprises the amino acid sequence set forth in SEQ ID NOs: 17, 20, 21, 27, 28, 33, 39, and 40. In some embodiments, the siRNA comprises the nucleic acid sequence selected from the group consisting of SEQ ID NOs 163, 166, 168, and 170 (e.g., SEQ ID NO: 166). In some embodiments, the virus is SARS-CoV-2. In some embodiments, the individual is a human. In some embodiments, the composition is administered intravenously, nasally or intratracheally, or by inhalation or nebulization,

In some embodiments of the methods described herein, the individual is a mammal. In some embodiments, the individual is human. In some embodiments, the individual is a male. In some embodiments, the individual is a female. In some embodiments, the individual has a compromised immune system.

In some embodiment, the individual is exposed to the virus (e.g., SARS-CoV-2). In some embodiments, the individual has exhibited first symptom (e.g., a fever, e.g., dry cough, e.g., shortness of breath) of the viral infection (e.g., SARS-CoV-2 infection). In some embodiments, the individual has chest pain or pressure, shortness of breath and/or bluish lips or face. In some embodiments, the individual has acute respiratory distress syndrome (ARDs). In some embodiments, the individual is admitted to the hospital. In some embodiments, the individual enters intensive care unit (ICU).

In some embodiments, the composition is administered within about 1, 3, 5, or 7 days from the exposure to the virus (e.g., SARS-CoV-2). In some embodiments, the composition is administered prior to the exposure to the virus. In some embodiments, the composition is administered about 1, 2, 3, 4, 5, 6, or 7 days prior to a potential exposure to the virus. In some embodiments, the composition is administered about 1, 2, or 3 weeks prior to a potential exposure to the virus.

In some embodiments, the composition is administered within about 1, 3, 5, or 7 from appearance of the first symptom (e.g., a fever, e.g., dry cough, e.g., shortness of breath) of the viral infection (e.g., SARS-CoV-2 infection).

In some embodiments, the composition is administered within about 1, 2, 3, 4, 5, 6, 7 days when the individual has chest pain or pressure, shortness of breath and/or bluish lips or face.

In some embodiments, the composition is administered within about 3, 6, 12, or 24 hours when the individual has acute respiratory distress syndrome (ARDs). In some embodiments, the composition is administered within about 1, 2, 3, 4, or 5 when the individual has acute respiratory distress syndrome (ARDs).

In some embodiments, the composition is administered three times a day, twice a day, daily, once every two, three, four, five, or six days, once weekly, once bi-weekly, or once monthly.

### Virus

In some embodiments, the virus infection is caused by a virus which is a member of Coronaviridae (e.g., alphacoronavirus, betacoronavirus, deltacoronavirus, or gammacoronavirus), Orthomyxoviridae (e.g., an influenza virus), Flaviviridae (e.g., flavivirus or hepacivirus), or Caliciviridae (e.g., norovirus).

In some embodiments, the virus infection is caused by a virus which is a member of Coronaviridae. In some embodiments, the virus is an alphacoronavirus (e.g., HCoV-229E or HCoV-NL63), a betacoronavirus (e.g. HCoV-OC43, HCoV-HKU1, MERS-CoV, SARS-CoV, or SARS-CoV-2), a deltacoronavirus, or a gammacoronavirus.

In some embodiment, the virus is an alphacoronavirus. In some embodiments, the virus is HCoV-229E or HCoV-NL63. In some embodiments, the virus is HCoV-229E. In some embodiments, the virus is HCoV-NL63.

In some embodiments, the virus is a betacoronavirus. In some embodiments, the virus is HCoV-OC43, HCoV-HKU1, MERS-CoV, SARS-CoV, or SARS-CoV-2. In some embodiments, the virus is MERS-CoV, SARS-CoV, or SARS-CoV-2. In some embodiments, the virus is HCoV-OC43. In some embodiments, the virus is HCoV-HKU1. In some embodiments, the virus is HMERS-CoV. In some embodiments, the virus is SARS-CoV.

In some embodiments, the virus is SARS-CoV-2.

### Combination Therapy

In some embodiments, the method also comprises administering a therapeutically effective amount of a second agent or therapy. The second agent or therapy described herein can be any medication or therapy that is useful for treating the virus infection. In some embodiments, the second agent comprises mycophenolate mofetil (MMF) and/or a corticosteroid. In some embodiments, the method comprises administering a therapeutically effective amount of mycophenolate mofetil (MMF). In some embodiments, the method comprises administering a therapeutically effective amount of a corticosteroid.

In some embodiments, the method also comprises administering a therapeutically effective amount of an additional anti-viral agent. In some embodiments, the additional anti-viral agent is remdesivir, lopinavir/ritonavir, IFN-α, lopinavir, ritonavir, penciclovir, galidesivir, disulfiram, darunavir, cobicistat, ASC09F, disulfiram, nafamostat, griffithsin, alisporivir, chloroquine, hydroxychloroquine, nitazoxanide, baloxavir marboxil, oseltamivir, zanamivir, peramivir, amantadine, rimantadine, favipiravir, laninamivir, ribavirin, umifenovir, or any combination thereof. In some embodiments, the anti-viral agent is chloroquine. In some embodiments, the anti-viral agent is hydroxychloroquine. In some embodiments, the anti-viral agent is remdesivir.

In some embodiments, the composition and the second agent are administered simultaneously. In some embodiments, the composition and the second agent are administered concurrently. In some embodiments, the composition and the second agent are administered sequentially.

### Dosing and Methods of Administration

The dosing frequency of the composition and/or the second agent/therapy may be adjusted over the course of the treatment, based on the judgment of the administering physician. When administered separately, the composition and/or the second agent/therapy can be administered at different dosing frequency or intervals. In some embodiments, sustained continuous release formulation of the composition and/or the second agent/therapy may be used. Various formulations and devices for achieving sustained release are known in the art. A combination of the administration configurations described herein can also be used.

In some embodiments, dosages of the chimeric peptide, blocking peptide, or siRNAs described herein for treatment of human or mammalian subjects are in the range of about 0.001 mg/kg to about 100 mg/kg for each administration. In some embodiments, the concentration of the chimeric or blocking peptide described herein at an infection site after administration is about 0.01 μM to 10 μM (such as about 0.1 μM to about 1 μM, such as about 0.5 μM). In some embodiments, the concentration of the siRNA described herein at an infection site after administration is about 0.1 nM to about 10 μM (such as about 1 nM to 1 μM, such about 1 nM to about 200 nM).

In some embodiments, the composition is administered within about 1, 2, 3, 4, 5, 6, 7, 10, 12, 14, 16, 18, 20, 25, or 30 days from the exposure to the virus (e.g., SARS-CoV-2).

In some embodiments, the composition is administered within about 1, 2, 3, 4, 5, 6, 7, 10, 12, 14, 16, 18, or 20 days from appearance of the first symptom (e.g., a fever, e.g., dry cough, e.g., shortness of breath) of the viral infection (e.g., SARS-CoV-2 infection). In some embodiments, the composition is administered when the symptom (e.g., a fever, e.g., dry cough, e.g., shortness of breath) lasts for at least 2, 3, 4, 5, 6, or 7 days.

In some embodiments, the composition is administered within about 1, 2, 3, 4, 5, 6, 7 days when the individual has chest pain or pressure, shortness of breath and/or bluish lips or face.

In some embodiments, the composition is administered within about 3, 6, 12, or 24 hours when the individual has acute respiratory distress syndrome (ARDs). In some embodiments, the composition is administered within about 1, 2, 3, 4, or 5 when the individual has acute respiratory distress syndrome (ARDs).

In some embodiments, the composition is administered three times a day, twice a day, daily, once every two, three, four, five, or six days, once weekly, once bi-weekly, or once monthly.

The composition and/or the second agent/therapy can be administered using the same route of administration or different routes of administration. In some embodiments of the methods described herein, the composition or second agent/therapy described herein is administered to the individual by any of intravenous, intratumoral, intraarterial, topical, intraocular, ophthalmic, intraportal, intracranial, intracerebral, intracerebroventricular, intrathecal, intravesicular, intradermal, subcutaneous, intramuscular, intranasal, intratracheal, pulmonary, intracavity, nasal, or oral administration, or nebulization (NB) or intratracheal instillation.

In some embodiments, the composition and/or the second agent/therapy as described herein is formulated for systemic or tropical administration. In some embodiments, the composition and/or the second agent/therapy as described herein is formulated for intravenous, intratumoral, intraarterial, topical, intraocular, ophthalmic, intraportal, intracranial, intracerebral, intracerebroventricular, intrathecal, intravesicular, intradermal, subcutaneous, intramuscular, intranasal, intratracheal, pulmonary, intracavity, or oral administration, or nebulization (NB), inhalation, or intratracheal instillation.

In some embodiments, the composition and/or the second agent/therapy is formulated for or administered to the individual by nebulization. In some embodiments, the composition and/or the second agent/therapy is formulated for or administered to the individual by nasal administration. In some embodiments, the composition and/or the second agent/therapy is formulated for or administered to the individual by intratracheal administration (such as intratracheal instillation). In some embodiments, the composition and/or the second agent/therapy is formulated for or administered to the individual by inhalation. In some embodiments, the composition and/or the second agent/therapy is formulated for or administered to the individual by intravenous administration.

### Patient Population

In some embodiments, the individual has a compromised immune system.

In some embodiments, the individual is a male. In some embodiments, the individual is a female.

In some embodiments, the individual has an age of at least about 40, 45, 50, 55, 60, 65, 70, 75, 80, 85 or 90 years old. In some embodiments, the individual has an age of no more than 18, 15, 12, 10, 8, 6, 4, 2, or 1 year old. In some embodiments, the individual has an age of about 20 to about 40 years old.

In some embodiment, the individual is exposed to the virus (e.g., SARS-CoV-2).

In some embodiments, the individual has exhibited first symptom (e.g., a fever, e.g., dry cough, e.g., shortness of breath) of the viral infection (e.g., SARS-CoV-2 infection).

In some embodiments, the individual has exhibited chest pain or pressure, shortness of breath and/or bluish lips or face. In some embodiments, the individual has acute respiratory distress syndrome (ARDs). In some embodiments, the individual is admitted to the hospital. In some embodiments, the individual enters intensive care unit (ICU).

In some embodiments, the individual has ACE2 expression (e.g., ACE2 expression on lung cells) higher than a reference ACE2 expression (e.g., at least 10%, 20%, 30%, 40%, 50%, 75%, 100%, 150%, or 200% higher). In some embodiments, the reference ACE2 expression is the average ACE2 expression of a group of individuals that have the same sex and/or ethnicity as the individual.

In some embodiments, the individual disclosed herein has an autoimmune disease. In some embodiments, the individual has a disease or condition associated with transplant rejection.

In some embodiments, the individual has been treated with or failed the treatment of mycophenolate mofetil (MMF). In some embodiments, the individual has been treated with or failed the treatment of a corticosteroid. In some embodiments, the individual has been treated with or failed the treatment of any one or more of remdesivir, lopinavir/ritonavir, IFN-α, lopinavir, ritonavir, penciclovir, galidesivir, disulfiram, darunavir, cobicistat, ASC09F, disulfiram, nafamostat, griffithsin, alisporivir, chloroquine, hydroxychloroquine, nitazoxanide, baloxavir marboxil, oseltamivir, zanamivir, peramivir, amantadine, rimantadine, favipiravir, laninamivir, ribavirin, umifenovir, or any combination thereof. In some embodiments, the individual has been treated with or failed the treatment of chloroquine. In some embodiments, the individual has been treated with or failed the treatment of hydroxychloroquine. In some embodiments, the individual has been treated with or failed the treatment of is remdesivir.

### Kits

Also provided herein are kits, reagents, and articles of manufacture useful for the methods described herein. In some embodiments, kit contains vials containing any of the peptides and siRNAs described herein, such as any of the chimeric peptides, blocking peptides, siRNAs, second peptides (such as cell-penetrating peptides), optionally with other molecules, combined in one vial or separately in different vials. In some embodiments, the second peptide (such as any of the cell-penetrating peptides) are combined accordingly with the appropriate one or more chimeric peptide/blocking peptide and/or siRNAs to result in complexes or nanoparticles that can be administered to the patient for an effective treatment. Thus, in some embodiments, there is provided a kit comprising: 1) a chimeric peptide or blocking peptide described herein, 2) a second peptide. In some embodiments, there is provided a kit comprising: 1) a siRNA described herein, 2) a second peptide. In some embodiments, there is provided a kit comprising: 1) a siRNA described herein, 2) a chimeric peptide or blocking peptide described herein, optionally 3) a second peptide described herein. In some embodiment, the kit further comprises a pharmaceutically acceptable carrier.

The kits described herein may further comprise instructions for using the components of the kit to practice the subject methods (for example instructions for making the pharmaceutical compositions described herein and/or for use of the pharmaceutical compositions). The instructions for practicing the subject methods are generally recorded on a suitable recording medium. For example, the instructions may be printed on a substrate, such as paper or plastic, etc. As such, the instructions may be present in the kits as a package insert, in the labeling of the container of the kits or components thereof (i.e., associated with the packaging or sub packaging) etc. In some embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate

The various components of the kit may be in separate containers, where the containers may be contained within a single housing, e.g., a box.

### Exemplary Embodiments

Embodiment 1. A chimeric peptide comprising a blocking peptide connected to a stabilizing peptide, wherein the blocking peptide specifically blocks interaction between SPIKE and ACE2, and wherein the stabilizing peptide stabilizes secondary or tertiary structure of the blocking peptide.

Embodiment 2. The chimeric peptide of claim 1, wherein the blocking peptide comprises a loop sequence within the receptor-binding domain (RBD) of SPIKE.

Embodiment 3. The chimeric peptide of claim 2, wherein the loop sequence has a length of no more than about 20 amino acids.

Embodiment 4. The chimeric peptide of claim 3, wherein the loop sequence has a length of about 7 amino acids to about 18 amino acids.

Embodiment 5. The chimeric peptide of any one of claims 1-4, wherein the blocking peptide comprises a K at the C-terminus.

Embodiment 6. The chimeric peptide of any one of claims 1-5, wherein the loop sequence is selected from the group consisting of SEQ ID NOs: 1-11 and 42-46.

Embodiment 7. The chimeric peptide of claim 6, wherein the loop sequence is selected from the group consisting of SEQ ID NOs: 1, 6, 8-11, 42 and 45.

Embodiment 8. The chimeric peptide of claim 1, wherein the blocking peptide comprises a sequence derived from a sequence within the extracellular domain of ACE2.

Embodiment 9. The chimeric peptide of claim 8, wherein the blocking peptide comprises a sequence selected from the group consisting of SEQ ID NOs: 23-31 and 47-52.

Embodiment 10. The chimeric peptide of claim 9, wherein the blocking peptide comprises a sequence selected from the group consisting of SEQ ID NOs: 23, 24, 26-28, 31 and 47-52.

Embodiment 11. The chimeric peptide of claim 2-10, wherein the loop sequence is cyclic.

Embodiment 12. The chimeric peptide of any one of claims 1-11, wherein the stabilizing peptide is connected to the C-terminus of the blocking peptide.

Embodiment 13. The chimeric peptide of any one of claims 1-11, wherein the stabilizing peptide is connected to the N-terminus of the blocking peptide.

Embodiment 14. The chimeric peptide of claim 12 or claim 13, wherein the stabilizing peptide has a length of about 12 amino acids to about 30 amino acids.

Embodiment 15. The chimeric peptide of any one of claims 8-14, wherein the blocking peptide and the stabilizing peptide each comprises a sequence derived from ACE2.

Embodiment 16. The chimeric peptide of claim 15, wherein the stabilizing peptide comprises a sequence set forth in SEQ ID NO: 49 or 50.

Embodiment 17. The chimeric peptide of any one of claims 1-14, wherein the stabilizing peptide comprises an amphipathic helix structure.

Embodiment 18. The chimeric peptide of claim 17, wherein the stabilizing peptide comprises an ADGN-100 peptide or a VEPEP-6 peptide.

Embodiment 19. The chimeric peptide of claim 18, wherein the stabilizing peptide comprises a sequence set forth in any one of SEQ ID NOs: 53-107.

Embodiment 20. The chimeric peptide of claim 19, wherein the stabilizing peptide comprises a sequence set forth in SEQ ID NO: 55 or 97.

Embodiment 21. The chimeric peptide of any one of claims 1-20, wherein the blocking peptide and the stabilizing peptide are connected via a linker.

Embodiment 22. The chimeric peptide of claim 21, wherein the linker is selected from the group consisting of a proline, a polyglycine linker moiety, a PEG moiety, Aun, Ava, and Ahx.

Embodiment 23. The chimeric peptide of claim 19, wherein the PEG moiety consists of about two to about seven ethylene glycol units.

Embodiment 24. The chimeric peptide of any one of claims 1-23, wherein the chimeric peptide comprises the amino acid sequence of any one of SEQ ID NOs: 12-22, 27, 28, and 31-41.

Embodiment 25. The chimeric peptide of claim 24, wherein the chimeric peptide comprises the amino acid sequence selected from the group consisting of SEQ ID NOs: 12, 17, 19-22, 27, 28, 31-33, 35, and 38-40.

Embodiment 26. A non-naturally occurring peptide comprising the amino acid sequence of any of SEQ ID NOs: 12-22, 24-41, and 151-160.

Embodiment 27. The non-naturally occurring peptide of claim 26, wherein the peptide comprises the amino acid sequence of any of SEQ ID NOs: 12, 17, 19-22, 24, 26-28, 31-33, 35, 38-40, 151, 156, and 158-160.

Embodiment 28. The non-naturally occurring peptide of embodiment 26 or 27, wherein the peptide has a length of no more than about 100 amino acids.

Embodiment 29. A siRNA comprising a nucleic acid sequence selected from the group consisting of SEQ ID NOs: 161-180.

Embodiment 30. The siRNA of embodiment 29, wherein the siRNA comprises a nucleic acid sequence set forth in SEQ ID NO: 163, 170, 166 or 168.

Embodiment 31. A complex comprising a) a cargo comprising the chimeric peptide of any of the embodiments 1-25, the peptide of embodiment 26 or 27, or A siRNA of embodiment 28 or embodiment 29, and b) a second peptide, wherein the peptide or siRNA is complexed with the second peptide.

Embodiment 32. The complex of embodiment 31, wherein the second peptide is a cell penetrating peptide selected from the group consisting of CADY, PEP-1 peptides, PEP-2 peptides, PEP-3 peptides, LNCOV peptides, VEPEP-3 peptides, VEPEP-6 peptides, VEPEP-9 peptides, and ADGN-100 peptides.

Embodiment 33. The complex of embodiment 31 or embodiment 32, wherein the molar ratio of the second peptide to the peptide or siRNA is between about 1:1 and about 80:1.

Embodiment 34. The complex of embodiment 33, wherein the molar ratio of the second peptide to the peptide is between about 2:1 to about 10:1.

Embodiment 35. The complex of embodiment 34, wherein the molar ratio of the second peptide to the siRNA is between about 5:1 to about 50:1.

Embodiment 36. The complex of any one of embodiments 31-35, wherein the complex comprises a) the chimeric peptide of any of the embodiments 1-25, or the peptide of any of the embodiments 26-28, and/or b) a siRNA of embodiment 29 or embodiment 30.

Embodiment 37. The complex of embodiment 36, wherein the siRNA comprises a nucleic acid sequence set forth in SEQ ID NO: 166.

Embodiment 38. The complex of embodiment 37, wherein the complex comprises a chimeric peptide comprising the amino acid sequence set forth in SEQ ID NO: 17 or 33.

Embodiment 39. A nanoparticle comprising the complex of any one of embodiments 30-38.

Embodiment 40. The nanoparticle of embodiment 39, wherein the nanoparticle has a diameter of no more than about 100 nm.

Embodiment 41. The nanoparticle of embodiment 40, wherein the nanoparticle has a diameter of about 40 to about 60 nm.

Embodiment 42. A pharmaceutical composition comprising a) the chimeric peptide of any of the embodiments 1-25, the peptide of any of the embodiments 26-28, the siRNA of embodiment 29 or embodiment 30, the complex or the nanoparticle of any of embodiments 31-41, and b) a pharmaceutically acceptable carrier.

Embodiment 43. The pharmaceutical composition of embodiment 42, wherein the composition comprises two or more complexes or nanoparticles, wherein the two or more complexes or nanoparticles comprise different cargos.

Embodiment 44. A method of preparing the complex or the nanoparticle of any one of embodiments 31-41, comprising combining the cargo with the second peptide.

Embodiment 45. A method of treating a SARS-CoV-2 infection in an individual, comprising administering to the individual an effective amount of the pharmaceutical composition of embodiment 42 or embodiment 43.

Embodiment 46. The method of embodiment 45, wherein the pharmaceutical composition is administered via nebulization or local lung or nasal delivery.

Embodiment 47. The method of embodiment 45 or embodiment 46, wherein the individual is a human.

### EXAMPLES

The examples below are intended to be purely exemplary of the application and should therefore not be considered to limit the application in any way. The following examples and detailed description are offered by way of illustration and not by way of limitation.

### Example 1. Design of Peptides and Peptide-Based Nanoparticles Targeting ACE2/SARS CoV RBD Interaction

Peptide inhibitors targeting either ACE2 or Spike Protein binding domains were designed based on both the released crystal (PDB code: 6M17) and electron microscopy (PDB code: 6LZG) structures of the ACE2: Spike protein complexes. Inhibitors were designed in order to obtain stable structural entities that can be further stabilized by either cyclization, stapled chemistry, retro-inverso or the use of D amino acids.

A. Selection of Inhibitor Derived from Spike Protein.

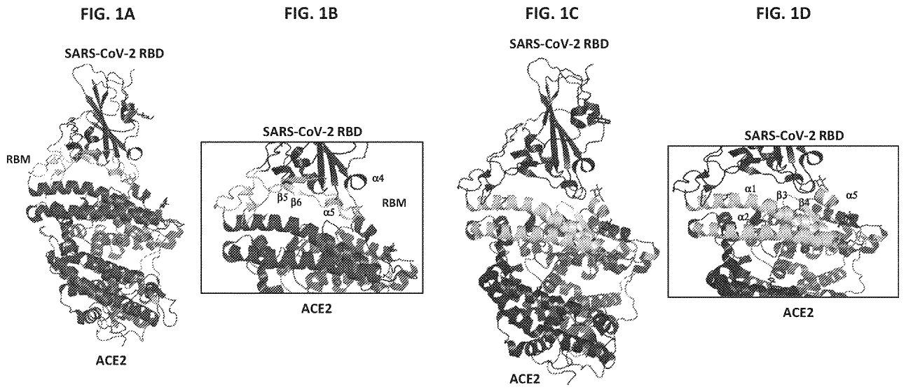

The SARS-CoV-2 RBD structure contained a core domain with five-stranded antiparallel β sheet (β1, β2, β3, β4 and β7) short connecting helices and loops. Most of the contacting residues of SARS-CoV-2 RBD that bind to ACE2 receptor are located in the between the β4 and β7 strands of the core domain (FIG. 1A). The receptor-binding motif (RBM) domain contains the short β5 and β6 strands, α4 and α5 helices and loops. Major contacts involved the loop between α4 and β5, the loop between β6 and α5 and part of the loop between β5 and β6. The cysteine pair Cys480-Cys488, which connects the loops at the distal end of the RBM domain plays also a major role in the stabilization of the interface (FIG. 11B). Eleven inhibitory sequences have been derived from the RBM domain within the SARS-CoV2 RBD structure with the following sequences. See Table 2 below.

TABLE 2

| Sequence | RBM structure motif | Seq code |
|---|---|---|
| SKVGGNYNYLYR | loop α4-β5 | Seq1 (LNCOV-01) |
| KVGGNYNYL | loop α4-β5 | Seq2 (LNCOV-0) |
| GNYNYLYRL | loop α4-β5 | seq3 (LNCOV-1) |
| GGNYNYLYRLFRK | loop α4-β5/β5 | Seq4 (LNCOV-2) |
| STEYQAGST | loop β5-β6 | Seq5 (LNCOV-3) |
| CNGVEGFNC | loop β5-β6 | Seq6 (LNCOV-4) |
| VEGFNCYFP | loop β5-β6 | Seq7 (LNCOV-5) |
| YFPLQSYGFQPTN | B6/loop β6-α5/α5 | Seq8 (LNCOV-6) |
| GVGYQ | | |
| SYGFQPTNGV | loop β6-α5 | Seq9 (LNCOV-7) |
| GFQPTNGVK | loop β6-α5 | Seq10 (LNCOV-8) |
| TNGVGYQPY | loop β6-α5/α5 | Seq11 (LNCOV-9) |

Peptide sequences were coupled to ADGN-106 or ADGN-100 peptides, either as linear or cyclic forms using click chemistry. See Rashad et al., Methods Mol Biol. 2019; 2001:133-145. In some cases, the one or two amino acids at the C-terminus of the blocking peptide is replaced with a lysine to obtain a cyclic peptide via lysine side-chain anchoring. See Alsina J. et al., Tetrahedron Letters, Volume 35, Issue 51, 19 Dec. 1994, Pages 9633-9636. Sequences were directly linked at the N-terminus of ADGN-peptide with an amino bond or coupled via a linker motifs. Ava (4 pentynoic acid), Ahx (aminohexanoic acid), poly-G or PEG (PEG-2) were used as linker. See Table 3.

TABLE 3

| | |
|---|---|
| Ac-S*KVGGNYNYLY*R Ava/Ahx-LWRALWRLWRSLW | Seq12 |
| RLLWK-NH2 | (LNCOV-10) |
| Ac-K*VGGNYNYK*-Ava-LWRALWRLWRSLWRLLWK- | Seq13 |
| NH2 | (LNCOV-11) |
| Ac-G*NYNYLYRL*-Ava-LWRALWRLWRSLWRLLWK- | seq14 |
| NH2 | (LNCOV-12) |
| Ac-G*NYNYLYRLFK*-Ava-LWRALWRLWRSLWRLLW | Seq15 |
| K-NH2 | (LNCOV-22) |
| Ac-T*EYQAGSTK*-Ava-LWRALWRLWRSLWRLLWK- | Sed16 |
| NH2 | (LNCOV-17) |
| Ac-N*GVEGFNK*-Ava-LWRALWRLWRSLWRLLWK- | Seq 17 |
| NH2 | (LNCOV-15) |
| Ac-V*EGENCYF*-Ava-LWRALWRLWRSLWRLLWK- | Seq18 |
| NH2 | (LNCOV-16) |
| Ac-YFPLQSYGFQPTNGVGYQ-Ava-LWRALWRLWRSLW | Seq19 |
| RLLWK-NH2 | (LNCOV-23) |
| Ac-S*YGFQPTNGV*-Ava-LWRALWRLWRSLWRLLWK- | Seq20 |
| NH2 | (LNCOV-24) |
| Ac-G*FQPTNGVK*-Ava-LWRALWRLWRSLWRLLWK- | Seq21 |
| NH2 | (LNCOV-13) |
| Ac-T*NGVGYQPYK*-Ava-LWRALWRLWRSLWRLLWK- | Seq22 |
| NH2 | (LNCOV-14) |

B. Selection of Inhibitor Derived from ACE2 Domain

The extended RBM in the SARS-CoV-2 RBD contacts the bottom side of the small lobe of ACE2, with a concave outer surface in the RBM that accommodates the N-terminal helix of the ACE2. The major contacts between the SARS-CoV-2 RBD and the ACE2 receptor domain involves mainly α1 helix and to minor extends α2 helix and the loop between β33 and β34 strands (FIGS. 1C & 1D).

Nine inhibitory sequences have been selected based on the ACE2 structure with the following sequences. LNCOV-19/33/34 derived from the α1 helix, LNCOV 20/27 from both α1 and α2 helices and LNCOV-35/36/37 from loop between β3 and β4 strands, including part of β3 and β4 strands and the following α helix. See Table 4.

TABLE 4

| | ACE2 | |
|---|---|---|
| | structure | |
| Sequence | motif | Seq code |
| Ac-IEEQAKTFLDKFNHEAEDLFYQS | α1 | Seq23 |
| | | (SBP-1) |
| Ac-EQAKTFLDKFNHEAEDLF | α1 | Seq24 |
| | | (LNCOV-19) |
| Ac-KTFLDKFNHEAEDLF | α1 | Seq25 |
| | | (LNCOV-33) |
| Ac-KTFLDKFNHEAEDLFYS | α1 | Seq26 |
| | | (LNCOV-34) |
| Ac-EQAKTFLDKFNHEAEDLFPKWSAFLK | α1 and α2 | Seq27 |
| EQSTLAQMYG | | (LNCOV-27) |
| Ac-KTFLDKFNHEAEDLFPGPKWSAFLKE | α1 and α2 | Seq28 |
| QSTLAQMYG | | (LNCOV-20) |
| Ac-TAWDLGKGDFRI | β3 and β4 | Seq29 |
| | | (LNCOV-35) |
| Ac-NMTQGFWENSMLTD | β3 and β4 | Seq30 |
| | | (LNCOV-36) |
| Ac-NMTQGFWENPGGPTAWDLGK | β3 and β4 | Seq31 |
| | | (LNCOV-37) |

Peptide sequences were coupled to ADGN peptides, as linear form using click chemistry. Sequences were directly linked at the N-terminus of ADGN-peptide with an amino bond or coupled via a linker motifs. Ava (4 pentynoic acid), Ahx (aminohexanoic acid), poly-G or PEG (PEG-2) were used as linker. See Table 5.

TABLE 5

| | |
|---|---|
| Ac-IEEQAKTFLDKFNHEAEDLFYQS-Ahx-LWRA | Seq32 |
| LWRLWRSLWRLLWK-NH2 | (LNCOV-21) |
| Ac-EQAKTFLDKFNHEAEDLF-Ahx-LWRALWRLW | Seq33 |
| RSLWRLLWK-NH2 | (LNCOV-18) |
| Ac-KTFLDKFNHEAEDLF-Ahx-LWRALWRLWRSL | Seq34 |
| WRLLWK-NH2 | (LNCOV-25) |
| Ac-KTFLDKFNHEAEDLFYS-Ahx-LWRALWRLWR | Seq35 |
| SLWRLLWK-NH2 | (LNCOV-26) |
| AC-TAWDLGKGDFRI-Ahx-LWRALWRLWRSLWRL | Seq36 |
| LWK-NH2 | (LNCOV-28) |
| Ac-NMTQGFWENSMLTD-Ahx-LWRALWRLWRSLW | Seq37 |
| RLLWK-NH2 | (LNCOV-29) |
| Ac-NMTQGFWENPGGPTAWDLGK-Ahx-LWRALWR | Seq38 |
| LWRSLWRLLWK-NH2 | (LNCOV-30) |
| Ac-EQAKTFLDKFNHEAEDLFPKWSAFLKEQSTLA | Seq39 |
| QMYG-Ahx-LWRALWRLWRSLWRLLWK-NH2 | (LNCOV-31) |
| Ac-KTFLDKFNHEAEDLFPKWSAFLKEQSTLAQMY | Seq40 |
| G-Ahx-LWRALWRLWRSLWRLLWK-NH2 | (LNCOV-32) |
| Ac-EQAKTFLDKFNHEAEDLF-ahx-RSAGWRWRL | Seq41 or 188 |
| WRVRSWSR-NH2 | (LNCOV-33) |

### Example 2. Structural Analysis of the Selected Inhibitory Sequences

Secondary structures of the inhibitory sequences derived from the α1 and α2 helices of ACE2 linked or not to ADGN peptides were determined using the molecular modeling peplook-Zultim and PEPFOLD program (Thomas A and Brasseur R., 2006, Prediction of peptide structure: how far are we?, Proteins. 65, 889-97 and Lamiable A, Thévenet P, Rey J, Vavrusa M, Derreumaux P, Tuffery P. Nucleic Acids Res. 2016 Jul. 8; 44 (W1):W449-54).

As reported in Table 6 below, peptides derived from α1 helix (Seq23 to 26) and from α1 and α2 helices (Seq27/28) adopt a secondary helical conformation in solution.

TABLE 6

| Peptide sequences | Seq code |
|---|---|
| Ac-I[EEQAKTFLDKFNHEAEDL]FYQS | Seq23 |
| | (SBP-1) |
| Ac-EQ[AKTFLDKFNHEAED]LF | Seq24 |
| | (LNCOV-19) |
| Ac-KT[FLDKFNHEA]EDLF | Seq25 |
| | (LNCOV-33) |
| Ac-KTF[LDKFNHEAE]DLFYS | Seq26 |
| | (LNCOV-34) |
| Ac-EQA[KTFLDKFNHEAED]LFPKW[SAFLKEQ] | Seq27 |
| ST[LAQM]YG | (LNCOV-27) |
| Ac-KT[FLDKFNHEAED]LFPGPKW[SAFLKEQ]S | Seq28 |
| T[LAQM]YG | (LNCOV-20) |
| Ac-I[EEQAKTFLDKFNHEAEDLFY]QS-Ahx-L | Seq32 |
| [WRA]L[WRLWR]S[LWRLL]WK-NH2 | (LNCOV-21) |
| Ac-EQA[KTFLDKFNHEAEDL]F-Ahx-L[WRA]L | Seq33 |
| [WRLWR]S[LWRLL]WK-NH2 | (LNCOV-18) |
| Ac-KT[FLDKFNHEAE]DLF-Ahx-L[WRA]L[WR | Seq34 |
| LWR]S[LWRLLW]K-NH2 | (LNCOV-25) |
| Ac-KT[FLDKFNHEAEDL]FYS-Ahx-L[WRA]L | Seq35 |
| [WRLWR]S[LWRLLW]K-NH2 | (LNCOV-26) |
| Ac-EQ[AKTFLDKFN]HE[AEDL]FPK[WSAFLKE | Seq39 |
| QSTLAQMY]G-Ahx-L[WRA]L[WRLWR]S[LWRL | |
| LW]K-NH2 | (LNCOV-31) |
| Ac-K[TFLDKFNHEAEDL]FPK[WSAFLKEQSTLA | Seq40 |
| QMY]G-Ahx-L[WRA]L[WRLWR]S[LWRLLW]K- | |
| NH2 | (LNCOV-32) |
| Ac-IE[EQAKTFLDKFNHEAEDLF]YQS-Ahx-[R | Seq41 or 188 |
| SAGWRWRLWRVR]SWSR-NH2 | (LNCOV-33) |

*[ ]: helix motif

Structural dynamic and stability analysis was determined for each helical peptides using Peplook-Zultim program. The Root-Mean-Square Deviation (RMSD) for the amino acids in each peptides and for the whole peptide sequences as well as the interaction energies both van der Waals (vdW) and electrostatics were calculated. The results demonstrated that when coupled to ADGN peptides the helical structure of the inhibitory domain is strongly stabilized, by direct interactions with aromatic residues of the ADGN peptide.

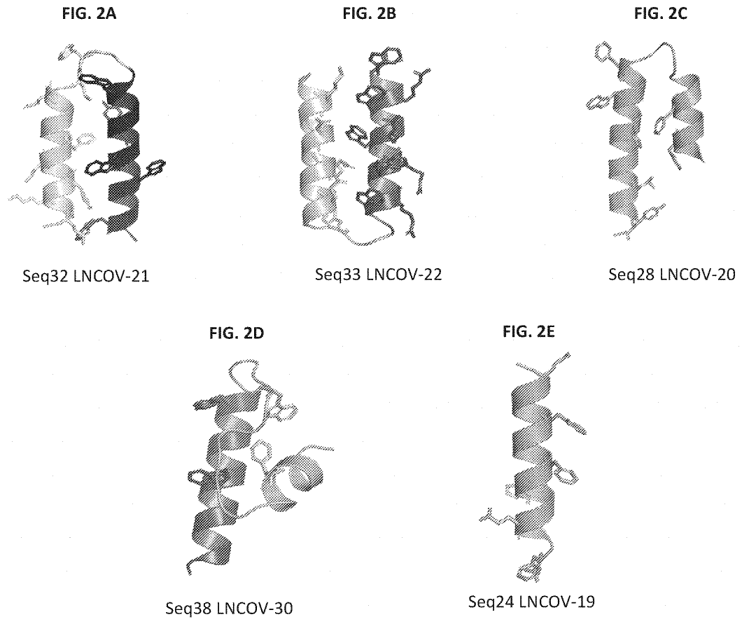

ADGN mediated secondary structure stabilization is mainly observed in Seq 32/33/34/35 and Seq41. The ADGN-106 residues and ADGN-100 residues stabilized the helical structure of the inhibitory domain. The fact that Arginine residues of the ADGN peptide remain accessible at the surface of the peptide allowed further interactions with other ADGN peptide. The residues of the peptides contacting the RBD domain remain accessible at the surface of the peptide (FIGS. 2A-2E).

### Example 3. Screening of Peptides Inhibitors of the ACE2:SARS-CoV-2 Spike Interaction

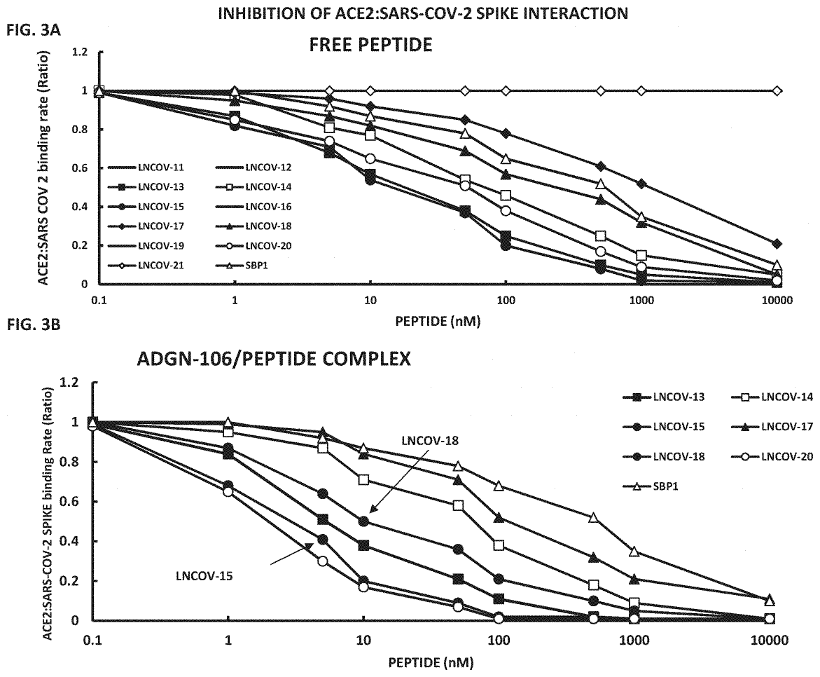

The potency of the different peptides to block the ACE2:SARS-CoV-2 Spike interaction was evaluated in vitro using the ACE2:SARS-CoV-2 Spike Inhibitor Screening Assay Kit (DBS Bioscience). The screening was designed for screening and profiling inhibitors of the ACE2:SARS-CoV-2 Spike interaction. Evaluation was performed in 96-well format. The ACE2 protein was attached to a nickel-coated 96-well plate, then, incubated with peptide inhibitor solutions at room temperature for 30 min with slow shaking. Finally the SARS-CoV-2 Spike-Fc was added to the ACE2 on the plate in the presence of peptide inhibitor solutions and incubate at room temperature for one hour with slow shaking. The plates were treated with Anti-Fc-HRP followed by addition of an HRP substrate to produce chemiluminescence and analyzed using a chemiluminescence reader. SBP-1 peptide described by Zhang et al (The first-in-class peptide binder to the SARS-CoV-2 spike protein, G. Zhang, S. Pomplun, A. R. Loftis, A. Loas, B. L. Pentelute bioRxiv 2020.03.19.999318; doi: https://doi.org/10.1101/2020.03.19.999318 2020) was used a positive control. The results correspond to an average of 3 separate experiments and are reported in FIGS. 3A-3B and Table 7.

SBP-1 control sequence derived from α1 helix of ACE2 prevents ACE2: spike inhibition with an IC₅₀ of 785±25 nM. When SBP-1 is covalently linked to ADGN-106 (Seq 32) the potency is increased by 2.4 folds with IC₅₀ of 325.2±17 nM, confirming that the presence of ADGN-106 stabilized the helical structure of SBP-1 domain. An increase of 1.8 folds of SBP-1 potency was also obtained when covalently linked to ADGN-100 (Seq 41) with IC₅₀ of 432.5±21 nM.

A. Peptides Derived from the Spike Protein:

None of the linear sequences (Seqs 1 to 11) showed inhibition at concentration below 10 μM.

In contrast, Seqs 12/17/19/20/21 peptides which correspond to cyclic peptide covalently linked to ADGN-106 blocked ACE2:SARS-CoV-2 Spike interaction. These results suggested that the presence of cyclization to stabilize the peptide structure is required for the interaction with ACE2 protein surface.

The fact that Seq 8 only inhibits ACE2:SARS-CoV-2 Spike interaction, when covalently linked to ADGN-106 (Seq-19) with IC₅₀ of 90.6±8 nM, confirms that the covalent coupling to ADGN peptide stabilizes the structure of the inhibitory domain.

Seq17 peptide derived from the loop β5-β6, stabilized by the cysteine pair Cys480-Cys488, blocks interaction with IC₅₀ of 57.5±11 nM which is 15 and 6 folds more potent than free SBP-1 or SBP-1 coupled to ADGN-106.

The strongest inhibition was obtained with Seq20 and seq21 peptides derived from the loop between β6-α5 with IC₅₀ of 17.2±5 nM and 22.6±7 nM, which is 40 and 16 folds more potent than free SBP-1 or SBP-1 coupled to ADGN-106.

B. Peptide Derived from ACE2 Protein:

Peptide derived from α1 helix of ACE2, Seq23 to 26 blocked ACE2:SARS-CoV-2 Spike interaction with an IC₅₀ between 785<x<2 μM. The covalent coupling of these peptides to ADGN-106, Seq32-35, improves efficiency by 3-6 folds. Seq33 which corresponds to Seq24 sequence linked to ADGN-106 shows an IC₅₀ of 178±14 nM, in comparison to IC50 of 1.3 μM obtained for Seq24. These results confirming that the presence of ADGN-106 stabilized the helical structure of the inhibitory domain.

The strongest inhibition was obtained with peptides derived from α1 and α2 helices of ACE2; Seq27 and Seq28 blocked ACE2:SARS-CoV-2 Spike interaction with IC₅₀ of about 50 nM, which is 15 and 6 folds more potent than free SBP-1 or SBP-1 coupled to ADGN-106. The covalent coupling of these peptides to ADGN-106, Seqs 39 and 40, increased by 1.4 folds efficiency.

Peptide derived from the β3 and β4 loop of ACE2 protein, Seqs 29/30/31 do not showed inhibition at concentration below 10 μM. The covalent coupling of these peptides to ADGN-106, Seq36-38, improves efficiency by 10-100 folds. Seq38 which corresponds to Seq31 sequence linked to ADGN-106 shows an IC₅₀ of 214.2±21 nM, in comparison to IC₅₀ of 25 μM obtained for Seq31. As for the Spike derived inhibitors, these results demonstrated that the covalent coupling to ADGN peptide is essential to stabilize the structure of these inhibitory peptides.

In conclusion, the best candidates to block ACE2:SARS-CoV-2 Spike interaction correspond to
A cyclic peptide corresponding to the β6-α5 loop of SPIKE covalently linked to ADGN-106 (e.g., Seq20 & Seq21)
A cyclic peptide corresponding to the β5-β6 loop of SPIKE covalently linked to ADGN-106 (e.g., Seq17)
A peptide derived from α1 and α2 helices of ACE2 coupled or not to ADGN-106 (e.g., Seq27, 28, 39, 40).

### Example 4. Screening of Peptides Inhibitors of the SARS-CoV-2 Spike: ACE2 Interaction

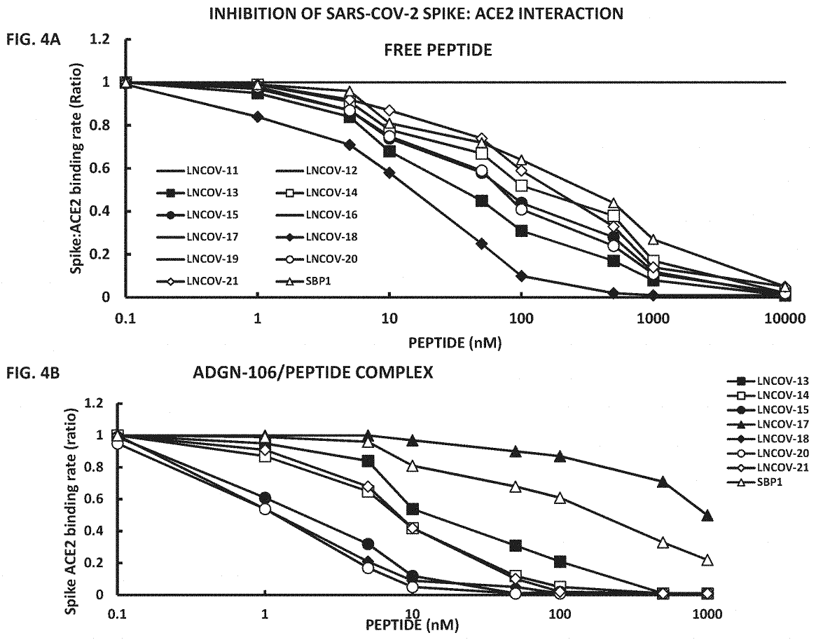

The potency of the different peptides to block the SARS-CoV-2 Spike: ACE2 interaction was evaluated in vitro using the SARS-CoV-2 Spike: ACE2 Inhibitor Screening Assay Kit (DBS Bioscience); The screening was designed for screening and profiling inhibitors of the SARS-CoV-2 Spike: ACE2 interaction. The evaluation was performed in 96-well format. The SARS-CoV-2 Spike protein is attached to a nickel-coated 96-well plate, then, incubated with peptide inhibitor solutions at room temperature for 30 min with slow shaking. Finally the ACE2-Fc is added to the SARS-CoV-2 Spike-Fc on the plate in the presence of peptide inhibitor solutions and incubate at room temperature for one hour with slow shaking. The plates were treated with Anti-Fc-HRP followed by addition of an HRP substrate to produce chemiluminescence and analyzed using a chemiluminescence reader. SBP-1 peptide described by Zhang et al. was used a positive control. The results correspond to an average of 3 separate experiments and are reported in FIGS. 4A-4B and Table 7.

SBP-1 control sequence prevents ACE2: spike inhibition with an IC₅₀ of 267±12 nM. When SBP-1 is covalently linked to ADGN-106 (Seq 32) or ADGN-100 (Seq 41) the potency is increased by 2.0 and 1.4 folds with IC₅₀ of 134.2±14 nM and 219.7±9 nM.

A. Peptides Derived from the Spike Protein

None of the corresponding linear or uncoupled to ADGN-106 sequences (Seqs 1 to 11) showed inhibition at concentration below 10 μM. None of the peptides corresponding to the loop between α4-β5 coupled to ADGN-106 sequences (Seqs 1 to 11) showed inhibition at concentration below 10 μM.

Seqs 17/19/20/21/22 peptides which correspond to cyclic peptide covalently linked to ADGN-106 blocked SARS-CoV-2 Spike: ACE2 interaction which suggested that both the presence of cyclization and the covalent coupling to ADGN peptide stabilize the structure of the inhibitory domain as a major requirement for the interaction with ACE2 protein surface.

The best inhibitions are obtained with Seq20 and seq21 derived from the loop between β36-α5 with IC₅₀ of 44.2±7 nM and 38.5±11 nM, which is 6.6 and 3.3 folds more potent than free SBP-1 or SBP-1 coupled to ADGN-106. Seq22 corresponding to the loop between β6-α5 and α5 helix is 3.6 fold less potent than Seq20/Seq21 suggesting that the loop between β6-α5 will be enough to block the SARS-CoV-2 Spike: ACE2 interaction.

Seq17 derived from the loop β5-β6 inhibits interaction with IC₅₀ of 78.2±9 nM which is 3.4 and 1.7 folds more potent than free SBP-1 or SBP-1 coupled to ADGN-106.

B. Peptide Derived from ACE2 Protein

Peptide derived from α1 helix of ACE2, Seq23 to 26 blocked SARS-CoV-2 Spike: ACE2 interaction with an IC₅₀ between 300<x<1 μM. The covalent coupling of these peptides to ADGN-106, Seq32-35, improves efficiency by 2-10 folds.

The best inhibition was obtained with Seq 33, corresponding to part of the al helix of ACE2 coupled to ADGN-106, which blocked SARS-CoV-2 Spike: ACE2 interaction with IC₅₀ of 18.4±3 nM, in comparison to IC50 of 570±30 nM obtained for Seq24. These results confirming that the presence of ADGN-106 stabilized the helical structure of the inhibitory domain. Seq33 is 14 and 8 folds more potent than free SBP-1 and SBP-1 coupled to ADGN-106.

Peptides derived from α1 and α2 helices of ACE2; Seq27 and Seq28 blocked SARS-CoV-2 Spike: ACE2 interaction with IC₅₀ of 37.4±8 nM and 38.5±7 nM, which is 7 and 3.5 folds more potent than free SBP-1 and SBP-1 coupled to ADGN-106.

Peptide derived from the 33 and 04 loop of ACE2 protein, Seqs 29/30/31 did not show inhibition at concentration below 10 μM. The covalent coupling of these peptides to ADGN-106, Seq36-38, improves efficiency by 10-100 folds. Seq38 which corresponds to Seq31 sequence linked to ADGN-106 shows an IC₅₀ of 102.8±17 nM, in comparison to IC₅₀ of 25 μM obtained for Seq31. As for the Spike derived inhibitors, these results demonstrated that the covalent coupling to ADGN peptide is essential to stabilize the structure of inhibitory peptides.

In conclusion, the best candidates to block SARS-CoV-2 Spike: ACE2 interaction correspond to:
A cyclic peptide corresponding to the β6-α5 loop covalently linked to ADGN-106 (e.g., Seq20 & Seq21)
A peptide derived from α1 helix of ACE2 covalently linked to ADGN-106 (e.g., Seq33)
A peptide derived from α1-α2 helices of ACE2 coupled or not to ADGN-106 (e.g., Seq27, 28, 39, 40).

TABLE 7

| | | | | SARS-CoV-2 | | ACE2:SARS- | |
|---|---|---|---|---|---|---|---|
| | | | | Spike:ACE2 | | CoV-2 Spike | |
| CODE SEQUENCE | ADGN* | CYCLIC** | TARGET | IC₅₀ | | IC₅₀ | |
| Seq1 (LNCOV-01) | − | − | ACE2 | >10 | μM | >10 | μM |
| Seq2 (LNCOV-0) | − | − | ACE2 | >10 | μM | >10 | μM |
| seq3 (LNCOV-1) | − | − | ACE2 | >10 | μM | >10 | μM |
| Seq4 (LNCOV-2) | − | − | ACE2 | >10 | μM | >10 | μM |
| Seq5 (LNCOV-3) | − | − | ACE2 | >10 | μM | >10 | μM |
| Seq6 (LNCOV-4) | − | − | ACE2 | >10 | μM | >10 | μM |
| Seq7 (LNCOV-5) | − | − | ACE2 | >10 | μM | >10 | μM |
| Seq8 (LNCOV-6) | − | − | ACE2 | >10 | μM | >10 | μM |
| Seq9 (LNCOV-7) | − | − | ACE2 | >10 | μM | >10 | μM |
| Seq10 (LNCOV-8) | − | − | ACE2 | >10 | μM | >10 | μM |
| Seq11 (LNCOV-9) | − | − | ACE2 | >1 | μM | >1 | μM |
| Seq12 (LNCOV-10) | + | + | ACE2 | 247 | nM | >1 | μM |
| Seq13 (LNCOV-11) | + | + | ACE2 | >10 | μM | >10 | μM |
| seq14 (LNCOV-12) | + | + | ACE2 | >10 | μM | >10 | μM |
| Seq15 (LNCOV-22) | + | + | ACE2 | >1 | μM | >10 | μM |
| Seq16 (LNCOV-17) | + | + | ACE2 | >1 | μM | >10 | μM |
| Seq17 (LNCOV-15) | + | + | ACE2 | 57.5 | nM | 78.2 ± 14 | nM |
| Seq18 (LNCOV-16) | + | + | ACE2 | >10 | μM | >10 | μM |
| Seq19 (LNCOV-23) | + | − | ACE2 | 90.6 | nM | 348.9 ± 26 | nM |
| Seq20 (LNCOV-24) | + | + | ACE2 | 17.2 | nM | 44.2 ± 7 | nM |
| Seq21 (LNCOV-13) | + | + | ACE2 | 22.6 | nM | 38.5 ± 11 | nM |
| Seq22 (LNCOV-14) | + | + | ACE2 | >1 | μM | 145 | nM |
| Seq23 (SBP-1) | − | − | SPIKE | 785 ± 25 | nM | 267 | nM |
| Seq24 (LNCOV-19) | − | − | SPIKE | 1.3 | μM | 570 ± 30 | μM |
| Seq25 (LNCOV-33) | − | − | SPIKE | 2.5 | μM | 1.9 | μM |
| Seq26 (LNCOV-34) | − | − | SPIKE | 1.9 | μM | 2.1 | μM |
| Seq27 (LNCOV-27) | − | − | SPIKE | 66.1 ± 12 | nM | 37.4 | nM |
| Seq28 (LNCOV-20) | − | − | SPIKE | 58.7 ± 9 | nM | 38.6 | nM |
| Seq29 (LNCOV-35) | − | − | SPIKE | >10 | μM | >10 | μM |
| Seq30 (LNCOV-36) | − | − | SPIKE | >10 | μM | >10 | μM |
| Seq31 (LNCOV-37) | − | − | SPIKE | >10 | μM | >10 | μM |
| Seq32 (LNCOV-21) | + | − | SPIKE | 325.2 ± 17 | μM | 134 | nM |
| Seq33 (LNCOV-18) | + | − | SPIKE | 178 ± 14 | nM | 18.4 | nM |
| Seq34 (LNCOV-25) | + | − | SPIKE | >1 | μM | >1 | μM |
| Seq35 (LNCOV-26) | + | − | SPIKE | 545.8 | nM | 728.5 | nM |
| Seq36 (LNCOV-28) | + | − | SPIKE | >1 | μM | >1 | μM |
| Seq37 (LNCOV-29) | + | − | SPIKE | >1 | μM | 645 | nM |
| Seq38 (LNCOV-30) | + | − | SPIKE | 214.2 | nM | 102.8 ± 17 | nM |
| Seq39 (LNCOV-31) | + | − | SPIKE | 55.2 | nM | 68.4 ± 10 | nM |
| Seq40 (LNCOV-32) | + | − | SPIKE | 45.1 | nM | 85.4 ± 12 | nM |

ADGN* peptide covalently linked to ADGN-106 or ADGN-100
Cyclic* peptide showing a cyclic conformation-linked to ADGN-106

### Example 5. ADGN Peptides Based Nanoparticle Formation with Inhibitor Peptides

We then tested the ability of the different peptides to form nanoparticle organization in the presence of ADGN-106 or ADGN-100 peptide. Peptides inhibiting the ACE2:SARS-CoV-2 Spike interactions with IC₅₀ below 1 μM, were further evaluated in complex with ADGN-106 peptide at a ⅕ peptide/ADGN molar ratio. Peptides were complexed with 5 or 10 molar excess of ADGN-106. The mean size and the polydispersity of peptide/ADGN-106 complexes were determined at 25° C. for 3 min per measurement and zeta potential was measured with Zetasizer 4 apparatus (Malvern Ltd). Data are shown in Table 8 for a mean of 3 separate experiments.

TABLE 8

| CODE SEQUENCE | Mean Size (nm) | PI |
|---|---|---|
| Seq12 (LNCOV-10) | 35.4 ± 5 | 0.21 |
| Seq15 (LNCOV-22) | 37.5 ± 5 | 0.23 |
| Seq16 (LNCOV-17) | 44.2 ± 7 | 0.28 |
| Seq17 (LNCOV-15) | 57 ± 4 | 0.17 |
| Seq19 (LNCOV-23) | 37.2 ± 5 | 0.24 |
| Seq20 (LNCOV-24) | 68.1 ± 5 | 0.25 |
| Seq21 (LNCOV-13) | 78.2 ± 7 | 0.3 |
| Seq22 (LNCOV-14) | 54.4 ± 8 | 0.24 |
| Seq27 (LNCOV-27) | 62.1 ± 10 | 0.21 |
| Seq28 (LNCOV-20) | 34.6 ± 7 | 0.18 |
| Seq32 (LNCOV-21) | 58.2 ± 9 | 0.17 |
| Seq33 (LNCOV-18) | 45.9 ± 10 | 0.24 |
| Seq35 (LNCOV-26) | 44 ± 10 | 0.22 |
| Seq37 (LNCOV-29) | 59.2 ± 14 | 0.24 |
| Seq38 (LNCOV-30) | 39 ± 10 | 0.25 |
| Seq39 (LNCOV-31) | 48.5 ± 5 | 0.29 |
| Seq40 (LNCOV-32) | 54.5 ± 14 | 0.31 |

As reported in Table 8 ADGN-106 forms stable complexes with the different peptides with a Mean diameters of about 35-50 nm and polydispersity index (PI) between 0.2-0.25 excepted for Seq23/Seq39/Seq40. In contrast no complex were detected in the presence of ADGN-106 or ADGN-100, for peptides corresponding to Seq1-11 and to Seq24-26, suggesting that structural organization and the size of the inhibitory peptides are important for complex formation.

### Example 6. Peptides Based Nanoparticle Inhibition of the ACE2:SARS-CoV-2 Spike Interactions

The potency of the different peptide inhibitor/ADGN-106 complexes to block the SARS-CoV-2 Spike: ACE2 interactions was evaluated in vitro using both the SARS-CoV-2 Spike: ACE2 and ACE2: SARS-CoV-2 Spike: Inhibitor Screening Assay Kit (DBS Bioscience). The results correspond to an average of 3 separate experiments and are reported in FIGS. 3B, FIG. 4B and Table 7.

As reported in FIGS. 3A-3B, FIGS. 4A-4B and Table 7, the association of the different peptides within ADGN-106 nanoparticles significantly improves their potency to block SARS-CoV-2 Spike: ACE2 interactions.

### A. ACE2: SARS-CoV-2 Spike Interaction

When complexed with ADGN-106 in the form of nanoparticles, SBP-1 control sequence prevents ACE2: spike inhibition with an IC₅₀ of 163.4±29 nM and SBP-1 covalently linked to ADGN-106 (Seq 32) with IC₅₀ of 24.5±2 nM.

The peptides Seqs 17, 19, 20, 21, 27, 28, 39, 40 when complexed with ADGN-106 in the form of nanoparticles inhibited SARS-CoV-2 Spike: ACE2 interaction with IC₅₀<10 nM. The best inhibition was obtained with Seqs 17, 20, 27, 28 with IC₅₀ value of 3.4±0.2, 3.7±0.5, 5.2±0.4, 1.8±0.8 and 2.7±0.7 nM, which are 40 to 6 folds more potent than SBP-1 peptide/ADGN-106 and SBP-1-ADGN-106/ADGN-106 complexes.

### B. SARS-CoV-2 Spike: ACE2 Interaction

When complexed with ADGN-106 in the form of nanoparticles, SBP-1 control sequence prevents ACE2: spike inhibition with an IC₅₀ of 87.5±15 nM and SBP-1 covalently linked to ADGN-106 (Seq 32) with IC₅₀ of 7.2±4 nM.

The peptides Seqs 17/27/28/33/39/40 when complexed with ADGN-106 in the form of nanoparticles inhibited ACE2: SARS-CoV-2 Spike interaction with IC₅₀<5 nM. The best inhibition was obtained with Seqs 17/27/28/33 with IC₅₀ value of 2.7±0.1, 1.1±0.5, 2.1±0.4 and 1.2±0.4 nM, which is 35 and 4 folds more potent than SBP-1 peptide/ADGN-106 and SBP-1-ADGN-106/ADGN-106 complexes.

TABLE 9
Results of nanoparticle forms of peptides

| | SARS-CoV-2 | ACE2:SARS- |
|---|---|---|
| | Spike:ACE2 | CoV-2 Spike |
| CODE SEQUENCE | IC₅₀ (nM) | IC₅₀ (nM) |
| Seq12 (LNCOV-10) | 45.4 | 268.5 |
| Seq17 (LNCOV-15) | 3.4 | 2.7 |
| Seq19 (LNCOV-23) | 7.8 | 132.5 |
| Seq20 (LNCOV-24) | 3.7 | 8.7 |
| Seq21 (LNCOV-13) | 5.2 | 11.2 |
| Seq22 (LNCOV-14) | 78.2 | 7.2 |
| Seq23 (SBP-1) | 163.4 | 87.5 |
| Seq27 (LNCOV-27) | 1.8 | 1.1 |
| Seq28 (LNCOV-20) | 2.7 | 2.1 |
| Seq32 (LNCOV-21) | 24.5 | 7.2 |
| Seq33 (LNCOV-18) | 27.6 | 1.2 |
| Seq35 (LNCOV-26) | 217.2 | 55.4 |
| Seq38 (LNCOV-30) | 91.4 | 49.7 |
| Seq39 (LNCOV-31) | 3.7 | 2.4 |
| Seq40 (LNCOV-32) | 2.1 | 3.4 |

Surprisingly, the results suggest that the association of the different peptides with ADGN-106 into nanoparticles significantly improves their potency to block SARS-CoV-2 Spike: ACE2 interactions. See Table 9. The formation of stable nanoparticles of ADGN-106 with the different peptides may increase locally the concentration of inhibitory domain resulting in improved efficiency.

In conclusion, the best candidates to block ACE2:SARS-CoV-2 Spike interactions correspond
A cyclic peptide corresponding to the β6-α5 loop covalently linked to ADGN-106 (Seq20 & Seq21)
A cyclic peptide corresponding to the β5-β6 loop covalently linked to ADGN-106 (Seq17)
A peptide derived from α1 and α2 helices of ACE2 coupled or not to ADGN-106 (Seq27, 28, 39, 40).
A peptide derived from α1 helix of ACE2 covalently linked to ADGN-106 (Seq33)

### Example 7. Screening of Peptides Inhibitor of SARS-CoV 2 Virus Infection

The impact of the different peptides (not in the nanoparticle form) on SARS-CoV2 virus infection was evaluated on Vero E6 cells. Cells were plated in 96 wells one day prior to infection. Peptide solutions were made in DMSO/water (2%) and diluted from stock of 5 mM. The screening was performed using a single concentration of peptide of 0.5 μM. Peptides were mixed with SARS-CoV-2 (MOI 0.01) for 30 min and then added to monolayer Vero-E6 cells. 72 hours post infection, the plates were fixed, stained and analyzed. In parallel uninfected cells were used to monitor cytotoxicity of the different peptides alone using CellTiter-Glo assays (Promega).

Results are reported in Table 10.

TABLE 10

LNCOV

| | Residual Infection | Cell Viability |
|---|---|---|
| CODE SEQUENCE | (%) | (%) |
| Seq15 (LNCOV-22) | 100 | 95 |
| Seq17 (LNCOV-15) | 20 | 85 |
| Seq19 (LNCOV-23) | 90 | 87 |
| Seq20 (LNCOV-24) | 65 | 91 |
| Seq21 (LNCOV-13) | 79 | 78 |
| Seq22 (LNCOV-14) | 40 | 87 |
| Seq28 (LNCOV-20) | 10 | 78 |
| Seq32 (LNCOV-21) | 70 | 81 |
| Seq33 (LNCOV-18) | 15 | 94 |
| Seq34 (LNCOV-25) | 100 | 98 |
| Seq38 (LNCOV-30) | 80 | 68 |
| ADGN-106 | 80 | 84 |

As reported in Table 10, at a 0.5 μM concentration, peptides Seq17, 20, 22, 28 and 33 exhibit inhibition of viral infection. Seq17, 28 and 33 reduce by more than 80% of virus infection, and Seq22 by 60%, with a very low cellular toxicity. Therefore, the peptides Seq17, 22, 28 and 33 were further evaluated on SARS-CoV-2 virus infectivity and replication to obtain full IC50 information for inhibition of infection.

Cells were plated in 96 wells one day prior to infection. Peptide solutions were made in DMSO/water (2%) and diluted from stock of 5 mM. Peptides with different dilution concentrations were either directly added to monolayer Vero-E6 cells immediately prior to virus infection or mixed with SARS-CoV-2 for 30 min and then added to monolayer Vero-E6 cells. ADGN-106, hydroxy-chloroquine and buffer were used as control and all treatments were performed in triplicate. The cellular toxicity of the different peptides was analyzed on Vero-6, H1299 and HEPG2, cells using CellTiter-Glo assays (Promega).

Results are reported in Table 11.

TABLE 11

| Compounds | P-IC50 (nM) | D-IC50 (nM) | TD50 (μM) |
|---|---|---|---|
| Seq 22 (LNCOV-14) | 145.2 ± 25 | 124.5 ± 17 | 358 ± 27 |
| Seq 17 (LNCOV-15) | 57.2 ± 12 | 21.5 ± 12 | 340 ± 28 |
| Seq 33 (LNCOV-18) | 12.2 ± 5 | 21.7 ± 8 | 510 ± 31 |
| Seq 28 (LNCOV-20) | 17.7 ± 9 | 34.5 ± 11 | 290 ± 51 |
| ADGN-106 | 6800 ± 1200 | 5450 ± 956 | 356 ± 64 |
| Hydroxy chloroquine | 4700 ± 523 | 4400 ± 215 | 190 ± 28 |

*P-IC₅₀ values were obtained when SARS-CoV-2/peptide were pre-incubated prior infection.
*D-IC₅₀ value were obtained when peptide were directly applied to Vero 6 cells.

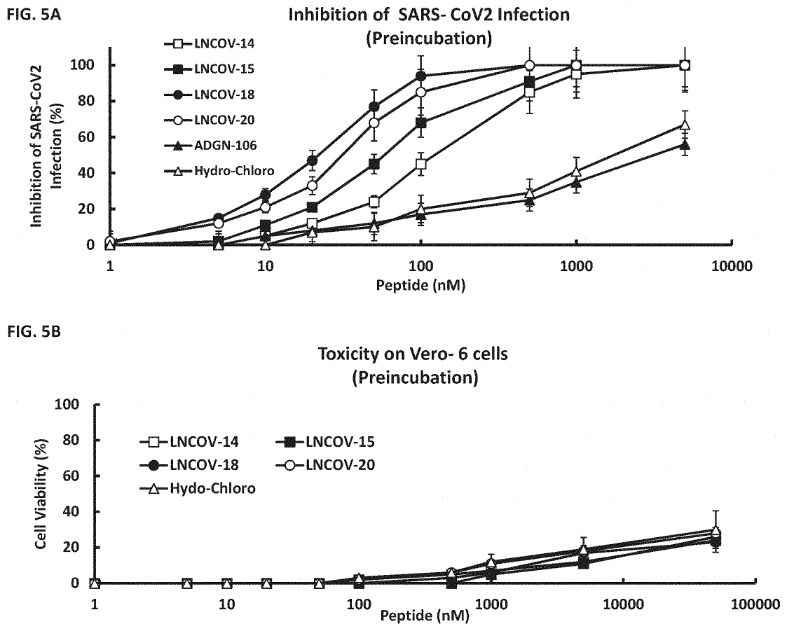

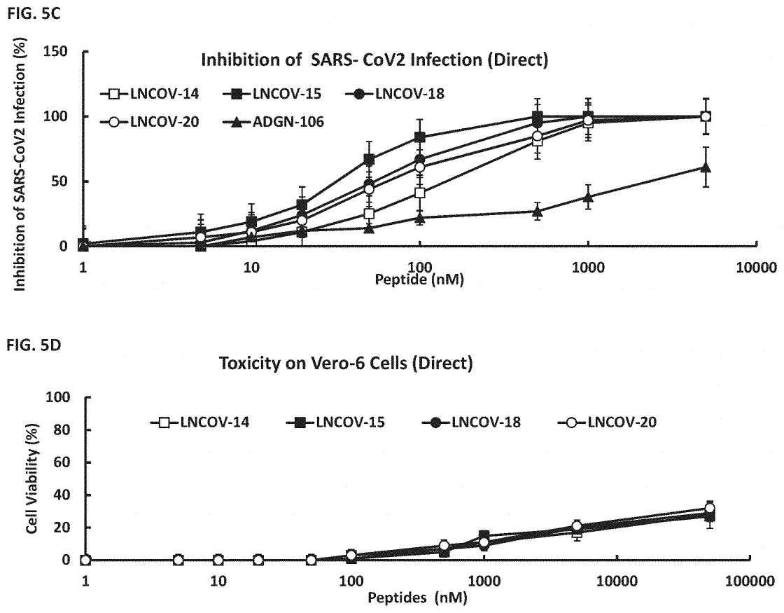

As reported in FIGS. 5A, 5C and Table 11, all four peptides showed a potent inhibitory activity against SARS-CoV2 virus infections with IC₅₀ in the nanomolar range. Seq 17 and Seq 22 exhibited an IC₅₀ of 21.5±12 nM and 124.5±14 nM respectively. Seq 28 and Seq 33 exhibit an IC₅₀ of 34.5±11 nM and 21.7±8 nM respectively. Pre-incubation with SARS CoV-2 prior infection increased by a factor of 2 the potency of Seq 28 and Seq 33, which is in agreement with the fact that these peptides interacted with the Spike protein and therefore will interact directly will the virus. In contrast, pre-incubation does not affected Seq 22 potency and reduced by 2-fold Seq 17 anti-viral activity. ADGN-106 alone showed a poor antiviral activity with an IC₅₀ between 5.5-6.8 μM.

All the peptides exhibits a relatively low cytotoxicity on uninfected cells with TD₅₀ values ranging between 300 to 500 μM. See FIGS. 5B and 5D.

Seq 17/28/33 constitute very potent inhibitors of SARS-CoV-2 infection providing 250-fold stronger anti-SARS-CoV-2 activity than hydroxychloroquine sulfate (IC₅₀ 4.4±0.215 μM).

### Example 8. Selection of siRNA Targeting SARS-CoV-2 Nucleocapsid

Ten siRNAs targeting SARS-CoV-2 Nucleocapsid gene were selected (Table 12)

TABLE 12

| | | | IC50 |
|---|---|---|---|
| siRNA | Positions | Sequences | (nM) |
| DIVC-1 | 400 | GCAACTGAGGGAGCCTTGAAT | >500 |
| | | (SEQ ID NO: 161) | |
| DIVC-2 | 862 | GACCAGGAACTAATCAGACAA | >500 |
| | | (SEQ ID NO: 162) | |
| DIVC-3 | 206 | GACAAGGCGTTCCAATTAACA | 11.4 |
| | | (SEQ ID NO: 163) | |
| DIVC-4 | 359 | GACTTCCCTATGGTGCTAACA | 254 |
| | | (SEQ ID NO: 164) | |
| DIVC-5 | 1122 | GGCTGATGAAACTCAAGCCTT | >500 |
| | | (SEQ ID NO: 165) | |
| DIVC-6 | 727 | GGCCAAACTGTCACTAAGAAA | 8.7 |
| | | (SEQ ID NO: 166) | |
| DIVC-7 | 824 | GCAGACGTGGTCCAGAACAAA | 147 |
| | | (SEQ ID NO: 167) | |
| DIVC-8 | 544 | GCCTCTTCTCGTTCCTCATCA | 27.3 |
| | | (SEQ ID NO: 168) | |
| DIVC-9 | 1063 | AAGCATATTGACGCATACAAA | 394.2 |
| | | (SEQ ID NO: 169) | |
| DIVC-10 | 1221 | GCAACAATCCATGAGCAGTGC | 28.4 |
| | | (SEQ ID NO: 170) | |

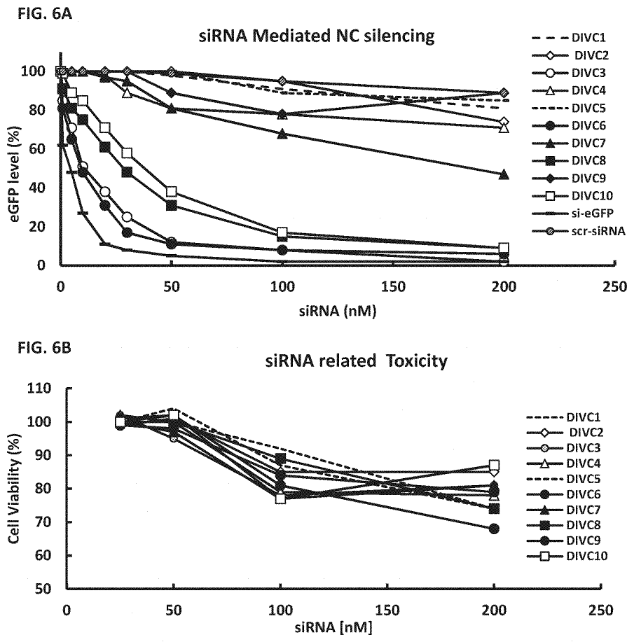

The siRNA were evaluated on H1299 lung epithelial cells expressing eGFP-SARS-CoV-2 nucleocapside. H1299 epithelial cells were transfected with pcDNA3.1(+)-N-eGFP-NP plasmid encoding for SARS-COV-2 nucleocapside-tagged with eGFP (Genscript Ref MC-0101137). Cells were then treated with siRNAs (1 nM to 200 nM) complexed with ADGN-100 at molar ratio 1/20. A siRNA targeting eGFP and scr-siRNA were used as positive and negative controls, respectively. The level of Nucleocapside-eGFP protein was evaluated by Elisa (Abcam) 48 hr post transfection and toxicity was determined using CellTiter Glow kits on GlowMax (Promega). Results are reported in FIGS. 6A-6B and Table 12.

As reported in FIG. 6A, siRNA targeting eGFP induced eGFP silencing with an IC₅₀ of 4.5±2 nM. 4 siRNAs (DIVC-6/DIVC-3/DIVC-8/DIVC-10) induced NC silencing at low nanomolar concentrations. Best silencing response was obtained with DIVC-6 and DIVC-3 with IC₅₀ of 8.7±2 nM and 11.4±5 nM, respectively. No significant toxicity associated to the siRNA was observed. See FIG. 6B.

### Example 9. Selection of siRNA Targeting SARS-CoV-2 ORF 3A Gene

Five different siRNAs targeting SARS-CoV-2 ORF 3A gene were selected (Table 13)

TABLE 13

| siRNA | Positions | Sequences | IC₅₀ (nM) |
|---|---|---|---|
| DIVC-31 | 52 | GGTGAAATCAAGGATGCTACT | 18.4 ± 2 |
| | | (SEQ ID NO: 171) | |
| DIVC-32 | 485 | GTGTAACTTCTTCAATTGTCA | 95.1 ± 7 |
| | | (SEQ ID NO: 172) | |
| DIVC-33 | 527 | CAAGTCCTATTTCTGAACATG | 57.2 ± 12 |
| | | (SEQ ID NO: 173) | |
| DIVC-34 | 676 | GAACATGTTACCTTCTTCATC | 12.3 ± 1 |
| | | (SEQ ID NO: 174) | |
| DIVC-35 | 366 | TGCAGAGTATAAACTTTGTAA | 125 ± 11 |
| | | (SEQ ID NO: 175) | |

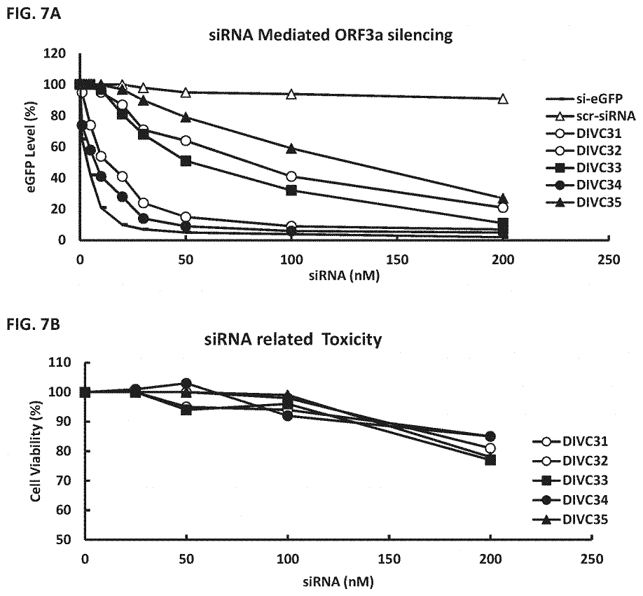

The siRNA were evaluated on H1299 lung epithelial cells expressing eGFP-SARS-CoV-2 ORF3A gene. H1299 epithelial cells were transfected with pcDNA3.1(+)-N-eGFP-ORF3a plasmid encoding for SARS-COV-2 ORF3a-tagged with eGFP (Genscript Ref MC-0101137). Cells were then treated with siRNAs (1 nM to 200 nM) complexed with ADGN-100 at molar ratio 1/20. A siRNA targeting eGFP and scr-siRNA were used as positive and negative controls, respectively. The level of ORF3a-eGFP protein was evaluated by Elisa (Abcam) 48 hr post transfection and toxicity was determined using CellTiter Glow kits on GlowMax (Promega). Results are reported in FIGS. 7A and 7B and Table 13.

As reported in FIG. 7A, siRNA targeting eGFP induced eGFP silencing with an IC₅₀ of 4.5±2 nM.

DIVC-31 and DIVC-34 siRNAs induced ORF 3a protein silencing at low nanomolar concentrations with IC₅₀ of 18.4±2 nM and 12.3±1 nM, respectively. No significant toxicity associated to the siRNA was observed.

### Example 10. Selection of siRNA Targeting SARS-CoV-2 ORF 8 Gene

Five siRNAs targeting SARS-CoV-2 ORF 8 gene were selected (Table 14)

TABLE 14

| siRNA | Positions | Sequences | IC₅₀ (nM) |
|---|---|---|---|
| DIVC-81 | 97 | GTTGATGACCCGTGTCCTATT | >200 |
| | | (SEQ ID NO: 176) | |
| DIVC-82 | 103 | GACCCGTGTCCTATTCACTTC | 21.7 ± 7 |
| | | (SEQ ID NO: 177) | |
| DIVC-83 | 184 | GTGGATGAGGCTGGTTCTAAA | 15.4 ± 3 |
| | | (SEQ ID NO: 178) | |
| DIVC-84 | 300 | GCGTTGTTCGTTCTATGAAGA | 112.8 ± 15 |
| | | (SEQ ID NO: 179) | |
| DIVC-85 | 110 | GTCCTATTCACTTCTATTCTA | 10.8 ± 2 |
| | | (SEQ ID NO: 180) | |

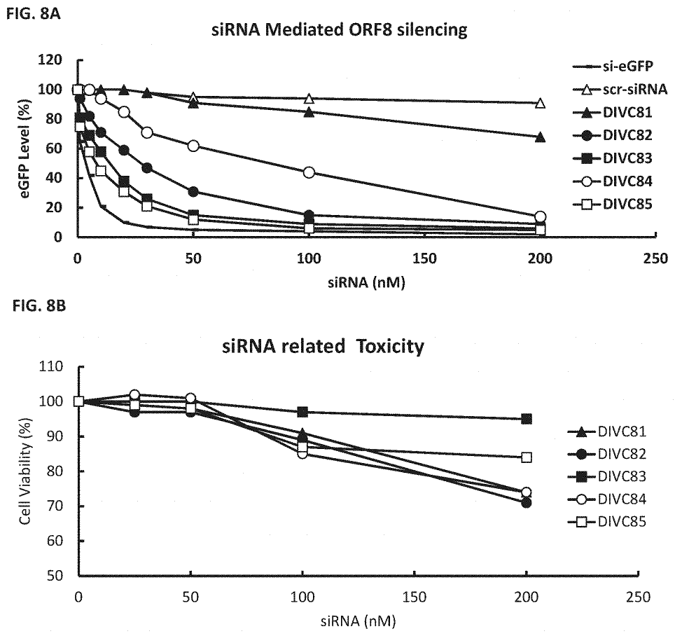

The siRNA were evaluated on H1299 lung epithelial cells expressing eGFP-SARS-CoV-2 ORF8 gene. H1299 epithelial cells were transfected with pcDNA3.1(+)-N-eGFP-ORF8 plasmid encoding for SARS-COV-2 ORF8-tagged with eGFP (Genscript Ref MC-0101137). Cells were then treated with siRNAs (1 nM to 200 nM) complexed with ADGN-100 at molar ratio 1/20. A siRNA targeting eGFP and scr-siRNA were used as positive and negative controls, respectively. The level of ORF8-eGFP protein was evaluated by Elisa (Abcam) 48 hr post transfection and toxicity was determined using CellTiter Glow kits on GlowMax (Promega). Results are reported in FIGS. 8A-8B and Table 14.

As reported in FIG. 8A, DIVC-82, DIVC-83 and DIVC-85 siRNAs induced ORF 8a protein silencing with IC₅₀ of 21.7±7 nM, 15.4±3 nM and 10.8±2 nM, respectively. No significant toxicity associated to the siRNA was observed.

### Example 11. Screening of siRNA of SARS-CoV-2 Virus Infection

The impact of the siRNA on SARS-CoV2 virus infection was evaluated on Vero E6 cells. Cells were plated in 96 wells one day prior to infection. siRNAs (DIVC-3, DIVC-6, DIVC-8, DIVC-34 and DIVC-85) were complexed with ADGN-100 at molar ratio 1/20. siRNA/ADGN-100 complexes with different dilution concentrations were directly added to monolayer Vero-E6 cells immediately prior to virus infection. ADGN-100, and buffer were used as control and all treatments were performed in triplicate. The cellular toxicity of the different siRNA/ADGN-100 complexes was analyzed on Vero-6 cells using CellTiter-Glo assays (Promega).

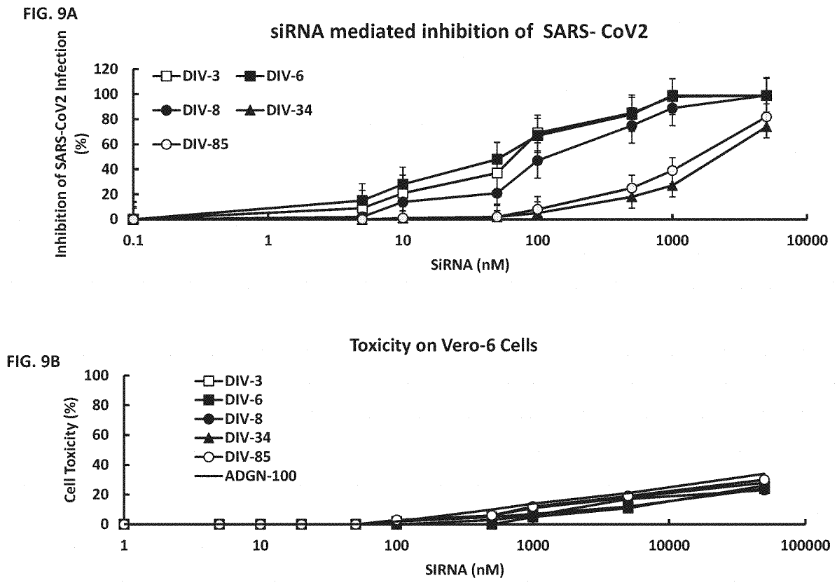

Results are reported in FIGS. 9A-9B and Table 15.

TABLE 15

| SIRNA | IC50 (nM) | TD50 (μM) |
|---|---|---|
| DIVC-3 | 84 + 14 | 452 + 20 |
| DIVC-6 | 68 + 12 | 652 + 24 |
| DIVC-8 | 117 + 8 | 548 + 25 |
| DIVC-34 | 1450 + 11 | 589 + 17 |
| DIVC-85 | 1221 + 18 | 248 + 35 |
| AGDN-100 | 7500 + 523 | 368 + 18 |

As reported in FIG. 9 and Table 15, siRNA targeting SARS-CoV-2 Nucleocapsid gene (DIVC-3, DIVC-6 and DIVC-8) showed a potent inhibitory activity against SARS-CoV2 virus infections with IC₅₀ in the nanomolar range DIVC-3 and DIVC-6 exhibit an IC₅₀ of 84±14 nM and 68±12 nM respectively. In contrast, siRNA targeting SARS-CoV-2 ORF8 (DIVC-85) or ORF3A (DIVC-34) showed a moderate anti-viral activity with IC₅₀ around 1 μM. ADGN-100 alone showed a poor antiviral activity with an IC₅₀ of 7.5 μM. The data suggesting that nucleocapsid is essential for the virus production, in contrast to ORF3A and ORF8 proteins.

All the siRNA/ADGN-100 complexes exhibit a relatively low cytotoxicity on uninfected cells with TD₅₀ values ranging between 300 to 600 μM.

DIVC-6 siRNA targeting SARS-CoV-2 Nucleocapsid gene constitute very potent inhibitor of SARS-CoV-2 infection providing 64-fold stronger anti-SARS-CoV-2 activity than hydroxychloroquine sulfate (IC₅₀ 4.4±0.215 μM).

### Example 12. Impact of siRNA and Peptide Inhibitor Combination on SARS-CoV 2 Virus Infection

We demonstrated that DIVC-6 siRNA targeting SARS-CoV-2 Nucleocapsid gene, Seq 17 (LNCOV-15) and 33 (LNCOV-18) constitute very potent inhibitors of SARS-CoV-2 infection. We next evaluated the impact of combining siRNA targeting SARS-CoV-2 Nucleocapsid gene with peptide inhibitors of the SARS-Cov2: ACE2 receptor on viral replication.

The impact of the siRNA/peptide inhibitor association on SARS-CoV2 virus infection was evaluated on Vero E6 cells. Cells were plated in 96 wells one day prior to infection. DIVC-6 siRNA was complexed with ADGN-100 at molar ratio 1/20. siRNA/ADGN-100 complexes and Seq17 or Seq33 peptides with different dilution concentrations were directly added to monolayer Vero-E6 cells immediately prior to virus infection. ADGN-100, and buffer were used as control and all treatments were performed in triplicate.

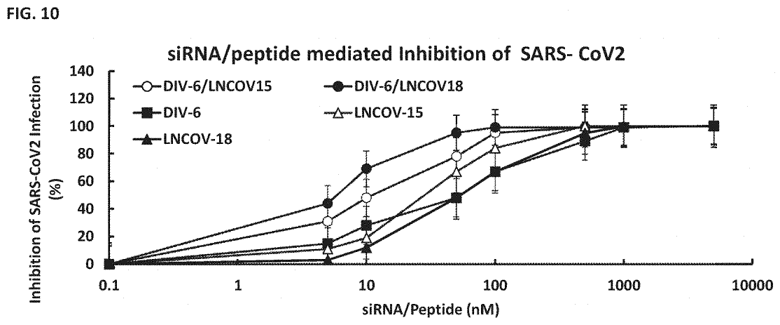

Results are reported in FIG. 10 and Table 16.

TABLE 16

| Treatments | IC50 (nM) |
|---|---|
| DIVC-6/ADGN-100 | 68 ± 12 |
| LNCOV-15 | 27.5 ± 11 |
| LNCOV-18 | 22.9 ± 8 |
| DIVC-6/LNCOV-15 | 8.4 ± 8 |
| DIV6/LNCOV-18 | 11.2 ± 4 |

As reported in FIG. 10 and Table 16, combining LNCOV-15 or LNCOV-18 peptides with DIVC-3 siRNA targeting SARS-CoV-2 Nucleocapsid gene improved by a 2-3 folds the potency of the free peptide and by 6 folds the potency of the DIVC-6 siRNA, with IC₅₀ of 8.4±8 nM and 11.2±4 nM for DIVC-6/LNCOV-15 and DIVC-6/LNCOV-18 combination, respectively. The results demonstrated that combining siRNA together with inhibitor peptides allowed the targeting of two different steps in the virus replication including cell entry and replication.

### Example 13. LNCOV-15 and LNCOV-18 Promotes siRNA Delivery in Cultured Cells

The potency of LNCOV-15 and LNCOV-18 to promote siRNA delivery in cultured cells have been evaluated using siRNA targeting SARS-CoV-2 nucleocapside.

The siRNA DIVC-6 was evaluated on H1299 lung epithelial cells expressing eGFP-SARS-CoV-2 nucleocapside. H1299 epithelial cells were transfected with pcDNA3.1(+)-N-eGFP-NP plasmid encoding for SARS-COV-2 nucleocapside-tagged with eGFP (Genscript Ref MC-0101137). Cells were then treated with siRNAs (1 nM to 200 nM) complexed with ADGN-100, LNCOV-15 and LNCOV-18 at molar ratio 1/20. A scr-siRNA was used as positive and negative controls, respectively. The level of Nucleocapside-eGFP protein was evaluated by Elisa (Abcam) 48 hr post transfection and toxicity was determined using CellTiter Glow kits on GlowMax (Promega). Results are reported in FIG. 11A.

As reported in FIG. 11A, LNCOV-15 and LNCOV-18 promote siRNA delivery in cultured cells, resulting in the silencing of NC silencing at low nanomolar concentrations. A silencing response associated to DIVC-6 siRNA was obtained with IC₅₀ of 11±4 nM and 8.7±2 nM when complexed with LNCOV-15 and LNCOV-18 respectively. The results are similar to the one obtained with ADGN-100 suggesting that LNCOV peptides are potent siRNA delivery vector and can be used for both siRNA delivery and to block virus cell entry.

### Example 14: Inhibition of the ACE2:SARS-CoV-2 Spike Variants Interaction Using Peptide Inhibitors

Several new variants of SARS-CoV-2 virus have emerged in recent months. The U.K. variant B.1.1.7, the Brazil variant P.1, the South Africa variant B.1.351, and the most recent Indian variant B.1.617 are particular concerning because of their high prevalence. A subset of the mutations identified in the RBD domain of the spike protein occurs in more than one strain, and have been reported to increase transmissibility, infectivity and may increase their immune evasion potential.

The potency of the lead peptides Seq17 (LN-COV-15), Seq28 (LN-COV-20) and Seq33 (LN-COV-18) to block the interaction between ACE2 and SARS-CoV-2 Spike variants was evaluated in vitro using the ACE2:SARS-CoV-2 Spike Inhibitor Screening Assay Kit (DBS Bioscience). Peptide inhibition was evaluated on the five different SARS-COv-2 Spike protein variants harboring the major mutations including Alpha (B1.1.7/United Kingdom), Beta (B1.351/South Africa), Gamma (P1/Brazil), Delta (B1 617 2/India) and Epsilon (B1429) variants (Table 17). RBD and Spike protein variants were obtained from Sino Biological (US).

TABLE 17

| Variants | Lineage | Spike mutations |
|---|---|---|
| Alpha (UK) | B 1 1 7 | H69-V70 deletion, N501Y, |
| | | D614G |
| Beta (South Africa) | B 1 53 5 1 | K417N, E484K, N501Y, |
| | | D614G |
| Gamma (Brazil) | P1 | L18F, T20N, P26S, D138Y, |
| | | R190S, K417T, E484K, |
| | | N501Y, D614G, H655Y |
| Delta (India) | B 1.6.1.7.2 | T19R, G142D, E156G, |
| | | L452R, T478K, D614G, |
| | | P681R |
| Epsilon (USA) | B.1.429/B.1.427 | S13I, W152C, L452R, |
| | | D614G |

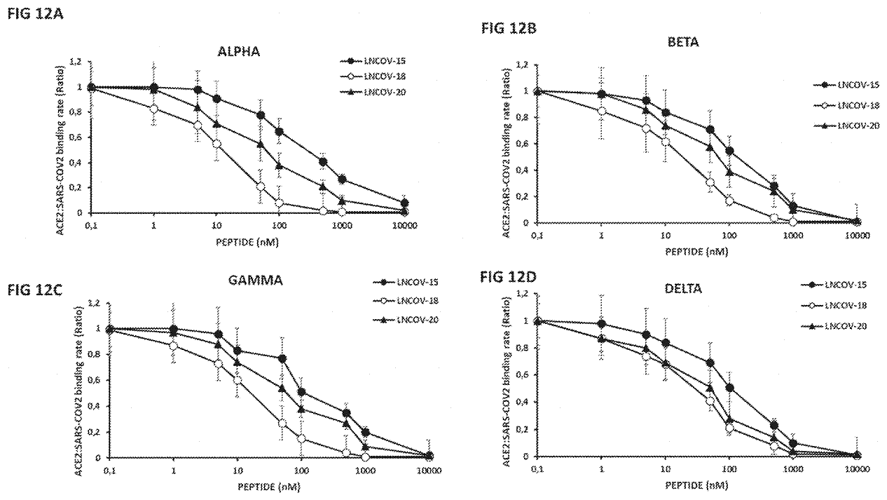

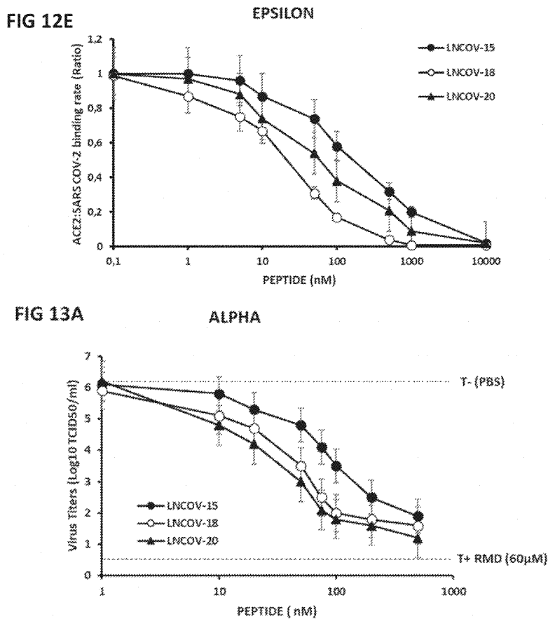

Peptide evaluation was performed in 96-well format. The ACE2 protein was attached to a nickel-coated 96-well plate, then, incubated with peptide inhibitor solutions at room temperature for 30 min with slow shaking. Finally the different SARS-CoV-2 Spike-Fc was added to the ACE2 on the plate in the presence of peptide inhibitor solutions and incubate at room temperature for one hour with slow shaking. The plates were treated with Anti-Fc-HRP followed by addition of an HRP substrate to produce chemiluminescence and analyzed using a chemiluminescence reader. The results correspond to an average of 3 separate experiments and are reported in FIGS. 12A-12E and Table 18.

TABLE 18

| | SARS- | Alpha | Beta | Gamma | Delta | Epsilon |
|---|---|---|---|---|---|---|
| Peptides | COV2 | (B117) | (B1312) | (P1) | (B1617) | (B1429) |
| Seq 17 | 78.2 ± 14 nM | 135.5 ± 21 nM | 75.2 ± 21 nM | 87.2 ± 20 nM | 48.2 ± 11 nM | 94.7 ± 17 nM |
| (LNCOV-15) | | | | | | |
| Seq28 | 38.6 ± 11 nM | 24.6 ± 11 nM | 31.5 ± 05 nM | 41.7 ± 11 nM | 29.2 ± 11 nM | 28.8 ± 07 nM |
| (LNCOV-20) | | | | | | |
| Seq33 | 18.4 ± 11 nM | 17.8 ± 09 nM | 28.8 ± 10 nM | 20.2 ± 05 nM | 25.7 ± 08 nM | 31.4 ± 09 nM |
| (LNCOV-18) | | | | | | |

As reported in FIGS. 12A-12E, the lead peptides block the interaction between ACE2 and different SARS-CoV-2 Spike variants. In all cases, IC50 values are similar to those obtained with the SARS-COV2 original strain.

The Seq28 and Seq33 peptides are the most efficient with IC50 below 50 nM whatever the Spike variant used. The Seq28 and Seq33 peptides directly targeted Spike protein and competed for the interface onto ACE2. This interface ACE2/Spike involves residues N501, E484, L452 and K417, which are mutated in the different variants with better affinity for ACE2.

The mutation L452R (Variant Delta and Epsilon) have been associated with immune escape and greater transmissibility. This mutation is directly targeted by Seq-28 and Seq-33.

Mutations on E484 residue have been reported to increase infectivity of the virus and strengthens interaction with ACE2. This mutation E484K (variant P Gamma and Beta) and E484Q (variant Delta) is directly targeted by Seq17 which competed for the same spike/ACE2 interface.

### Example 15: Evaluation of Peptide Inhibitors on SARS-CoV 2 Variant Virus Infection

The potency of the lead peptides Seq17 (LN-COV-15), Seq28 (LN-COV-20) and Seq33 (LN-COV-18) to block SARS-CoV-2 variant virus infectivity and replication was further evaluated.

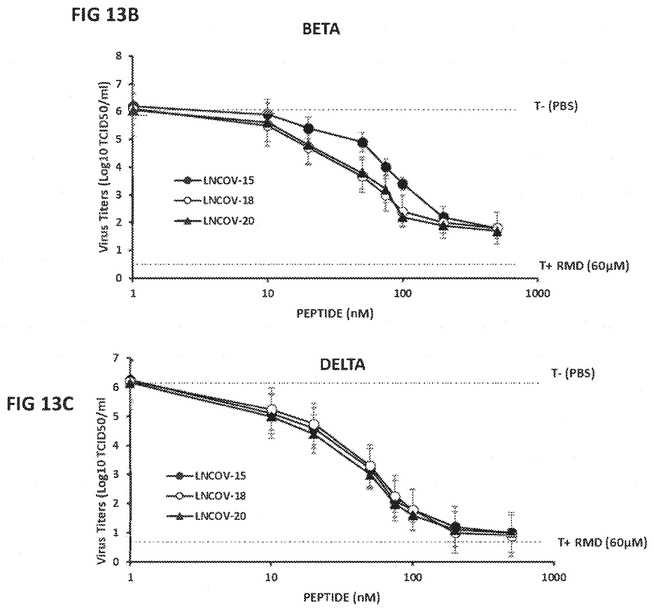

The antiviral assay was performed on Vero E6 cells in DMEM high glucose medium (D6429; Sigma Aldrich) supplemented with 2% FBS (Eurobio-Scientific) and 1% Penicillin-Streptomycin solution (P0781; Sigma Aldrich). Cells were infected in triplicate with SARS-CoV-2 variants (Alpha/Beta/Delta) at MOI 0.001 by incubation for 1 hour in mediums containing either Seq17, Seq28 or Seq33 peptides (concentration ranging from 10 nM to 1 μM) or 6 μM of remdesivir (positive control RMD), or no antiviral molecule (negative control; “T-”). Peptide solutions were made in DMSO/water (2%) and diluted from stock of 5 mM. Inoculums were removed 1-hour post-infection and cells were washed with PBS 1×before new mediums with previously indicated concentrations were added. Supernatants were harvested 24 hours post-infection and viral titers were determined by the TCID50 method on Vero E6 cells and calculated by the Spearman & Kärber algorithm. Results are reported in FIGS. 13A-13C and Table 19.

TABLE 19

| | | Alpha | Beta | Delta |
|---|---|---|---|---|
| Peptides | SARS-COV2 | (B117) | (B1312) | (B1617) |
| Seq17 (LNCOV-15) | 31.5 ± 12 nM | 125.2 ± 24 nM | 118.2 ± 15 nM | 25.7 ± 10 nM |
| Seq28(LNCOV-20) | 34.5 ± 11 nM | 28.4 ± 09 nM | 57.5 ± 11 nM | 18.2 ± 11 nM |
| Seq33 (LNCOV-18) | 21.7 ± 8 nM | 48.2 ± 15 nM | 47.2 ± 14 nM | 32.2 ± 09 nM |

As reported in FIGS. 13A-13C and Table 19, all three peptides showed a potent inhibitory activity against all SARS-CoV2 variant viruses and achieved viral inhibitions greater than 99.99% at the maximum concentration tested (500 nM). For Alpha/Beta and Delta variants, IC50 values obtained with Seq28 and Seq33 are similar to those obtained with the SARS-COV2 original strain. Seq17 is 3 to 4-fold less efficiency on Alpha and Beta variants, respectively.

Seq 28 and Seq 33 constitute very potent inhibitors of all SARS-CoV-2 variant infection providing 200-fold stronger anti-SARS-CoV-2 activity than Remdesivir.

### Example 16. Impact of siRNA and Peptide Inhibitor Combination on SARS-CoV 2 Virus Variant Infection

We demonstrated that DIVC-6 siRNA targeting SARS-CoV-2 Nucleocapsid gene, Seq17 (LNCOV-15), seq 28 (LNCOV-20) and seq 33 (LNCOV-18) constitute very potent inhibitors of SARS-CoV-2 infection. We have evaluated the impact of combining siRNA targeting SARS-CoV-2 Nucleocapsid gene with peptide inhibitors of the SARS-Cov2: ACE2 receptor on the replication of SARS-Cov-2 Alpha, Beta and Delta variants.

The impact of the siRNA/peptide inhibitor association on SARS-CoV2 virus infection was evaluated on Vero E6 cells. Cells were plated in 96 wells one day prior to infection. DIVC-6 siRNA was complexed with ADGN-100, Seq-17, Seq-28 or Seq-33 at molar ratio 1/20. siRNA/peptide complexes and Seq17, Seq-28 and Seq33 peptides with different dilution concentrations were directly added to monolayer Vero-E6 cells immediately prior to virus infection. 6 μM of remdesivir (positive control RMD), or no antiviral molecule (negative control; “T-”) were used as control and all treatments were performed in triplicate.

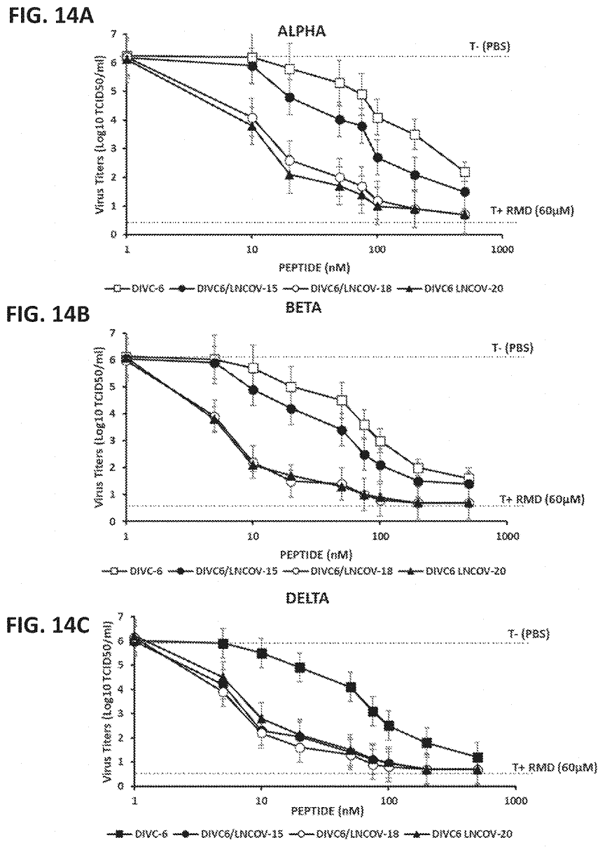

Results are reported in FIGS. 14A-14C and Table 19.

TABLE 19

| | SARS- | Alpha | Beta | Delta |
|---|---|---|---|---|
| | COV2 | (B117) | (B1312) | (B1617) |
| Compounds | IC₅₀ (nM) | IC₅₀ (nM) | IC₅₀ (nM) | IC₅₀ (nM) |
| DIVC-6 | 68 + 12 | 84.5 + 10 | 58.5 + 11 | 91.3 + 15 |
| Seq17 (LNCOV- | 31.5 ± 12 | 125.2 ± 24 | 118.2 ± 15 | 25.7 ± 10 |
| 15) | | | | |
| DIVC-6/seq17 | 11.4 ± 4 | 21.4 ± 5 | 27.5 ± 10 | 8.7 ± 5 |
| Seq28(LNCOV- | 34.5 ± 10 | 28.4 ± 09 | 57.5 ± 11 | 18.2 ± 11 |
| 20) | | | | |
| DIVC-6/Seq-28 | 16.8 ± 9 | 6.5 ± 4 | 5.9 ± 3 | 9.8 ± 5 |
| Seq33 (LNCOV- | 21.7 ± 8 | 48.2 ± 15 | 47.2 ± 14 | 32.2 ± 09 |
| 18) | | | | |
| DIVC-6/Seq-33 | 17.5 ± 4 | 11.8 ± 7 | 5.7 ± 1 | 6.9 ± 4 |

As reported in FIG. 14A-14C, DIVC-6 siRNA showed a potent inhibitory activity against all SARS-CoV2 variant viruses. This is not surprising as the sequence targeted by DIVC-6 is located at the N-terminal part of the CTD domain of the nucleocapsid which have been associated to a poor rate of mutation and is not affected in the different variants tested.

As reported for SARS-COV-2 original strain, combining Seq17, Seq28 and Seq33 peptides with DIVC-6 siRNA targeting SARS-CoV-2 Nucleocapsid gene improved by 2-3 folds the potency of the free peptide and by 6-10 folds the potency of the DIVC-6 siRNA.

The results demonstrated that combining siRNA together with inhibitor peptides allowed the targeting of two different steps in the virus replication including cell entry and replication.

### Example 17. Lung Administration of Peptide and Peptide/siRNA Complex Inhibitors of SARS-Cov 2

DIVC-6 siRNA targeting SARS-CoV-2 Nucleocapsid gene, Seq17 (LNCOV-15) and seq 33 (LNCOV-18) constitute very potent inhibitors of SARS-CoV-2 infection. As SARS-CoV-2 infects lung tissue through breathing, we aim to use the same route to deliver peptide inhibitors and peptide/siRNA complexes.

Considering that for the lung treatment, it is important to obtain significant tissue penetration and to get into the deep lung, we have evaluated lung delivery and distribution, using intratracheal instillation, of Seq17 (LNCOV-15), seq 33 (LNCOV-18), seq17/DIVC-6 and Seq33/DIVC-6 complexes. To monitor Seq-33 and Seq-17 biodistribution, the peptides were labelled at the N-terminus with Cy5.5 dye. To monitor DIVC-6 siRNA biodistribution, the siRNA was labelled with Cy-5.5.

In vivo lung biodistribution study was performed in healthy 4 weeks old male C57BL/6J mice. Single dose of peptides or peptide/siRNA complexes were administrated via intratracheal instillation (200 μg) (Table 20). The fluorescence was evaluated by non-invasive in vivo whole-body fluorescence imaging at T0, 1 h, 2 h, 6 h, 24 h, 48 h and 72 hr post injection. Fluorescence images were acquired with the IVIS kinetic system (PerkinElmer) (ex: 640±17 nm; em: 680±10 nm) and semi-quantitative data were obtained from the fluorescence images by using the Living image software (Caliper 2D).

TABLE 20

| | Mouse | Dosage/ | | Mice/ |
|---|---|---|---|---|
| Group | Group | Mouse | Volume | group |
| 1 | Control | 200 μg | 25 μl | 6 |
| | | (10 mg/Kg) | | |
| 2 | Seq-17 | 200 μg | 25 μl | 6 |
| | | (10 mg/kg) | | |
| 3 | Seq-33 | 200 μg | 25 μl | 6 |
| | | (10 mg/kg) | | |
| 4 | Seq-33/ | 200 μg | 25 μl | 6 |
| | DIVC-6 | (10 mg/Kg) | | |
| 5 | Seq-17/ | 200 μg | 25 μl | 6 |
| | DIVC-6 | (10 mg/Kg) | | |

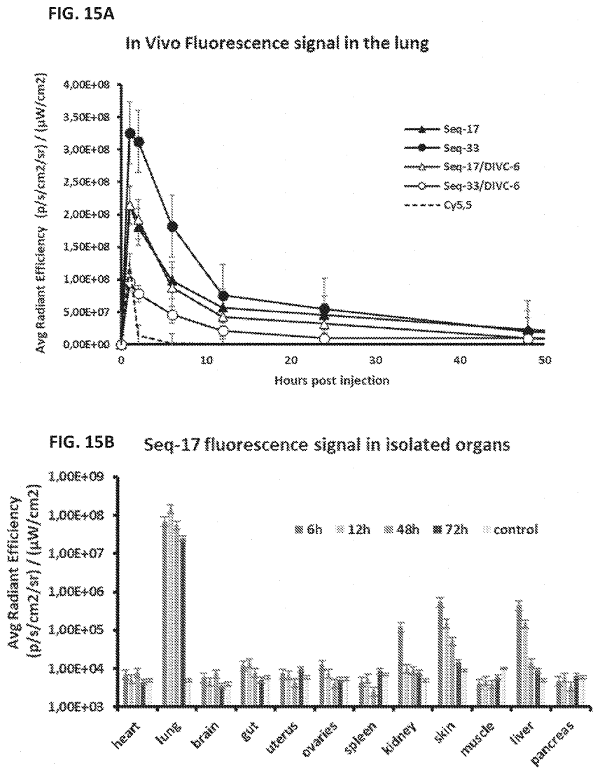

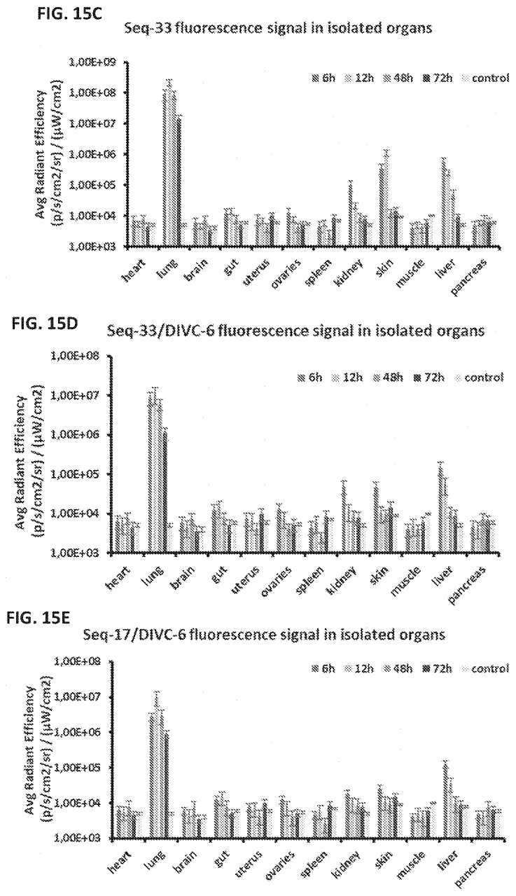

As reported in FIGS. 15A-15E, after intratracheal instillation in mice, all compounds provide a very strong signal in the lung which remains high until 72 hr post administration. In contrast, no signal was detected for free Cy 5.5 dye after 4 hr. All compounds are rapidly eliminated by the bladder and liver at 24 h and 48 h post injection. At 72 hr, only the lung display a signal detectable in vivo.

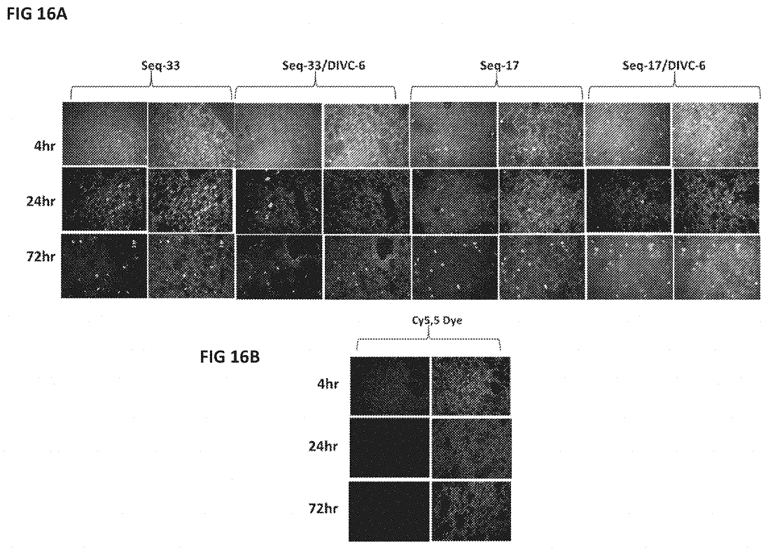

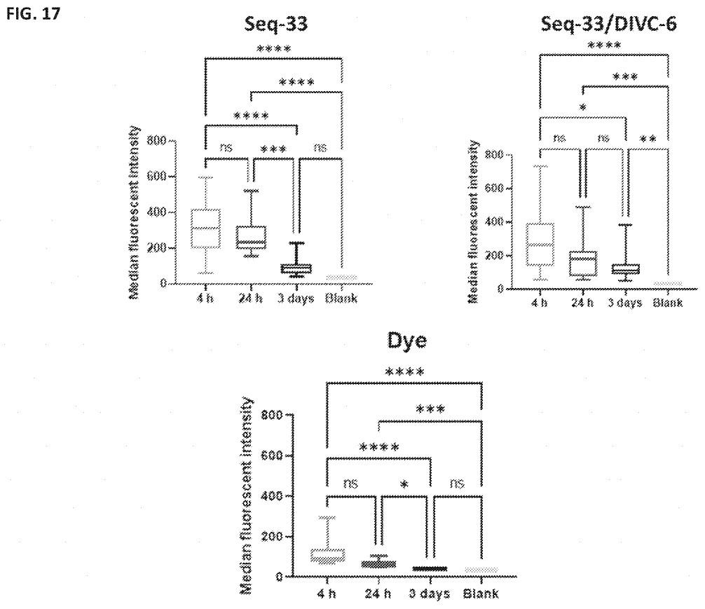

Further analysis of the lung distribution of the different compounds was performed by confocal microscopy. Lungs were collected at 4 hr, 24 hr and 72 hr post administration, fixed in formaldehyde 4% in Glucose 5%. Tissues were stained with Mito tracker red and nucleus with Hoesch. Confocal microscopy analysis are reported in FIGS. 16A-16B.

At 4 hr, both peptides and peptide/siRNA complexes mainly accumulated at the cell surface. Fluorescent signals are also reported in macrophages and in parenchyma, but none of the compound was detected in nuclei.

At 24 hr, peptides and peptide/siRNA complexes gave similar bright signal, with accumulation in macrophages and in cells types 1 and 2. In contrast, no signal was detected for free Cy 5.5 dye, which confirms the lung delivery specificity of the different compounds.

At 72 hr, both peptides and peptide/siRNA complexes are mainly located in the cytoplasm of type1/2 epithelial cells and macrophages.

These results suggested that Seq-17 and Seq-33 peptides are able to target lung epithelial cell type 1 and 2 and to significantly penetrate lung tissue when administrated intratracheally. As reported in FIG. 17, all compounds remain detectable in the lung for 72 hr, whereas the free dye is barely detectable after 24 hr.

The fact that both peptides and peptides/siRNA accumulated at the cell surface after intratracheal instillation, demonstrated that they constitute good candidate to prevent SARS-COV-2 initial infection.

### Example 18. Lung and Liver Toxicity Studies of Peptide and Peptide/siRNA Complex Inhibitors of SARS-CoV2

Lung and liver toxicity studies of Seq17 (LNCOV-15), seq 33 (LNCOV-18), seq17/DIVC-6 and Seq33/DIVC-6 complexes were performed in healthy 4 weeks old male C57BL/6J mice. Single dose of peptides or peptide/siRNA complexes were administrated via intratracheal instillation (200 μg) (Table 20). Saline solution was used as negative control. Samples were collected 12, 24, 48 and 72 hr post administration.

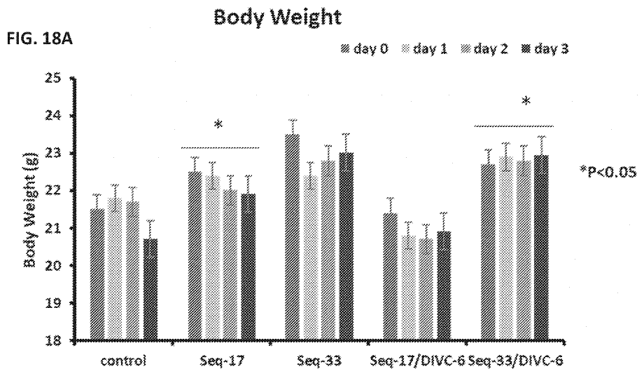

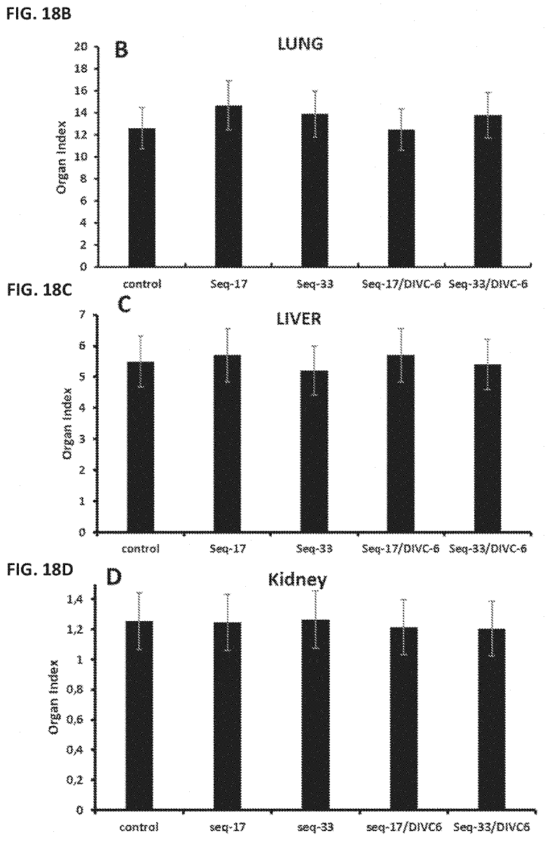

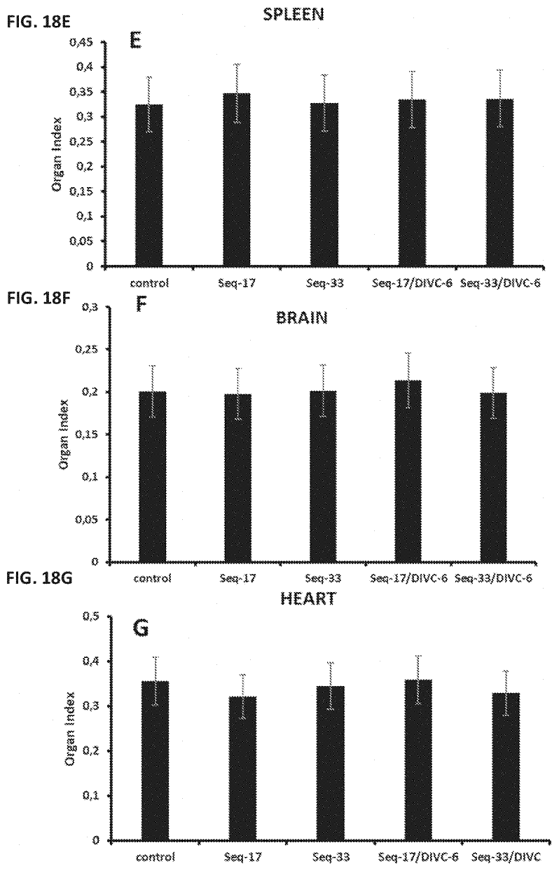

The body weight of the mice were recorded before intratracheal instillation (day 0), at day1, day 2 and before being sacrificed (day 3). The lung, liver, heart, kidney and brain were collected at day 3 and organ index were determined. As reported in FIGS. 18A-18G, statistical data analysis revealed no significant body weight change and no modification of organ index between the different groups.

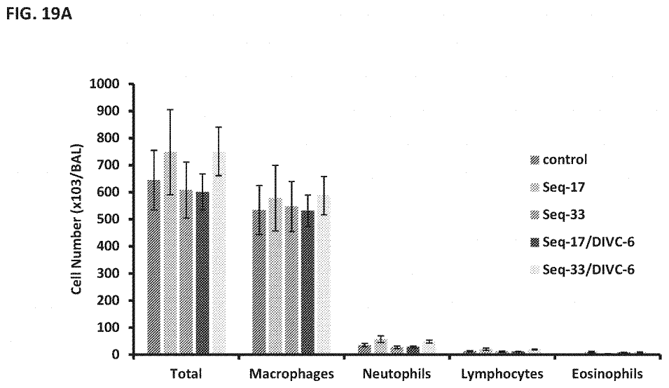

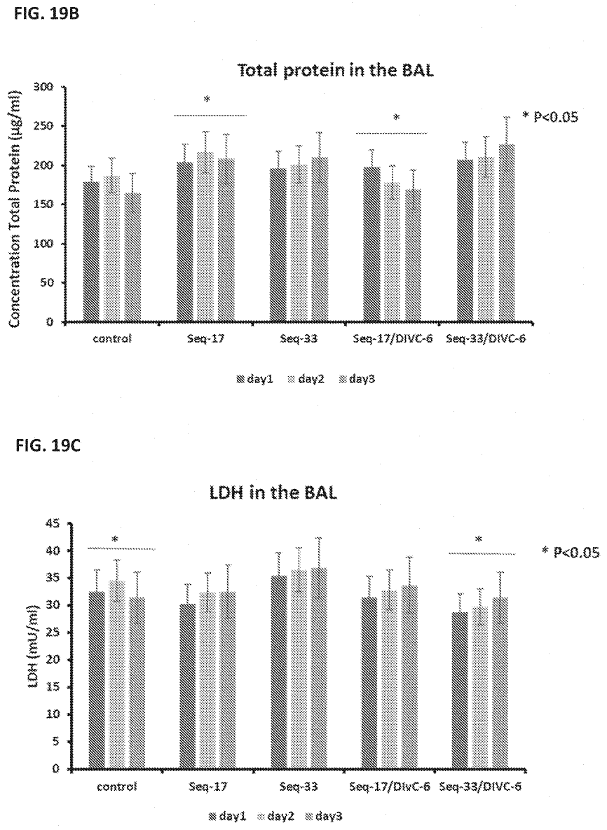

Bronchoalveolar lavage (BAL) were performed 2 days after instillation. The percentage of the cells, the total protein and level of LDH in the BAL were analyzed. Results were compared to saline buffer used as negative control. As reported in FIG. 19A, no obvious difference were observed between compound candidates and negative control. Only a slight increases of macrophages was detected in Seq-17 and Seq-33/DIVC-6 treated mice. Based on the statistical data analysis, there is no significant difference in LDH and total protein assay among the 5 groups (FIGS. 19B-19C).

The data demonstrated that neither peptide inhibitors nor peptide/siRNA complexes treatments induced pulmonary inflammations and that both treatments are well tolerated.

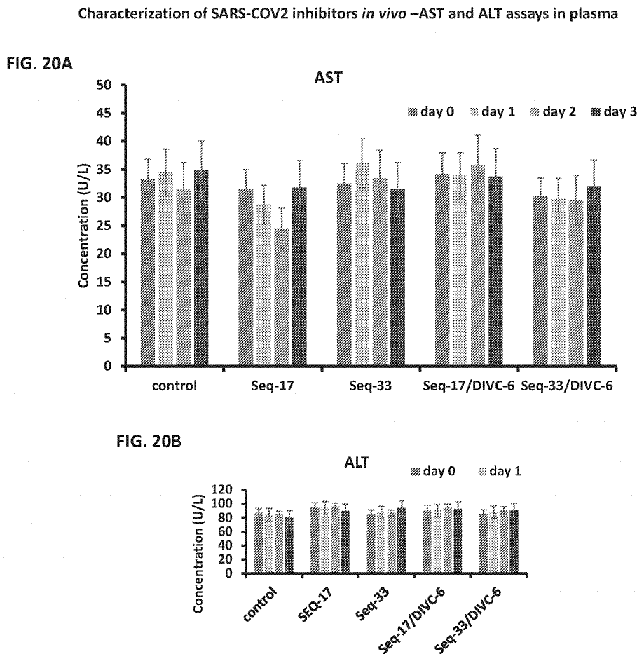

AST and ALT levels in the plasma were assayed 2 days after instillation. Based on the statistical data analysis, there is no significant difference in ALT or AST assay among the 5 groups (FIGS. 20A-20B). The data demonstrated that neither peptide inhibitors nor peptide/siRNA complexes upon lung instillation induced liver toxicity.

The lead SARS-COV-2 inhibitors; Seq-17, Seq-33 and Seq-17/DIVC-6 and Seq-33/DIVC6 are well tolerated upon intratracheal administration and no lung and liver toxicity have been associated to treatment.

SEQUENCE TABLE

| SEQ | | |
|---|---|---|
| ID NO. | Description | Nucleotide or Amino Acid Sequence |
| 1. | LNCOV-01 | SKVGGNYNYLYR |
| 2. | LNCOV-0 | KVGGNYNYL |
| 3. | LNCOV-1 | GNYNYLYRL |
| 4. | LNCOV-2 | GGNYNYLYRLFRK |
| 5. | LNCOV-3 | STEYQAGST |
| 6. | LNCOV-4 | CNGVEGFNC |
| 7. | LNCOV-5 | VEGENCYFP |
| 8. | LNCOV-6 | YFPLQSYGFQPTNGVGYQ |
| 9. | LNCOV-7 | SYGFQPTNGV |
| 10. | LNCOV-8 | GFQPTNGVK |
| 11. | LNCOV-9 | TNGVGYQPY |
| 12. | LNCOV-10 | Ac-S*KVGGNYNYLY*R-Ava/Ahx-LWRALWRLWRSLWRLLWK-NH2 |
| | | *Cyclization site |
| 13. | LNCOV-11 | Ac-K*VGGNYNYK*-Ava-LWRALWRLWRSLWRLLWK-NH2 |
| | | *Cyclization site |
| 14. | LNCOV-12 | Ac-G*NYNYLYRL*-Ava-LWRALWRLWRSLWRLLWK-NH2 |
| | | *Cyclization site |
| 15. | LNCOV-22 | Ac-G*NYNYLYRLFK*-Ava-LWRALWRLWRSLWRLLWK-NH2 |
| | | *Cyclization site |
| 16. | LNCOV-17 | Ac-T*EYQAGSTK*Ava-LWRALWRLWRSLWRLLWK-NH2 |
| | | *Cyclization site |
| 17. | LNCOV-15 | Ac-N*GVEGFNK*Ava-LWRALWRLWRSLWRLLWK-NH2 |
| | | *Cyclization site |
| 18. | LNCOV-16 | Ac-V*EGENCYF*Ava-LWRALWRLWRSLWRLLWK-NH2 |
| | | *Cyclization site |
| 19. | LNCOV-23 | Ac-YFPLQSYGFQPTNGVGYQ Ava- |
| | | LWRALWRLWRSLWRLLWK-NH2 |
| 20. | LNCOV-24 | Ac-S*YGFQPTNGV*-Ava-LWRALWRLWRSLWRLLWK-NH2 |
| | | *Cyclization site |
| 21. | LNCOV-13 | Ac-G*FQPTNGVK*-Ava-LWRALWRLWRSLWRLLWK-NH2 |
| | | *Cyclization site |
| 22. | LNCOV-14 | Ac-T*NGVGYQPYK*-Ava-LWRALWRLWRSLWRLLWK-NH2 |
| | | *Cyclization site |
| 23. | SBP-1 | Ac-IEEQAKTFLDKFNHEAEDLFYQS |
| 24. | LNCOV-19 | Ac-EQAKTFLDKFNHEAEDLF |
| 25. | LNCOV-33 | Ac-KTFLDKFNHEAEDLF |
| 26. | LNCOV-34 | Ac-KTFLDKFNHEAEDLFYS |
| 27. | LNCOV-27 | Ac-EQAKTFLDKFNHEAEDLF-P-KWSAFLKEQSTLAQMYG |
| 28. | LNCOV-20 | Ac-KTFLDKFNHEAEDLFPG-P-KWSAFLKEQSTLAQMYG |
| 29. | LNCOV-35 | Ac-TAWDLGKGDFRI |
| 30. | LNCOV-36 | Ac-NMTQGFWENSMLTD |
| 31. | LNCOV-37 | Ac-NMTQGFWEN-PGGP-TAWDLGK |
| 32. | LNCOV-21 | Ac-IEEQAKTFLDKFNHEAEDLFYQS-Ahx-LWRALWRLWRSLWRLLWK-NH2 |
| 33. | LNCOV-18 | Ac-EQAKTFLDKFNHEAEDLF-Ahx-LWRALWRLWRSLWRLLWK-NH2 |
| 34. | LNCOV-25 | Ac-KTFLDKFNHEAEDLF-Ahx-LWRALWRLWRSLWRLLWK-NH2 |
| 35. | LNCOV-26 | Ac-KTFLDKFNHEAEDLFYS-Ahx-LWRALWRLWRSLWRLLWK-NH2 |
| 36. | LNCOV-28 | Ac-TAWDLGKGDFRI-Ahx-LWRALWRLWRSLWRLLWK-NH2 |
| 37. | LNCOV-29 | Ac-NMTQGFWENSMLTD-Ahx-LWRALWRLWRSLWRLLWK-NH2 |
| 38. | LNCOV-30 | Ac-NMTQGFWENPGGPTAWDLGK-Ahx-LWRALWRLWRSLWRLLWK-NH2 |
| 39. | LNCOV-31 | Ac-EQAKTFLDKFNHEAEDLF-P-KWSAFLKEQSTLAQMYG-Ahx- |
| | | LWRALWRLWRSLWRLLWK-NH2 |
| 40. | LNCOV-32 | Ac-KTFLDKFNHEAEDLF-P-KWSAFLKEQSTLAQMYG- |
| | | Ahx-LWRALWRLWRSLWRLLWK-NH2 |
| 41. | LNCOV-33 | Ac-EQAKTFLDKFNHEAEDLF-ahx-RSAGWRWRLWRVRSWSR-NH2 |
| 42. | SPIKE | KVGGNYNY |
| 43. | SPIKE | GNYNYLYRLF |
| 44. | SPIKE | TEYQAGST |
| 45. | SPIKE | NGVEGEN |
| 46. | SPIKE | VEGENCYF |
| 47. | ACE2 | KTFLDKFNHEAEDLFY |
| 48. | ACE2 | KTFLDKFNHEAEDLFYS |
| 49. | ACE2 | KWSAFLKEQSTLAQMY |
| 50. | ACE2 | KWSAFLKEQSTLAQMYG |
| 51. | ACE2 | NMTQGFWEN |
| 52. | ACE2 | TAWDLGK |
| 53. | ADGN-100 | RWRLWRX₁X₂X₃X₄SR |
| | core motif | X₁ is V or S, X₂ is R, V, or A, X₃ is S or L, and X₄ is W or Y |
| 54. | ADGN-100 | RSX₁X₂X₃RWRLWRX₄X₅X₆X₇SR |
| | | X₁ is A or V, X₂ is G or L, X₃ is W or Y, X₄ is V or S, X₅ is R, |
| | | V, or A, X₆ is S or L, and X₇ is W or Y. |
| 55. | ADGN-100a | RSAGWRWRLWRVRSWSR |
| 56. | ADGN-100b | RSALYRWRLWRVRSWSR |
| 57. | ADGN-100c | RSALYRWRLWRSRSWSR |
| 58. | ADGN-100d | RSALYRWRLWRSALYSR |
| 59. | ADGN-100 | X₁KWRSX₂X₃X₄RWRLWRX₅X₆X₇X₈SR |
| | | X₁ is any amino acid or none, and X₂-X₈ are any amino acid |
| 60. | ADGN-100 | X₁KWRSX₂X₃X₄RWRLWRX₅X₆X₇X₈SR |
| | | X₁ is βA, S, or none, X₂ is A or V, X₃ is G or L, X₄ is W or Y, |
| | | X₅ is V or S, X₆ is R, V, or A, X₇ is S or L, and X₈ is W or Y. |
| 61. | ADGN-100a | KWRSAGWRWRLWRVRSWSR |
| 62. | ADGN-100b | KWRSALYRWRLWRVRSWSR |
| 63. | ADGN-100c | KWRSALYRWRLWRSRSWSR |
| 64. | ADGN-100d | KWRSALYRWRLWRSALYSR |
| 65. | ADGN-102 | RWRLWRWSR |
| | core motif | |
| 66. | ADGN-100-RI | RSWSRVRWLRWRWGASR |
| 67. | ADGN-100-RI | RSWSRVRWLRWRWGASRWK |
| 68. | ADGN-100 aa | RS_{S}AGWR_{S}WRLWRVRSWSR |
| | | the residues marked with a subscript “S” are linked by a |
| | | hydrocarbon linkage |
| 69. | ADGN-100 ab | R_{S}SAGWRWR_{S}LWRVRSWSR |
| | | the residues marked with a subscript “S” are linked by a |
| | | hydrocarbon linkage |
| 70. | ADGN-100 ac | RSAGWR_{S}WRLWRVR_{S}SWSR |
| | | the residues marked with a subscript “S” are linked by a |
| | | hydrocarbon linkage |
| 71. | ADGN-100 ba | RS_{S}ALYR_{S}WRLWRSRSWSR |
| | | the residues marked with a subscript “S” are linked by a |
| | | hydrocarbon linkage |
| 72. | ADGN-100 bb | R_{S}SALYRWR_{S}LWRSRSWSR |
| | | the residues marked with a subscript “S” are linked by a |
| | | hydrocarbon linkage |
| 73. | ADGN-100 bc | RSALYR_{S}WRLWRSR_{S}SWSR |
| | | the residues marked with a subscript “S” are linked by a |
| | | hydrocarbon linkage |
| 74. | ADGN-100 bd | RSALYRWR_{S}LWRS_{S}RSWSR |
| | | the residues marked with a subscript “S” are linked by a |
| | | hydrocarbon linkage |
| 75. | ADGN-100 be | RSALYRWRLWRS_{S}RSWS_{S}R |
| | | the residues marked with a subscript “S” are linked by a |
| | | hydrocarbon linkage |
| 76. | ADGN-100 ca | R_{S}SALYRWR_{S}LWRSALYSR |
| | | the residues marked with a subscript “S” are linked by a |
| | | hydrocarbon linkage |
| 77. | ADGN-100 cb | RS_{S}ALYR_{S}WRLWRSALYSR |
| | | the residues marked with a subscript “S” are linked by a |
| | | hydrocarbon linkage |
| 78. | ADGN-100 cc | RSALYRWR_{S}LWRS_{S}ALYSR |
| | | the residues marked with a subscript “S” are linked by a |
| | | hydrocarbon linkage |
| 79. | ADGN-100 cd | RSALYRWRLWRS_{S}ALYS_{S}R |
| | | the residues marked with a subscript “S” are linked by a |
| | | hydrocarbon linkage |
| 80. | VEPEP-6 1 | LX₁RALWX₈LX₂X₈X₃LWX₈LX₄X₅X₆X₇ |
| | | X₁ is F or W, X₂ is L, W, C or I, X₃ is S, A, N or T, X₄ is L or |
| | | W, X₅ is W or R, X₆ is K or R, X₇ is A or none, and X₈ is R or S |
| 81. | VEPEP-6 2 | LX₁LARWX₈LX₂X₈X₃LWX₈LX₄X₅X₆X7 |
| | | X₁ is F or W, X₂ is L, W, C or I, X₃ is S, A, N or T, X₄ is L or |
| | | W, X₅ is W or R, X₆ is K or R, X₇ is A or none, and X₈ is R or S |
| 82. | VEPEP-6 3 | LX₁ARLWX₈LX₂X₈X₃LWX₈LX₄X₅X₆X7 |
| | | X₁ is F or W, X₂ is L, W, C or I, X₃ is S, A, N or T, X₄ is L or |
| | | W, X₅ is W or R, X₆ is K or R, X₇ is A or none, and X₈ is R or S |
| 83. | VEPEP-6 4 | LX₁RALWRLX₂RX₃LWRLX₄X₅X₆X₇ |
| | | X₁ is F or W, X₂ is L, W, C or I, X₃ is S, A, N or T, X₄ is L or |
| | | W, X₅ is W or R, X₆ is K or R, and X₇ is A or none |
| 84. | VEPEP-6 5 | LX₁RALWRLX₂RX₃LWRLX₄X₅KX₆ |
| | | X₁ is F or W, X₂ is L or W, X₃ is S, A or N, X₄ is L or W, X₅ is |
| | | W or R, X₆ is A or none |
| 85. | VEPEP-6 6 | LFRALWRLLRX₁LWRLLWX₂ |
| | | X₁ is S or T, and X₂ is K or R |
| 86. | VEPEP-6 7 | LWRALWRLWRX₁LWRLLWX₂A |
| | | X₁ is S or T, and X₂ is K or R |
| 87. | VEPEP-6 8 | LWRALWRLX₃RX₁LWRLWRX₂A |
| | | X₁ is S or T, X₂ is K or R, and X₃ is L, C or I |
| 88. | VEPEP-6 9 | LWRALWRLWRX₁LWRLWRX₂A |
| | | X₁ is S or T, and X₂ is K or R |
| 89. | VEPEP-6 10 | LWRALWRLX₁RALWRLLWX₂A |
| | | X₁ is L or I. and X₂ is K or R |
| 90. | VEPEP-6 11 | LWRALWRLX₁RNLWRLLWX₂A |
| | | X₁ is L, C or I, and X₂ is K or R |
| 91. | VEPEP-6a | LFRALWRLLRSLWRLLWK |
| 92. | VEPEP-6b | LWRALWRLWRSLWRLLWKA |
| 93. | VEPEP-6c | LWRALWRLLRSLWRLWRKA |
| 94. | VEPEP-6d | LWRALWRLWRSLWRLWRKA |
| 95. | VEPEP-6e | LWRALWRLLRALWRLLWKA |
| 96. | VEPEP-6f | LWRALWRLLRNLWRLLWKA |
| 97. | ADGN-106 | LWRALWRLWRSLWRLLWK |
| 98. | ADGN-106-RI | KWLLRWLSRWLRWLARWL |
| 99. | ST-VEPEP-6a | LFRALWRₛLLRSₛLWRLLWK |
| | | the residues followed by an inferior “s” are linked by a |
| | | hydrocarbon linkage |
| 100. | ST-VEPEP-6aa | LFLARWRₛLLRSₛLWRLLWK |
| | | the residues followed by an inferior “s” are linked by a |
| | | hydrocarbon linkage |
| 101. | ST-VEPEP-6ab | LFRALWSₛLLRSₛLWRLLWK |
| | | the residues followed by an inferior “s” are linked by a |
| | | hydrocarbon linkage |
| 102. | ST-VEPEP-6ad | LFLARWSₛLLRSₛLWRLLWK |
| | | the residues followed by an inferior “s” are linked by a |
| | | hydrocarbon linkage |
| 103. | ST-VEPEP-6b | LFRALWRLLRₛSLWSₛLLWK |
| | | the residues followed by an inferior “s” are linked by a |
| | | hydrocarbon linkage |
| 104. | ST-VEPEP-6ba | LFLARWRLLRₛSLWSₛLLWK |
| | | the residues followed by an inferior “s” are linked by a |
| | | hydrocarbon linkage |
| 105. | ST-VEPEP-6bb | LFRALWRLLSₛSLWSₛLLWK |
| | | the residues followed by an inferior “s” are linked by a |
| | | hydrocarbon linkage |
| 106. | ST-VEPEP-6bd | LFLARWRLLSₛSLWSₛLLWK |
| | | the residues followed by an inferior “s” are linked by a |
| | | hydrocarbon linkage |
| 107. | ST-VEPEP-6c | LFARₛLWRLLRSₛLWRLLWK |
| | | the residues followed by an inferior “s” are linked by a |
| | | hydrocarbon linkage |
| 108. | VEPEP-9 1 | X₁X₂X₃WWX₄X₅WAX₆X₃X₇X₈X₉X₁₀X₁₁X₁₂WX₁₃R |
| | | X₁ is beta-A, S or none, X₂ is L or none, X₃ is R or none, X₄ is |
| | | L, R or G, X₅ is R, W or S, X₆ is S, P or T, X₇ is W or P, X₈ is |
| | | F, A or R, X₉ is S, L, P or R, X₁₀ is R or S, X₁₁ is W or none, |
| | | X₁₂ is A, R or none and X₁₃ is W or F, and wherein if X₃ is |
| | | none, then X₂, X₁₁ and X₁₂ are none as well |
| 109. | VEPEP-9 2 | X₁X₂RWWLRWAXGRWX₈X₉X₁₀WX₁₂WX₁₃R |
| | | X₁ is beta-A, S or none, X₂ is L or none, X₆ is S or P, X₈ is F or |
| | | A, X₉ is S, L or P, X₁₀ is R or S, X₁₂ is A or R, and X₁₃ is W or F |
| 110. | VEPEP9a1 | X₁LRWWLRWASRWFSRWAWWR |
| | | X₁ is beta-A, S or none |
| 111. | VEPEP9a2 | X₁LRWWLRWASRWASRWAWFR |
| | | X₁ is beta-A, S or none |
| 112. | VEPEP9b1 | X₁RWWLRWASRWALSWRWWR |
| | | X₁ is beta-A, S or none |
| 113. | VEPEP9b2 | X₁RWWLRWASRWFLSWRWWR |
| | | X₁ is beta-A, S or none |
| 114. | VEPEP9c1 | X₁RWWLRWAPRWFPSWRWWR |
| | | X₁ is beta-A, S or none |
| 115. | VEPEP9c2 | X₁RWWLRWASRWAPSWRWWR |
| | | X₁ is beta-A, S or none |
| 116. | VEPEP-9 3 | X₁WWX₄X₅WAX₆X₇X₈RX₁₀WWR |
| | | X₁ is beta-A, S or none, X₄ is R or G, X₅ is W or S, X₆ is S, T |
| | | or P, X₇ is W or P, X₈ is A or R, and X₁₀ is S or R |
| 117. | VEPEP9d | X₁WWRWWASWARSWWR |
| | | X₁ is beta-A, S or none |
| 118. | VEPEP9e | X₁WWGSWATPRRRWWR |
| | | X₁ is beta-A, S or none |
| 119. | VEPEP9f | X₁WWRWWAPWARSWWR |
| | | X₁ is beta-A, S or none |
| 120. | VEPEP-9 | ALRWWLRWASRWFSRWAWWR |
| 121. | VEPEP-9C | RWWLRWASRWFSRWAWR |
| 122. | VEPEP-3 | X₁X₂X₃X₄X₅X₂X₃X₄X₆X₇X₃X₈X₉X₁₀X₁₁X₁₂X₁₃ |
| | | X₁ is beta-A, S or none, X₂ is K, R or L (independently from |
| | | each other), X₃ is F or W (independently from each other), X₄ |
| | | is F, W or Y (independently from each other), X₅ is E, R or S, |
| | | X₆ is R, T or S, X₇ is E, R, or S, X₈ is none, F or W, X₉ is P or |
| | | R, X₁₀ is R or L, X₁₁ is K, W or R, X₁₂ is R or F, and X₁₃ is R |
| | | or K |
| 123. | VEPEP-3 1 | X₁X₂WX₄EX₂WX₄X₆X₇X₃PRX₁₁RX₁₃ |
| | | X₁ is beta-A, S or none, X₂ is K, R or L, X₃ is F or W, X₄ is F, |
| | | W or Y, X₅ is E, R or S, X₆ is R, T or S, X₇ is E, R, or S, X₈ is |
| | | none, F or W, X₉ is P or R, X₁₀ is R or L, X₁₁ is K, W or R, X₁₂ |
| | | is R or F, and X₁₃ is R or K |
| 124. | VEPEP-3 1a | X₁KWFERWFREWPRKRR |
| | | X₁ is beta-A, S or none |
| 125. | VEPEP-3 1b | X₁KWWERWWREWPRKRR |
| | | X₁ is beta-A, S or none |
| 126. | VEPEP-3 1c | X₁KWWERWWREWPRKRK |
| | | X₁ is beta-A, S or none |
| 127. | VEPEP-3 1d | X₁RWWEKWWTRWPRKRK |
| | | X₁ is beta-A, S or none |
| 128. | VEPEP-3 1e | X₁RWYEKWYTEFPRRRR |
| | | X₁ is beta-A, S or none |
| 129. | VEPEP-3 1S | X₁KX₁₄WWERWWRX₁₄WPRKRK |
| | | X₁ is beta-A, S or none, and X₁₄ is a non-natural amino acid, |
| | | and wherein there is a hydrocarbon linkage between the two |
| | | non-natural amino acids |
| 130. | VEPEP-3 2 | X₁X₂X₃WX₅X₁₀X₃WX₆X₇WX₈X₉X₁₀WX₁₂R |
| | | X₁ is beta-A, S or none, X₂ is K, R or L, X₃ is F or W, X₅ is R |
| | | or S, X₆ is R or S, X₇ is R or S, X₈ is F or W, X₉ is R or P, X₁₀ |
| | | is L or R, and X₁₂ is R or F |
| 131. | VEPEP-3 2a | X₁RWWRLWWRSWFRLWRR |
| | | X₁ is beta-A, S or none |
| 132. | VEPEP-3 2b | X₁LWWRRWWSRWWPRWRR |
| | | X₁ is beta-A, S or none |
| 133. | VEPEP-3 2c | X₁LWWSRWWRSWFRLWFR |
| | | X₁ is beta-A, S or none |
| 134. | VEPEP-3 2d | X₁KFWSRFWRSWFRLWRR |
| | | X₁ is beta-A, S or none |
| 135. | VEPEP-3 2S | X₁RWWX₁₄LWWRSWX₁₄RLWRR |
| | | X₁ is a beta-alanine, a serine or none, and X₁₄ is a non-natural |
| | | amino acid, and wherein there is a hydrocarbon linkage |
| | | between the two non-natural amino acids |
| 136. | VEPEP-3a | AKWFERWFREWPRKRR |
| 137. | VEPEP-3b | AKWWERWWREWPRKRR |
| 138. | VEPEP-4 | XWXRLXXXXXX |
| | | X in position 1 is beta-A, S or none; X in positions 3, 9 and 10 |
| | | are, independently from each other, W or F; X in position 6 is |
| | | R if X in position 8 is S, and X in position 6 is S if X in |
| | | position 8 is R; X in position 7 is L or none; X in position 11 is |
| | | R or none, and X in position 7 is L if X in position 11 is none |
| 139. | VEPEP-4 | X₁WWRLSLRWW |
| | | X₁ is beta-A, S or none |
| 140. | VEPEP-4 | X₁WFRLSLRFWR |
| | | X₁ is beta-A, S or none |
| 141. | VEPEP-4 | X₁WWRLRSWFR |
| | | X₁ is beta-A, S or none |
| 142. | VEPEP-4 | X₁WFRLSLRFW |
| | | X₁ is beta-A, S or none |
| 143. | VEPEP-5 | RXWXRLWXRLR |
| | | X in position 2 is R or S; and X in positions 4 and 8 are, |
| | | independently from each other, W or F |
| 144. | VEPEP-5 | X₁WWRLWWRLR |
| | | X₁ is beta-A, S or none |
| 145. | VEPEP-5 | X₁WFRLWFRLR |
| | | X₁ is beta-A, S or none |
| 146. | VEPEP-5 | X₁WFRLWWRLR |
| | | X₁ is beta-A, S or none |
| 147. | VEPEP-5 | X₁WWRLWFRLR |
| | | X₁ is beta-A, S or none |
| 148. | VEPEP-5 | X₁RWWRLWWRL |
| | | X₁ is beta-A, S or none |
| 149. | VEPEP-5 | X₁RSWFRLWFR |
| | | X₁ is beta-A, S or none |
| 150. | CADY | GLWRALWRLLRSLWRLLWKV |
| 151. | LNCOV-10 | S*KVGGNYNYLY*R |
| | cyclic peptide | *Cyclization site |
| 152. | LNCOV-11 | K*VGGNYNYK* |
| | cyclic peptide | *Cyclization site |
| 153. | LNCOV-12 | G*NYNYLYRL* |
| | cyclic peptide | *Cyclization site |
| 154. | LNCOV-22 | G*NYNYLYRLFK* |
| | cyclic peptide | *Cyclization site |
| 155. | LNCOV-17 | T EYQAGSTK |
| | cyclic peptide | *Cyclization site |
| 156. | LNCOV-15 | N*GVEGFNK |
| | cyclic peptide | *Cyclization site |
| 157. | LNCOV-16 | V*EGENCYF* |
| | cyclic peptide | *Cyclization site |
| 158. | LNCOV-24 | S*YGFQPTNGV* |
| | Cyclic peptide | *Cyclization site |
| 159. | LNCOV-13 | G*FQPTNGVK* |
| | Cyclic peptide | *Cyclization site |
| 160. | LNCOV-14 | T*NGVGYQPYK* |
| | Cyclic peptide | *Cyclization site |
| 161. | DIVC-1 | GCAACTGAGGGAGCCTTGAAT |
| 162. | DIVC-2 | GACCAGGAACTAATCAGACAA |
| 163. | DIVC-3 | GACAAGGCGTTCCAATTAACA |
| 164. | DIVC-4 | GACTTCCCTATGGTGCTAACA |
| 165. | DIVC-5 | GGCTGATGAAACTCAAGCCTT |
| 166. | DIVC-6 | GGCCAAACTGTCACTAAGAAA |
| 167. | DIVC-7 | GCAGACGTGGTCCAGAACAAA |
| 168. | DIVC-8 | GCCTCTTCTCGTTCCTCATCA |
| 169. | DIVC-9 | AAGCATATTGACGCATACAAA |
| 170. | DIVC-10 | GCAACAATCCATGAGCAGTGC |
| 171. | DIVC-31 | GGTGAAATCAAGGATGCTACT |
| 172. | DIVC-32 | GTGTAACTTCTTCAATTGTCA |
| 173. | DIVC-33 | CAAGTCCTATTTCTGAACATG |
| 174. | DIVC-34 | GAACATGTTACCTTCTTCATC |
| 175. | DIVC-35 | TGCAGAGTATAAACTTTGTAA |
| 176. | DIVC-81 | GTTGATGACCCGTGTCCTATT |
| 177. | DIVC-82 | GACCCGTGTCCTATTCACTTC |
| 178. | DIVC-83 | GTGGATGAGGCTGGTTCTAAA |
| 179. | DIVC-84 | GCGTTGTTCGTTCTATGAAGA |
| 180. | DIVC-85 | GTCCTATTCACTTCTATTCTA |
| 181. | PEP-1 | KETWWETWWTEWSQPKKKRKV |
| 182. | PEP-2 | KETWFETWFTEWSQPKKKRKV |
| 183. | PEP-3 | KWFETWFTEWPKKRK |
| 184. | MPG | GALFLGFLGAAGSTMGAWSQPKKKRKV |
| 185. | Targeting | YIGSR |
| | peptide | |
| 186. | Linker | GGGGS |
| 187. | Linker | SGGGG |
| 188. | LNCOV-33 | Ac-IEEQAKTFLDKFNHEAEDLFYQS-Ahx-RSAGWRWRLWRVRSWSR-NH2 |

1. A chimeric peptide comprising a blocking peptide connected to a stabilizing peptide, wherein the blocking peptide specifically blocks interaction between SPIKE and ACE2, and wherein the stabilizing peptide stabilizes secondary or tertiary structure of the blocking peptide.

2. The chimeric peptide of claim 1, wherein the blocking peptide comprises a loop sequence within the receptor-binding domain (RBD) of SPIKE.

3. The chimeric peptide of claim 2, wherein the loop sequence has a length of no more than about 20 amino acids.

4. The chimeric peptide of claim 3, wherein the loop sequence has a length of about 7 amino acids to about 18 amino acids.

5. The chimeric peptide of any one of claims 1-4, wherein the blocking peptide comprises a lysine (K) at the C-terminus.

6. The chimeric peptide of any one of claims 1-5, wherein the loop sequence is selected from the group consisting of SEQ ID NOs: 1-11 and 42-46.

7. The chimeric peptide of claim 6, wherein the loop sequence is selected from the group consisting of SEQ ID NOs: 1, 6, 8-11, 42 and 45.

8. The chimeric peptide of claim 1, wherein the blocking peptide comprises a sequence derived from a sequence within the extracellular domain of ACE2.

9. The chimeric peptide of claim 8, wherein the blocking peptide comprises a sequence selected from the group consisting of SEQ ID NOs: 23-31 and 47-52.

10. The chimeric peptide of claim 9, wherein the blocking peptide comprises a sequence selected from the group consisting of SEQ ID NOs: 23, 24, 26-28, 31 and 47-52.

11. The chimeric peptide of claim 2-10, wherein the loop sequence is cyclic.

12. The chimeric peptide of any one of claims 1-11, wherein the stabilizing peptide is connected to the C-terminus of the blocking peptide.

13. The chimeric peptide of any one of claims 1-11, wherein the stabilizing peptide is connected to the N-terminus of the blocking peptide.

14. The chimeric peptide of claim 12 or claim 13, wherein the stabilizing peptide has a length of about 12 amino acids to about 30 amino acids.

15. The chimeric peptide of any one of claims 8-14, wherein the blocking peptide and the stabilizing peptide each comprises a sequence derived from ACE2.

16. The chimeric peptide of claim 15, wherein the stabilizing peptide comprises a sequence set forth in SEQ ID NO: 49 or 50.

17. The chimeric peptide of any one of claims 1-14, wherein the stabilizing peptide comprises an amphipathic helix structure.

18. The chimeric peptide of claim 17, wherein the stabilizing peptide comprises an ADGN-100 peptide or a VEPEP-6 peptide.

19. The chimeric peptide of claim 18, wherein the stabilizing peptide comprises a sequence set forth in any one of SEQ ID NOs: 53-107.

20. The chimeric peptide of claim 19, wherein the stabilizing peptide comprises a sequence set forth in SEQ ID NO: 55 or 97.

21. The chimeric peptide of any one of claims 1-20, wherein the blocking peptide and the stabilizing peptide are connected via a linker.

22. The chimeric peptide of claim 21, wherein the linker is selected from the group consisting of a proline, a polyglycine linker moiety, a PEG moiety, Aun, Ava, and Ahx.

23. The chimeric peptide of claim 19, wherein the PEG moiety consists of about two to about seven ethylene glycol units.

24. The chimeric peptide of any one of claims 1-23, wherein the chimeric peptide comprises the amino acid sequence of any one of SEQ ID NOs: 12-22, 27, 28, and 31-41.

25. The chimeric peptide of claim 24, wherein the chimeric peptide comprises the amino acid sequence selected from the group consisting of SEQ ID NOs: 12, 17, 19-22, 27, 28, 31-33, 35, and 38-40.

26. A non-naturally occurring peptide comprising the amino acid sequence of any of SEQ ID NOs: 12-22, 24-41, and 151-160.

27. The non-naturally occurring peptide of claim 26, wherein the peptide comprises the amino acid sequence of any of SEQ ID NOs: 12, 17, 19-22, 24, 26-28, 31-33, 35, 38-40, 151, 156, and 158-160.

28. The non-naturally occurring peptide of claim 26 or 27, wherein the peptide has a length of no more than about 100 amino acids.

29. A siRNA comprising a nucleic acid sequence selected from the group consisting of SEQ ID NOs: 161-180.

30. The siRNA of claim 29, wherein the siRNA comprises a nucleic acid sequence set forth in SEQ ID NO: 163, 170, 166 or 168.

31. A complex comprising a) a cargo comprising the chimeric peptide of any of the claims 1-25, the peptide of claim 26 or 27, or A siRNA of claim 28 or claim 29, and b) a second peptide, wherein the peptide or siRNA is complexed with the second peptide.

32. The complex of claim 31, wherein the second peptide is a cell penetrating peptide selected from the group consisting of CADY, PEP-1 peptides, PEP-2 peptides, PEP-3 peptides, LNCOV peptides, VEPEP-3 peptides, VEPEP-6 peptides, VEPEP-9 peptides, and ADGN-100 peptides.

33. The complex of claim 31 or claim 32, wherein the molar ratio of the second peptide to the peptide or siRNA is between about 1:1 and about 80:1.

34. The complex of claim 33, wherein the molar ratio of the second peptide to the peptide is between about 2:1 to about 10:1.

35. The complex of claim 34, wherein the molar ratio of the second peptide to the siRNA is between about 5:1 to about 50:1.

36. The complex of any one of claims 31-35, wherein the complex comprises a) the chimeric peptide of any of the claims 1-25, or the peptide of any of the claims 26-28, and/or b) a siRNA of claim 29 or claim 30.

37. The complex of claim 36, wherein the siRNA comprises a nucleic acid sequence set forth in SEQ ID NO: 166.

38. The complex of claim 37, wherein the complex comprises a chimeric peptide comprising the amino acid sequence set forth in SEQ ID NO: 17 or 33.

39. A nanoparticle comprising the complex of any one of claims 30-38.

40. The nanoparticle of claim 39, wherein the nanoparticle has a diameter of no more than about 100 nm.

41. The nanoparticle of claim 40, wherein the nanoparticle has a diameter of about 40 to about 60 nm.

42. A pharmaceutical composition comprising a) the chimeric peptide of any of the claims 1-25, the peptide of any of the claims 26-28, the siRNA of claim 29 or claim 30, the complex or the nanoparticle of any of claims 31-41, and b) a pharmaceutically acceptable carrier.

43. The pharmaceutical composition of claim 42, wherein the composition comprises two or more complexes or nanoparticles, wherein the two or more complexes or nanoparticles comprise different cargos.

44. A method of preparing the complex or the nanoparticle of any one of claims 31-41, comprising combining the cargo with the second peptide.

45. A method of treating a SARS-CoV-2 infection in an individual, comprising administering to the individual an effective amount of the pharmaceutical composition of claim 42 or claim 43.

46. The method of claim 45, wherein the pharmaceutical composition is administered via nebulization or local lung or nasal delivery.

47. The method of claim 45 or claim 46, wherein the individual is a human.


SEQUENCE LISTING (188 sequences)

SEQ ID NO: 1 - protein, 12 aa, Artificial Sequence
  Synthetic Construct
SKVGGNYNYLYR

SEQ ID NO: 2 - protein, 9 aa, Artificial Sequence
  Synthetic Construct
KVGGNYNYL

SEQ ID NO: 3 - protein, 9 aa, Artificial Sequence
  Synthetic Construct
GNYNYLYRL

SEQ ID NO: 4 - protein, 13 aa, Artificial Sequence
  Synthetic Construct
GGNYNYLYRLFRK

SEQ ID NO: 5 - protein, 9 aa, Artificial Sequence
  Synthetic Construct
STEYQAGST

SEQ ID NO: 6 - protein, 9 aa, Artificial Sequence
  Synthetic Construct
CNGVEGFNC

SEQ ID NO: 7 - protein, 9 aa, Artificial Sequence
  Synthetic Construct
VEGFNCYFP

SEQ ID NO: 8 - protein, 18 aa, Artificial Sequence
  Synthetic Construct
YFPLQSYGFQPTNGVGYQ

SEQ ID NO: 9 - protein, 10 aa, Artificial Sequence
  Synthetic Construct
SYGFQPTNGV

SEQ ID NO: 10 - protein, 9 aa, Artificial Sequence
  Synthetic Construct
GFQPTNGVK

SEQ ID NO: 11 - protein, 9 aa, Artificial Sequence
  Synthetic Construct
TNGVGYQPY

SEQ ID NO: 12 - protein, 30 aa, Artificial Sequence
  Synthetic Construct
  VARIANT 1, 11, 12, 30: Modifications as shown in the Sequence Table of the specification
SKVGGNYNYLYRLWRALWRLWRSLWRLLWK

SEQ ID NO: 13 - protein, 27 aa, Artificial Sequence
  Synthetic Construct
  VARIANT 1, 9, 27: Modifications as shown in the Sequence Table of the specification
KVGGNYNYKLWRALWRLWRSLWRLLWK

SEQ ID NO: 14 - protein, 27 aa, Artificial Sequence
  Synthetic Construct
  VARIANT 1, 9, 27: Modifications as shown in the Sequence Table of the specification
GNYNYLYRLLWRALWRLWRSLWRLLWK

SEQ ID NO: 15 - protein, 29 aa, Artificial Sequence
  Synthetic Construct
  VARIANT 1, 11, 29: Modifications as shown in the Sequence Table of the specification
GNYNYLYRLFKLWRALWRLWRSLWRLLWK

SEQ ID NO: 16 - protein, 27 aa, Artificial Sequence
  Synthetic Construct
  VARIANT 1, 9, 27: Modifications as shown in the Sequence Table of the specification
TEYQAGSTKLWRALWRLWRSLWRLLWK

SEQ ID NO: 17 - protein, 26 aa, Artificial Sequence
  Synthetic Construct
  VARIANT 1, 8, 26: Modifications as shown in the Sequence Table of the specification
NGVEGFNKLWRALWRLWRSLWRLLWK

SEQ ID NO: 18 - protein, 26 aa, Artificial Sequence
  Synthetic Construct
  VARIANT 1, 8, 26: Modifications as shown in the Sequence Table of the specification
VEGFNCYFLWRALWRLWRSLWRLLWK

SEQ ID NO: 19 - protein, 36 aa, Artificial Sequence
  Synthetic Construct
  VARIANT 1, 18, 36: Modifications as shown in the Sequence Table of the specification
YFPLQSYGFQPTNGVGYQLWRALWRLWRSLWRLLWK

SEQ ID NO: 20 - protein, 28 aa, Artificial Sequence
  Synthetic Construct
  VARIANT 1, 10, 28: Modifications as shown in the Sequence Table of the specification
SYGFQPTNGVLWRALWRLWRSLWRLLWK

SEQ ID NO: 21 - protein, 27 aa, Artificial Sequence
  Synthetic Construct
  VARIANT 1, 9, 27: Modifications as shown in the Sequence Table of the specification
GFQPTNGVKLWRALWRLWRSLWRLLWK

SEQ ID NO: 22 - protein, 28 aa, Artificial Sequence
  Synthetic Construct
  VARIANT 1, 10, 28: Modifications as shown in the Sequence Table of the specification
TNGVGYQPYKLWRALWRLWRSLWRLLWK

SEQ ID NO: 23 - protein, 23 aa, Artificial Sequence
  Synthetic Construct
  VARIANT 1: Modifications as shown in the Sequence Table of the specification
IEEQAKTFLDKFNHEAEDLFYQS

SEQ ID NO: 24 - protein, 18 aa, Artificial Sequence
  Synthetic Construct
  VARIANT 1: Modifications as shown in the Sequence Table of the specification
EQAKTFLDKFNHEAEDLF

SEQ ID NO: 25 - protein, 15 aa, Artificial Sequence
  Synthetic Construct
  VARIANT 1: Modifications as shown in the Sequence Table of the specification
KTFLDKFNHEAEDLF

SEQ ID NO: 26 - protein, 17 aa, Artificial Sequence
  Synthetic Construct
  VARIANT 1: Modifications as shown in the Sequence Table of the specification
KTFLDKFNHEAEDLFYS

SEQ ID NO: 27 - protein, 35 aa, Artificial Sequence
  Synthetic Construct
  VARIANT 1: Modifications as shown in the Sequence Table of the specification
EQAKTFLDKFNHEAEDLFKWSAFLKEQSTLAQMYG

SEQ ID NO: 28 - protein, 34 aa, Artificial Sequence
  Synthetic Construct
  VARIANT 1: Modifications as shown in the Sequence Table of the specification
KTFLDKFNHEAEDLFPGKWSAFLKEQSTLAQMYG

SEQ ID NO: 29 - protein, 12 aa, Artificial Sequence
  Synthetic Construct
  VARIANT 1: Modifications as shown in the Sequence Table of the specification
TAWDLGKGDFRI

SEQ ID NO: 30 - protein, 14 aa, Artificial Sequence
  Synthetic Construct
  VARIANT 1: Modifications as shown in the Sequence Table of the specification
NMTQGFWENSMLTD

SEQ ID NO: 31 - protein, 20 aa, Artificial Sequence
  Synthetic Construct
  VARIANT 1: Modifications as shown in the Sequence Table of the specification
NMTQGFWENPGGPTAWDLGK

SEQ ID NO: 32 - protein, 41 aa, Artificial Sequence
  Synthetic Construct
  VARIANT 1, 23, 41: Modifications as shown in the Sequence Table of the specification
IEEQAKTFLDKFNHEAEDLFYQSLWRALWRLWRSLWRLLWK

SEQ ID NO: 33 - protein, 36 aa, Artificial Sequence
  Synthetic Construct
  VARIANT 1, 18, 36: Modifications as shown in the Sequence Table of the specification
EQAKTFLDKFNHEAEDLFLWRALWRLWRSLWRLLWK

SEQ ID NO: 34 - protein, 33 aa, Artificial Sequence
  Synthetic Construct
  VARIANT 1, 15, 33: Modifications as shown in the Sequence Table of the specification
KTFLDKFNHEAEDLFLWRALWRLWRSLWRLLWK

SEQ ID NO: 35 - protein, 35 aa, Artificial Sequence
  Synthetic Construct
  VARIANT 1, 17, 35: Modifications as shown in the Sequence Table of the specification
KTFLDKFNHEAEDLFYSLWRALWRLWRSLWRLLWK

SEQ ID NO: 36 - protein, 30 aa, Artificial Sequence
  Synthetic Construct
  VARIANT 1, 12, 30: Modifications as shown in the Sequence Table of the specification
TAWDLGKGDFRILWRALWRLWRSLWRLLWK

SEQ ID NO: 37 - protein, 32 aa, Artificial Sequence
  Synthetic Construct
  VARIANT 1, 14, 32: Modifications as shown in the Sequence Table of the specification
NMTQGFWENSMLTDLWRALWRLWRSLWRLLWK

SEQ ID NO: 38 - protein, 38 aa, Artificial Sequence
  Synthetic Construct
  VARIANT 1, 20, 38: Modifications as shown in the Sequence Table of the specification
NMTQGFWENPGGPTAWDLGKLWRALWRLWRSLWRLLWK

SEQ ID NO: 39 - protein, 53 aa, Artificial Sequence
  Synthetic Construct
  VARIANT 1, 35, 53: Modifications as shown in the Sequence Table of the specification
EQAKTFLDKFNHEAEDLFKWSAFLKEQSTLAQMYGLWRALWRLWRSLWRLLWK

SEQ ID NO: 40 - protein, 50 aa, Artificial Sequence
  Synthetic Construct
  VARIANT 1, 32, 50: Modifications as shown in the Sequence Table of the specification
KTFLDKFNHEAEDLFKWSAFLKEQSTLAQMYGLWRALWRLWRSLWRLLWK

SEQ ID NO: 41 - protein, 35 aa, Artificial Sequence
  Synthetic Construct
  VARIANT 1, 18, 35: Modifications as shown in the Sequence Table of the specification
EQAKTFLDKFNHEAEDLFRSAGWRWRLWRVRSWSR

SEQ ID NO: 42 - protein, 8 aa, Artificial Sequence
  Synthetic Construct
KVGGNYNY

SEQ ID NO: 43 - protein, 10 aa, Artificial Sequence
  Synthetic Construct
GNYNYLYRLF

SEQ ID NO: 44 - protein, 8 aa, Artificial Sequence
  Synthetic Construct
TEYQAGST

SEQ ID NO: 45 - protein, 7 aa, Artificial Sequence
  Synthetic Construct
NGVEGFN

SEQ ID NO: 46 - protein, 8 aa, Artificial Sequence
  Synthetic Construct
VEGFNCYF

SEQ ID NO: 47 - protein, 16 aa, Artificial Sequence
  Synthetic Construct
KTFLDKFNHEAEDLFY

SEQ ID NO: 48 - protein, 17 aa, Artificial Sequence
  Synthetic Construct
KTFLDKFNHEAEDLFYS

SEQ ID NO: 49 - protein, 16 aa, Artificial Sequence
  Synthetic Construct
KWSAFLKEQSTLAQMY

SEQ ID NO: 50 - protein, 17 aa, Artificial Sequence
  Synthetic Construct
KWSAFLKEQSTLAQMYG

SEQ ID NO: 51 - protein, 9 aa, Artificial Sequence
  Synthetic Construct
NMTQGFWEN

SEQ ID NO: 52 - protein, 7 aa, Artificial Sequence
  Synthetic Construct
TAWDLGK

SEQ ID NO: 53 - protein, 12 aa, Artificial Sequence
  Synthetic Construct
  VARIANT 7: Xaa = V or S
  VARIANT 8: Xaa = R, V, or A
  VARIANT 9: Xaa = S or L
  VARIANT 10: Xaa = W or Y
RWRLWRXXXXSR

SEQ ID NO: 54 - protein, 17 aa, Artificial Sequence
  Synthetic Construct
  VARIANT 3: Xaa = A or V
  VARIANT 4: Xaa = G or L
  VARIANT 5: Xaa = W or Y
  VARIANT 12: Xaa = V or S
  VARIANT 13: Xaa = R, V, or A
  VARIANT 14: Xaa = S or L
  VARIANT 15: Xaa = W or Y
RSXXXRWRLWRXXXXSR

SEQ ID NO: 55 - protein, 17 aa, Artificial Sequence
  Synthetic Construct
RSAGWRWRLWRVRSWSR

SEQ ID NO: 56 - protein, 17 aa, Artificial Sequence
  Synthetic Construct
RSALYRWRLWRVRSWSR

SEQ ID NO: 57 - protein, 17 aa, Artificial Sequence
  Synthetic Construct
RSALYRWRLWRSRSWSR

SEQ ID NO: 58 - protein, 17 aa, Artificial Sequence
  Synthetic Construct
RSALYRWRLWRSALYSR

SEQ ID NO: 59 - protein, 20 aa, Artificial Sequence
  Synthetic Construct
  VARIANT 1: Xaa = Any Amino Acid or none
  VARIANT 6, 7, 8, 15, 16, 17, 18: Xaa = Any Amino Acid
XKWRSXXXRWRLWRXXXXSR

SEQ ID NO: 60 - protein, 20 aa, Artificial Sequence
  Synthetic Construct
  VARIANT 1: Xaa = beta-A, S, or none
  VARIANT 6: Xaa = A or V
  VARIANT 7: Xaa = G or L
  VARIANT 8: Xaa = W or Y
  VARIANT 15: Xaa = V or S
  VARIANT 16: Xaa = R, V, or A
  VARIANT 17: Xaa = S or L
  VARIANT 18: Xaa = W or Y
XKWRSXXXRWRLWRXXXXSR

SEQ ID NO: 61 - protein, 19 aa, Artificial Sequence
  Synthetic Construct
KWRSAGWRWRLWRVRSWSR

SEQ ID NO: 62 - protein, 19 aa, Artificial Sequence
  Synthetic Construct
KWRSALYRWRLWRVRSWSR

SEQ ID NO: 63 - protein, 19 aa, Artificial Sequence
  Synthetic Construct
KWRSALYRWRLWRSRSWSR

SEQ ID NO: 64 - protein, 19 aa, Artificial Sequence
  Synthetic Construct
KWRSALYRWRLWRSALYSR

SEQ ID NO: 65 - protein, 9 aa, Artificial Sequence
  Synthetic Construct
RWRLWRWSR

SEQ ID NO: 66 - protein, 17 aa, Artificial Sequence
  Synthetic Construct
RSWSRVRWLRWRWGASR

SEQ ID NO: 67 - protein, 19 aa, Artificial Sequence
  Synthetic Construct
RSWSRVRWLRWRWGASRWK

SEQ ID NO: 68 - protein, 17 aa, Artificial Sequence
  Synthetic Construct
  VARIANT 2, 6: Modifications as shown in the Sequence Table of the specification
RSAGWRWRLWRVRSWSR

SEQ ID NO: 69 - protein, 17 aa, Artificial Sequence
  Synthetic Construct
  VARIANT 1, 8: Modifications as shown in the Sequence Table of the specification
RSAGWRWRLWRVRSWSR

SEQ ID NO: 70 - protein, 17 aa, Artificial Sequence
  Synthetic Construct
  VARIANT 6, 13: Modifications as shown in the Sequence Table of the specification
RSAGWRWRLWRVRSWSR

SEQ ID NO: 71 - protein, 17 aa, Artificial Sequence
  Synthetic Construct
  VARIANT 2, 6: Modifications as shown in the Sequence Table of the specification
RSALYRWRLWRSRSWSR

SEQ ID NO: 72 - protein, 17 aa, Artificial Sequence
  Synthetic Construct
  VARIANT 1, 8: Modifications as shown in the Sequence Table of the specification
RSALYRWRLWRSRSWSR

SEQ ID NO: 73 - protein, 17 aa, Artificial Sequence
  Synthetic Construct
  VARIANT 6, 13: Modifications as shown in the Sequence Table of the specification
RSALYRWRLWRSRSWSR

SEQ ID NO: 74 - protein, 17 aa, Artificial Sequence
  Synthetic Construct
  VARIANT 8, 12: Modifications as shown in the Sequence Table of the specification
RSALYRWRLWRSRSWSR

SEQ ID NO: 75 - protein, 17 aa, Artificial Sequence
  Synthetic Construct
  VARIANT 12, 16: Modifications as shown in the Sequence Table of the specification
RSALYRWRLWRSRSWSR

SEQ ID NO: 76 - protein, 17 aa, Artificial Sequence
  Synthetic Construct
  VARIANT 1, 8: Modifications as shown in the Sequence Table of the specification
RSALYRWRLWRSALYSR

SEQ ID NO: 77 - protein, 17 aa, Artificial Sequence
  Synthetic Construct
  VARIANT 2, 6: Modifications as shown in the Sequence Table of the specification
RSALYRWRLWRSALYSR

SEQ ID NO: 78 - protein, 17 aa, Artificial Sequence
  Synthetic Construct
  VARIANT 8, 12: Modifications as shown in the Sequence Table of the specification
RSALYRWRLWRSALYSR

SEQ ID NO: 79 - protein, 17 aa, Artificial Sequence
  Synthetic Construct
  VARIANT 12, 16: Modifications as shown in the Sequence Table of the specification
RSALYRWRLWRSALYSR

SEQ ID NO: 80 - protein, 19 aa, Artificial Sequence
  Synthetic Construct
  VARIANT 2: Xaa = F or W
  VARIANT 7: Xaa = R or S
  VARIANT 9: Xaa = L, W, C or I
  VARIANT 10: Xaa = R or S
  VARIANT 11: Xaa = S, A, N or T
  VARIANT 14: Xaa = R or S
  VARIANT 16: Xaa = L or W
  VARIANT 17: Xaa = W or R
  VARIANT 18: Xaa = K or R
  VARIANT 19: Xaa = A or none
LXRALWXLXXXLWXLXXXX

SEQ ID NO: 81 - protein, 19 aa, Artificial Sequence
  Synthetic Construct
  VARIANT 2: Xaa = F or W
  VARIANT 7: Xaa = R or S
  VARIANT 9: Xaa = L, W, C or I
  VARIANT 10: Xaa = R or S
  VARIANT 11: Xaa = S, A, N or T
  VARIANT 14: Xaa = R or S
  VARIANT 16: Xaa = L or W
  VARIANT 17: Xaa = W or R
  VARIANT 18: Xaa = K or R
  VARIANT 19: Xaa = A or none
LXLARWXLXXXLWXLXXXX

SEQ ID NO: 82 - protein, 19 aa, Artificial Sequence
  Synthetic Construct
  VARIANT 2: Xaa = F or W
  VARIANT 7: Xaa = R or S
  VARIANT 9: Xaa = L, W, C or I
  VARIANT 10: Xaa = R or S
  VARIANT 11: Xaa = S, A, N or T
  VARIANT 14: Xaa = R or S
  VARIANT 16: Xaa = L or W
  VARIANT 17: Xaa = W or R
  VARIANT 18: Xaa = K or R
  VARIANT 19: Xaa = A or none
LXARLWXLXXXLWXLXXXX

SEQ ID NO: 83 - protein, 19 aa, Artificial Sequence
  Synthetic Construct
  VARIANT 2: Xaa = F or W
  VARIANT 9: Xaa = L, W, C or I
  VARIANT 11: Xaa = S, A, N or T
  VARIANT 16: Xaa = L or W
  VARIANT 17: Xaa = W or R
  VARIANT 18: Xaa = K or R
  VARIANT 19: Xaa = A or none
LXRALWRLXRXLWRLXXXX

SEQ ID NO: 84 - protein, 19 aa, Artificial Sequence
  Synthetic Construct
  VARIANT 2: Xaa = F or W
  VARIANT 9: Xaa = L or W
  VARIANT 11: Xaa = S, A or N
  VARIANT 16: Xaa = L or W
  VARIANT 17: Xaa = W or R
  VARIANT 19: Xaa = A or none
LXRALWRLXRXLWRLXXKX

SEQ ID NO: 85 - protein, 18 aa, Artificial Sequence
  Synthetic Construct
  VARIANT 11: Xaa = S or T
  VARIANT 18: Xaa = K or R
LFRALWRLLRXLWRLLWX

SEQ ID NO: 86 - protein, 19 aa, Artificial Sequence
  Synthetic Construct
  VARIANT 11: Xaa = S or T
  VARIANT 18: Xaa = K or R
LWRALWRLWRXLWRLLWXA

SEQ ID NO: 87 - protein, 19 aa, Artificial Sequence
  Synthetic Construct
  VARIANT 9: Xaa = L, C or I
  VARIANT 11: Xaa = S or T
  VARIANT 18: Xaa = K or R
LWRALWRLXRXLWRLWRXA

SEQ ID NO: 88 - protein, 19 aa, Artificial Sequence
  Synthetic Construct
  VARIANT 11: Xaa = S or T
  VARIANT 18: Xaa = K or R
LWRALWRLWRXLWRLWRXA

SEQ ID NO: 89 - protein, 19 aa, Artificial Sequence
  Synthetic Construct
  VARIANT 9: Xaa = L or I
  VARIANT 18: Xaa = K or R
LWRALWRLXRALWRLLWXA

SEQ ID NO: 90 - protein, 19 aa, Artificial Sequence
  Synthetic Construct
  VARIANT 9: Xaa = L, C or I
  VARIANT 18: Xaa = K or R
LWRALWRLXRNLWRLLWXA

SEQ ID NO: 91 - protein, 18 aa, Artificial Sequence
  Synthetic Construct
LFRALWRLLRSLWRLLWK

SEQ ID NO: 92 - protein, 19 aa, Artificial Sequence
  Synthetic Construct
LWRALWRLWRSLWRLLWKA

SEQ ID NO: 93 - protein, 19 aa, Artificial Sequence
  Synthetic Construct
LWRALWRLLRSLWRLWRKA

SEQ ID NO: 94 - protein, 19 aa, Artificial Sequence
  Synthetic Construct
LWRALWRLWRSLWRLWRKA

SEQ ID NO: 95 - protein, 19 aa, Artificial Sequence
  Synthetic Construct
LWRALWRLLRALWRLLWKA

SEQ ID NO: 96 - protein, 19 aa, Artificial Sequence
  Synthetic Construct
LWRALWRLLRNLWRLLWKA

SEQ ID NO: 97 - protein, 18 aa, Artificial Sequence
  Synthetic Construct
LWRALWRLWRSLWRLLWK

SEQ ID NO: 98 - protein, 18 aa, Artificial Sequence
  Synthetic Construct
KWLLRWLSRWLRWLARWL

SEQ ID NO: 99 - protein, 18 aa, Artificial Sequence
  Synthetic Construct
  VARIANT 7, 11: Modifications as shown in the Sequence Table of the specification
LFRALWRLLRSLWRLLWK

SEQ ID NO: 100 - protein, 18 aa, Artificial Sequence
  Synthetic Construct
  VARIANT 7, 11: Modifications as shown in the Sequence Table of the specification
LFLARWRLLRSLWRLLWK

SEQ ID NO: 101 - protein, 18 aa, Artificial Sequence
  Synthetic Construct
  VARIANT 7, 11: Modifications as shown in the Sequence Table of the specification
LFRALWSLLRSLWRLLWK

SEQ ID NO: 102 - protein, 18 aa, Artificial Sequence
  Synthetic Construct
  VARIANT 7, 11: Modifications as shown in the Sequence Table of the specification
LFLARWSLLRSLWRLLWK

SEQ ID NO: 103 - protein, 18 aa, Artificial Sequence
  Synthetic Construct
  VARIANT 10, 14: Modifications as shown in the Sequence Table of the specification
LFRALWRLLRSLWSLLWK

SEQ ID NO: 104 - protein, 18 aa, Artificial Sequence
  Synthetic Construct
  VARIANT 10, 14: Modifications as shown in the Sequence Table of the specification
LFLARWRLLRSLWSLLWK

SEQ ID NO: 105 - protein, 18 aa, Artificial Sequence
  Synthetic Construct
  VARIANT 10, 14: Modifications as shown in the Sequence Table of the specification
LFRALWRLLSSLWSLLWK

SEQ ID NO: 106 - protein, 18 aa, Artificial Sequence
  Synthetic Construct
  VARIANT 10, 14: Modifications as shown in the Sequence Table of the specification
LFLARWRLLSSLWSLLWK

SEQ ID NO: 107 - protein, 18 aa, Artificial Sequence
  Synthetic Construct
  VARIANT 4, 11: Modifications as shown in the Sequence Table of the specification
LFARLWRLLRSLWRLLWK

SEQ ID NO: 108 - protein, 20 aa, Artificial Sequence
  Synthetic Construct
  VARIANT 1: Xaa = beta-A, S or none
  VARIANT 2: Xaa = L or none
  VARIANT 3: Xaa = R or none
  VARIANT 6: Xaa = L, R, or G
  VARIANT 7: Xaa = R, W or S
  VARIANT 10: Xaa = S, P or T
  VARIANT 11: Xaa = R or none
  VARIANT 12: Xaa = W or P
  VARIANT 13: Xaa = F, A or R
  VARIANT 14: Xaa = S, L, P or R
  VARIANT 15: Xaa = R or S
  VARIANT 16: Xaa = W or none
  VARIANT 17: Xaa = A, R or none
  VARIANT 19: Xaa = W or F
XXXWWXXWAXXXXXXXXWXR

SEQ ID NO: 109 - protein, 20 aa, Artificial Sequence
  Synthetic Construct
  VARIANT 1: Xaa = beta-A, S or none
  VARIANT 2: Xaa = L or none
  VARIANT 10: Xaa = S or P
  VARIANT 13: Xaa = F or A
  VARIANT 14: Xaa = S, L or P
  VARIANT 15: Xaa = R or S
  VARIANT 17: Xaa = A or R
  VARIANT 19: Xaa = W or F
XXRWWLRWAXRWXXXWXWXR

SEQ ID NO: 110 - protein, 20 aa, Artificial Sequence
  Synthetic Construct
  VARIANT 1: Xaa = beta-A, S or none
XLRWWLRWASRWFSRWAWWR

SEQ ID NO: 111 - protein, 20 aa, Artificial Sequence
  Synthetic Construct
  VARIANT 1: Xaa = beta-A, S or none
XLRWWLRWASRWASRWAWFR

SEQ ID NO: 112 - protein, 19 aa, Artificial Sequence
  Synthetic Construct
  VARIANT 1: Xaa = beta-A, S or none
XRWWLRWASRWALSWRWWR

SEQ ID NO: 113 - protein, 19 aa, Artificial Sequence
  Synthetic Construct
  VARIANT 1: Xaa = beta-A, S or none
XRWWLRWASRWFLSWRWWR

SEQ ID NO: 114 - protein, 19 aa, Artificial Sequence
  Synthetic Construct
  VARIANT 1: Xaa = beta-A, S or none
XRWWLRWAPRWFPSWRWWR

SEQ ID NO: 115 - protein, 19 aa, Artificial Sequence
  Synthetic Construct
  VARIANT 1: Xaa = beta-A, S or none
XRWWLRWASRWAPSWRWWR

SEQ ID NO: 116 - protein, 15 aa, Artificial Sequence
  Synthetic Construct
  VARIANT 1: Xaa = beta-A, S or none
  VARIANT 4: Xaa = R or G
  VARIANT 5: Xaa = W or S
  VARIANT 8: Xaa = S, T or P
  VARIANT 9: Xaa = W or P
  VARIANT 10: Xaa = A or R
  VARIANT 12: Xaa = S or R
XWWXXWAXXXRXWWR

SEQ ID NO: 117 - protein, 15 aa, Artificial Sequence
  Synthetic Construct
  VARIANT 1: Xaa = beta-A, S or none
XWWRWWASWARSWWR

SEQ ID NO: 118 - protein, 15 aa, Artificial Sequence
  Synthetic Construct
  VARIANT 1: Xaa = beta-A, S or none
XWWGSWATPRRRWWR

SEQ ID NO: 119 - protein, 15 aa, Artificial Sequence
  Synthetic Construct
  VARIANT 1: Xaa = beta-A, S or none
XWWRWWAPWARSWWR

SEQ ID NO: 120 - protein, 20 aa, Artificial Sequence
  Synthetic Construct
ALRWWLRWASRWFSRWAWWR

SEQ ID NO: 121 - protein, 17 aa, Artificial Sequence
  Synthetic Construct
RWWLRWASRWFSRWAWR

SEQ ID NO: 122 - protein, 17 aa, Artificial Sequence
  Synthetic Construct
  VARIANT 1: Xaa = beta-A, S or none
  VARIANT 2: Xaa = K, R or L
  VARIANT 3: Xaa = F or W
  VARIANT 4: Xaa = F, W or Y
  VARIANT 5: Xaa = E, R or S
  VARIANT 6: Xaa = K, R or L
  VARIANT 7: Xaa = F or W
  VARIANT 8: Xaa = F, W or Y
  VARIANT 9: Xaa = R, T or S
  VARIANT 10: Xaa = E, R or S
  VARIANT 11: Xaa = F or W
  VARIANT 12: Xaa = none, F or W
  VARIANT 13: Xaa = P or R
  VARIANT 14: Xaa = R or L
  VARIANT 15: Xaa = K, W or R
  VARIANT 16: Xaa = R or F
  VARIANT 17: Xaa = R or K
XXXXXXXXXXXXXXXXX

SEQ ID NO: 123 - protein, 16 aa, Artificial Sequence
  Synthetic Construct
  VARIANT 1: Xaa = beta-A, S or none
  VARIANT 2: Xaa = K, R or L
  VARIANT 4: Xaa = F, W or Y
  VARIANT 6: Xaa = K, R or L
  VARIANT 8: Xaa = F, W or Y
  VARIANT 9: Xaa = R, T or S
  VARIANT 10: Xaa = E, R or S
  VARIANT 11: Xaa = F or W
  VARIANT 14: Xaa = K, W or R
  VARIANT 16: Xaa = R or K
XXWXEXWXXXXPRXRX

SEQ ID NO: 124 - protein, 16 aa, Artificial Sequence
  Synthetic Construct
  VARIANT 1: Xaa = beta-A, S or none
XKWFERWFREWPRKRR

SEQ ID NO: 125 - protein, 16 aa, Artificial Sequence
  Synthetic Construct
  VARIANT 1: Xaa = beta-A, S or none
XKWWERWWREWPRKRR

SEQ ID NO: 126 - protein, 16 aa, Artificial Sequence
  Synthetic Construct
  VARIANT 1: Xaa = beta-A, S or none
XKWWERWWREWPRKRK

SEQ ID NO: 127 - protein, 16 aa, Artificial Sequence
  Synthetic Construct
  VARIANT 1: Xaa = beta-A, S or none
XRWWEKWWTRWPRKRK

SEQ ID NO: 128 - protein, 16 aa, Artificial Sequence
  Synthetic Construct
  VARIANT 1: Xaa = beta-A, S or none
XRWYEKWYTEFPRRRR

SEQ ID NO: 129 - protein, 17 aa, Artificial Sequence
  Synthetic Construct
  VARIANT 1: Xaa = beta-A, S or none
  VARIANT 3, 11: Xaa =  non-natural amino acid, wherein there is a hydrocarbon linkage between the two non-natural amino acids
XKXWWERWWRXWPRKRK

SEQ ID NO: 130 - protein, 17 aa, Artificial Sequence
  Synthetic Construct
  VARIANT 1: Xaa = beta-A, S or none
  VARIANT 2: Xaa = K, R or L
  VARIANT 3: Xaa = F or W
  VARIANT 5: Xaa = R or S
  VARIANT 6: Xaa = L or R
  VARIANT 7: Xaa = F or W
  VARIANT 9: Xaa = R or S
  VARIANT 10: Xaa = R or S
  VARIANT 12: Xaa = F or W
  VARIANT 13: Xaa = R or P
  VARIANT 14: Xaa = L or R
  VARIANT 16: Xaa = R or F
XXXWXXXWXXWXXXWXR

SEQ ID NO: 131 - protein, 17 aa, Artificial Sequence
  Synthetic Construct
  VARIANT 1: Xaa = beta-A, S or none
XRWWRLWWRSWFRLWRR

SEQ ID NO: 132 - protein, 17 aa, Artificial Sequence
  Synthetic Construct
  VARIANT 1: Xaa = beta-A, S or none
XLWWRRWWSRWWPRWRR

SEQ ID NO: 133 - protein, 17 aa, Artificial Sequence
  Synthetic Construct
  VARIANT 1: Xaa = beta-A, S or none
XLWWSRWWRSWFRLWFR

SEQ ID NO: 134 - protein, 17 aa, Artificial Sequence
  Synthetic Construct
  VARIANT 1: Xaa = beta-A, S or none
XKFWSRFWRSWFRLWRR

SEQ ID NO: 135 - protein, 17 aa, Artificial Sequence
  Synthetic Construct
  VARIANT 1: Xaa = beta-A, S or none
  VARIANT 5, 12: Xaa = non-natural amino acid, and wherein there is a hydrocarbon linkage between the two non-natural amino acids
XRWWXLWWRSWXRLWRR

SEQ ID NO: 136 - protein, 16 aa, Artificial Sequence
  Synthetic Construct
AKWFERWFREWPRKRR

SEQ ID NO: 137 - protein, 16 aa, Artificial Sequence
  Synthetic Construct
AKWWERWWREWPRKRR

SEQ ID NO: 138 - protein, 11 aa, Artificial Sequence
  Synthetic Construct
  VARIANT 1: Xaa = beta-A, S or none
  VARIANT 3: Xaa = W or F
  VARIANT 6: Xaa = R when position 8 is S, or S when position 8 is R
  VARIANT 7: Xaa = L or none
  VARIANT 8: Xaa = S or R
  VARIANT 9: Xaa = W or F
  VARIANT 10: Xaa = W or F
  VARIANT 11: Xaa = R or none
XWXRLXXXXXX

SEQ ID NO: 139 - protein, 10 aa, Artificial Sequence
  Synthetic Construct
  VARIANT 1: Xaa = beta-A, S or none
XWWRLSLRWW

SEQ ID NO: 140 - protein, 11 aa, Artificial Sequence
  Synthetic Construct
  VARIANT 1: Xaa = beta-A, S or none
XWFRLSLRFWR

SEQ ID NO: 141 - protein, 10 aa, Artificial Sequence
  Synthetic Construct
  VARIANT 1: Xaa = beta-A, S or none
XWWRLRSWFR

SEQ ID NO: 142 - protein, 10 aa, Artificial Sequence
  Synthetic Construct
  VARIANT 1: Xaa = beta-A, S or none
XWFRLSLRFW

SEQ ID NO: 143 - protein, 11 aa, Artificial Sequence
  Synthetic Construct
  VARIANT 2: Xaa = R or S
  VARIANT 4, 8: Xaa = W or F
RXWXRLWXRLR

SEQ ID NO: 144 - protein, 10 aa, Artificial Sequence
  Synthetic Construct
  VARIANT 1: Xaa = beta-A, S or none
XWWRLWWRLR

SEQ ID NO: 145 - protein, 10 aa, Artificial Sequence
  Synthetic Construct
  VARIANT 1: Xaa = beta-A, S or none
XWFRLWFRLR

SEQ ID NO: 146 - protein, 10 aa, Artificial Sequence
  Synthetic Construct
  VARIANT 1: Xaa = beta-A, S or none
XWFRLWWRLR

SEQ ID NO: 147 - protein, 10 aa, Artificial Sequence
  Synthetic Construct
  VARIANT 1: Xaa = beta-A, S or none
XWWRLWFRLR

SEQ ID NO: 148 - protein, 10 aa, Artificial Sequence
  Synthetic Construct
  VARIANT 1: Xaa = beta-A, S or none
XRWWRLWWRL

SEQ ID NO: 149 - protein, 10 aa, Artificial Sequence
  Synthetic Construct
  VARIANT 1: Xaa = beta-A, S or none
XRSWFRLWFR

SEQ ID NO: 150 - protein, 20 aa, Artificial Sequence
  Synthetic Construct
GLWRALWRLLRSLWRLLWKV

SEQ ID NO: 151 - protein, 12 aa, Artificial Sequence
  Synthetic Construct
  VARIANT 1, 11: Modifications as shown in the Sequence Table of the specification
SKVGGNYNYLYR

SEQ ID NO: 152 - protein, 9 aa, Artificial Sequence
  Synthetic Construct
  VARIANT 1, 9: Modifications as shown in the Sequence Table ofthe specification
KVGGNYNYK

SEQ ID NO: 153 - protein, 9 aa, Artificial Sequence
  Synthetic Construct
  VARIANT 1, 9: Modifications as shown in the Sequence Table of the specification
GNYNYLYRL

SEQ ID NO: 154 - protein, 11 aa, Artificial Sequence
  Synthetic Construct
  VARIANT 1, 11: Modifications as shown in the Sequence Table of the specification
GNYNYLYRLFK

SEQ ID NO: 155 - protein, 9 aa, Artificial Sequence
  Synthetic Construct
  VARIANT 1, 9: Modifications as shown in the Sequence Table of the specification
TEYQAGSTK

SEQ ID NO: 156 - protein, 8 aa, Artificial Sequence
  Synthetic Construct
  VARIANT 1, 8: Modifications as shown in the Sequence Table of the specification
NGVEGFNK

SEQ ID NO: 157 - protein, 8 aa, Artificial Sequence
  Synthetic Construct
  VARIANT 1, 8: Modifications as shown in the Sequence Table of the specification
VEGFNCYF

SEQ ID NO: 158 - protein, 10 aa, Artificial Sequence
  Synthetic Construct
  VARIANT 1, 10: Modifications as shown in the Sequence Table of the specification
SYGFQPTNGV

SEQ ID NO: 159 - protein, 9 aa, Artificial Sequence
  Synthetic Construct
  VARIANT 1, 9: Modifications as shown in the Sequence Table of the specification
GFQPTNGVK

SEQ ID NO: 160 - protein, 10 aa, Artificial Sequence
  Synthetic Construct
  VARIANT 1, 10: Modifications as shown in the Sequence Table of the specification
TNGVGYQPYK

SEQ ID NO: 161 - DNA, 21 bp, Artificial Sequence
  Synthetic Construct
gcaactgagggagccttgaat

SEQ ID NO: 162 - DNA, 21 bp, Artificial Sequence
  Synthetic Construct
gaccaggaactaatcagacaa

SEQ ID NO: 163 - DNA, 21 bp, Artificial Sequence
  Synthetic Construct
gacaaggcgttccaattaaca

SEQ ID NO: 164 - DNA, 21 bp, Artificial Sequence
  Synthetic Construct
gacttccctatggtgctaaca

SEQ ID NO: 165 - DNA, 21 bp, Artificial Sequence
  Synthetic Construct
ggctgatgaaactcaagcctt

SEQ ID NO: 166 - DNA, 21 bp, Artificial Sequence
  Synthetic Construct
ggccaaactgtcactaagaaa

SEQ ID NO: 167 - DNA, 21 bp, Artificial Sequence
  Synthetic Construct
gcagacgtggtccagaacaaa

SEQ ID NO: 168 - DNA, 21 bp, Artificial Sequence
  Synthetic Construct
gcctcttctcgttcctcatca

SEQ ID NO: 169 - DNA, 21 bp, Artificial Sequence
  Synthetic Construct
aagcatattgacgcatacaaa

SEQ ID NO: 170 - DNA, 21 bp, Artificial Sequence
  Synthetic Construct
gcaacaatccatgagcagtgc

SEQ ID NO: 171 - DNA, 21 bp, Artificial Sequence
  Synthetic Construct
ggtgaaatcaaggatgctact

SEQ ID NO: 172 - DNA, 21 bp, Artificial Sequence
  Synthetic Construct
gtgtaacttcttcaattgtca

SEQ ID NO: 173 - DNA, 21 bp, Artificial Sequence
  Synthetic Construct
caagtcctatttctgaacatg

SEQ ID NO: 174 - DNA, 21 bp, Artificial Sequence
  Synthetic Construct
gaacatgttaccttcttcatc

SEQ ID NO: 175 - DNA, 21 bp, Artificial Sequence
  Synthetic Construct
tgcagagtataaactttgtaa

SEQ ID NO: 176 - DNA, 21 bp, Artificial Sequence
  Synthetic Construct
gttgatgacccgtgtcctatt

SEQ ID NO: 177 - DNA, 21 bp, Artificial Sequence
  Synthetic Construct
gacccgtgtcctattcacttc

SEQ ID NO: 178 - DNA, 21 bp, Artificial Sequence
  Synthetic Construct
gtggatgaggctggttctaaa

SEQ ID NO: 179 - DNA, 21 bp, Artificial Sequence
  Synthetic Construct
gcgttgttcgttctatgaaga

SEQ ID NO: 180 - DNA, 21 bp, Artificial Sequence
  Synthetic Construct
gtcctattcacttctattcta

SEQ ID NO: 181 - protein, 21 aa, Artificial Sequence
  Synthetic Construct
KETWWETWWTEWSQPKKKRKV

SEQ ID NO: 182 - protein, 21 aa, Artificial Sequence
  Synthetic Construct
KETWFETWFTEWSQPKKKRKV

SEQ ID NO: 183 - protein, 15 aa, Artificial Sequence
  Synthetic Construct
KWFETWFTEWPKKRK

SEQ ID NO: 184 - protein, 27 aa, Artificial Sequence
  Synthetic Construct
GALFLGFLGAAGSTMGAWSQPKKKRKV

SEQ ID NO: 185 - protein, 5 aa, Artificial Sequence
  Synthetic Construct
YIGSR

SEQ ID NO: 186 - protein, 5 aa, Artificial Sequence
  Synthetic Construct
GGGGS

SEQ ID NO: 187 - protein, 5 aa, Artificial Sequence
  Synthetic Construct
SGGGG

SEQ ID NO: 188 - protein, 40 aa, Artificial Sequence
  Synthetic Construct
  VARIANT 1, 23, 40: Modifications as shown in the Sequence Table of the specification
IEEQAKTFLDKFNHEAEDLFYQSRSAGWRWRLWRVRSWSR